(19) United States
(12) Patent Application Publication
(10) Pub. No.: US 2024/0336823 A1
(43) Pub. Date: Oct. 10, 2024

(54) REFRIGERANT COMPOSITIONS COMPRISING Z-1,3,3,3-TETRAFLUOROPROPENE, METHODS OF MAKING SAME, AND USES THEREOF

(71) Applicant: THE CHEMOURS COMPANY FC, LLC, Wilmington, DE (US)

(72) Inventors: SHENG PENG, Hockessin, DE (US); Luke David Simoni, Wilmington, DE (US); Joshua Hughes, Wilmington, DE (US)

(73) Assignee: THE CHEMOURS COMPANY FC, LLC, Wilmington, DE (US)

(21) Appl. No.: 18/627,510

(22) Filed: Apr. 5, 2024

Related U.S. Application Data
(60) Provisional application No. 63/457,627, filed on Apr. 6, 2023.
(60) Provisional application No. 63/466,460, filed on May 15, 2023.

(51) Int. Cl. C09K 5/04; C07C 17/25; C07C 21/18

(52) U.S. Cl. CPC C09K 5/045; C07C 17/25; C07C 21/18; C09K 2205/122; C09K 2205/126; C09K 2205/128; C09K 2205/22

(57) ABSTRACT

Disclosed herein are fluoroolefin refrigerant compositions, methods of producing the same, methods and systems using the same, and systems containing the Z-HFO-1234ze refrigerant compositions. The inventive compositions are useful as refrigerants in air conditioning, refrigeration systems, such as chillers, and heat pumps.

### CROSS REFERENCE TO RELATED APPLICATIONS

This application claims priority to U.S. Provisional Application No. 63/457,627, filed on Apr. 6, 2023, and U.S. Provisional Application No. 63/466,460, filed on May 15, 2023, the disclosure of each of which is herein incorporated by reference in its entirety.

### FIELD OF THE INVENTION

The present invention is directed to fluoroolefin refrigerant compositions, methods of producing the same, methods and systems using the same, and systems containing the Z-HFO-1234ze refrigerant compositions.

### BACKGROUND OF THE INVENTION

The fluorocarbon industry has been working for the past few decades to find replacement refrigerants for the ozone depleting chlorofluorocarbons (CFCs) and hydrochlorofluorocarbons (HCFCs) being phased out as a result of the Montreal Protocol. The solution for many applications has been the commercialization of hydrofluorocarbon (HFC) compounds for use as refrigerants, solvents, fire extinguishing agents, blowing agents and propellants. These new compounds, such as HFC refrigerants, HFC-134a and HFC-125 being the most widely used at this time, have zero ozone depletion potential (ODP) and thus are not affected by the current regulatory phase-out as a result of the Montreal Protocol. In addition to ozone depleting concerns, global warming is another environmental concern in many of these applications. HFC refrigerants such as HFC-134a and HFC-125 respectively have global warming potentials (GWP) of 1,300 and 3,170 according to the UN's IPCC Fifth Assessment Report (AR5).

This regulatory landscape is continuously evolving, taking into consideration properties beyond just ODP and GWP. More particularly, there is a need for refrigerant compositions that not only meet low ODP standards and have low global warming potentials, but that also exhibit low or no flammability, provide superior performance in a variety of applications and which meet the standards of evolving regulations.

There is a need in this art for new refrigerant compositions that meet evolving regulations as well as provide heat transfer and refrigerant characteristics that meet or exceed the effectiveness of conventional refrigerants and refrigerant blends.

HFO-1234ze(Z) (CF₃CH═CHF), like HFO-1234yf, has zero ozone depletion and very low global warming potential, and has thus been identified as a potential useful refrigerant. For example, U.S. Pat. No. 7,862,742 discloses compositions comprising HFO-1234ze and HFO-1234yf. U.S. Pat. No. 9,302,962 discloses methods for making HFO-1234ze. The disclosures of U.S. Pat. Nos. 7,862,742 and 9,302,962 are hereby incorporated by reference in their entireties. HFO-1234ze(Z) (CF₃CH═CHF), which has zero ozone depletion and very low global warming potential and with a boiling point of 9.7° C., HFO-1234ze(Z) possesses physical properties that make it an attractive option for heat pump and chiller applications, including flooded evaporator heat pumps and flooded chillers, as a single fluid or in blends.

The instant invention solves certain problems associated with conventional refrigerants and provides HFO-1234ze(Z)-based compositions which meet the evolving regulatory landscape. The present invention also provides for economical manufacturing processes that provide such compositions.

### SUMMARY OF THE INVENTION

Certain embodiments disclosed herein relate to processes for producing the Z isomer of a fluoropropene of formula CF₃CH═CHF in quantities greater than the E-isomer.

Certain embodiments disclosed herein relate to processes for producing the Z isomer of a fluoropropene of formula CF₃CH═CHF in quantities greater than the E-isomer, prior to isomerization of the E-isomer to the Z-isomer.

One embodiment of the present invention is directed to a fluoropropene composition comprising Z-1,3,3,3-tetrafluoropropene.

In one embodiment, the present invention relates to a composition comprising Z-1,3,3,3-tetrafluoropropene and at least one additional compound selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In one embodiment, the total amount of the additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In some embodiments, compositions of the present invention comprise HFO-1234ze(Z), HFC-236fa, HFC-245fa and one or more additional compounds selected from HFO-1234ze(E), HFC-263fb, HFO-1234zc, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFO-1327 isomer, HFO-1336mzz(E), CFO-1112a, HFC-227ea and HFC-245cb. In one embodiment, the total amount of the HFC-236fa, HFC-245fa and additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In some embodiments, compositions of the present invention comprise HFO-1234ze(Z), HFC-236fa, HFO-1234zc and one or more additional compounds selected from HFO-1234ze(E), HFC-263fb, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFO-1327 isomer, HFO-1336mzz(E), CFO-1112a, HFC-227ea and HFC-245cb. In one embodiment, the total amount of the HFC-236fa, HFO-1234zc and additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In some embodiments, compositions of the present invention comprise HFO-1234ze(Z), HFO-1234zc, HFC-245fa, HFO-1233zd(E) and one or more additional compounds selected from HFO-1234ze(E), HFC-263fb, HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb. In one embodiment, the total amount of the HFO-1234zc, HFC-245fa, HFO-1233zd(E) and additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In some embodiments, compositions of the present invention comprise HFO-1234ze(Z), HFO-1234zc, CFC-114 and one or more additional compounds selected from HFO-1234ze(E), HFC-263fb, HFC-245fa, HCFO-1233zd(E), HCFO-1233zd(Z), HCFO-1233xf, HCFC-124, HCC-40, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb. In one embodiment, the total amount of the HFO-1234zc, CFC-114 and additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In some embodiments, compositions of the present invention comprise HFO-1234ze(Z), HFO-1336mzz(E), HFO-1327 isomer and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, CFO-1112a, HFC-227ea and HFC-245cb. In one embodiment, the total amount of the HFO-1336mzz(E), HFO-1327 isomer and additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In some embodiments, compositions of the present invention comprise HFO-1234ze(Z), HFO-1336mzz(E), HFC-236fa and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFO-1327 isomer, CFO-1112a, HFC-227ea and HFC-245cb. In one embodiment, the total amount of the HFO-1336mzz(E), HFC-236fa and additional compounds is greater than 0 and less than 1 wt %, or less than 0.5 wt %, or less than 0.4 wt %, or less than 0.3 wt %, or less than 0.2 wt % or less than 0.1 wt % based on the total composition. In one embodiment, the total amount of the additional compounds is about 0.1 wt % based on the total composition.

In addition, the present disclosure includes methods of producing a fluoropropene of formula CF₃CH═CHF, particularly the Z isomer. The method comprises contacting a starting material in the gas phase or liquid phase with a catalyst, optionally in the presence of one of an oxygen containing gas or a fluorinating agent such as hydrogen fluoride, to form a reaction mixture comprising Z-1,3,3,3-tetrafluoropropene, E-1,3,3,3-tetrafluoropropene, and optionally additional constituents.

In one embodiment, the method further comprises separating the Z-1,3,3,3-tetrafluoropropene from the E-isomer and any additional constituents, if present, and recovering the Z-1,3,3,3-tetrafluoropropene.

In certain embodiments, the Z- and E-isomers are separated, the Z-isomer is recovered, and the E isomer is converted to the Z-isomer to increase the yield of the Z-isomer.

In another embodiment, the present disclosure describes a method of producing a fluoropropene of formula CF₃CH═CHF, particularly the Z isomer. The method comprises reacting HCC-240fa with hydrogen fluoride in the presence of a fluorinated catalyst, to form a reaction mixture comprising Z-1,3,3,3-tetrafluoropropene, E-1,3,3,3-tetrafluoropropene, and optionally additional constituents. In one embodiment, the hydrogen fluoride is preferably anhydrous or substantially anhydrous. In one embodiment, the method further comprises separating the Z-1,3,3,3-tetrafluoropropene from the E-isomer and any additional constituents, if present, and recovering the Z-1,3,3,3-tetrafluoropropene, and optionally converting the E-isomer to Z-1,3,3,3-tetrafluoropropene.

In another embodiment, the present disclosure includes a method of producing a fluoropropene of formula CF₃CH═CHF, particularly the Z isomer. The method comprises reacting HCFO-1233zd(E) and/or HCFO-1233zd(Z) with hydrogen fluoride in the presence of a fluorinated catalyst, to form a reaction mixture comprising Z-1,3,3,3-tetrafluoropropene, E-1,3,3,3-tetrafluoropropene, and optionally additional constituents. In one embodiment, the hydrogen fluoride is preferably anhydrous or substantially anhydrous. In one embodiment, the method further comprises separating the Z-1,3,3,3-tetrafluoropropene from the E-isomer and any additional constituents, if present, and recovering the Z-1,3,3,3-tetrafluoropropene.

One embodiment of the invention relates to any of the foregoing methods or combination thereof, wherein the fluorinated catalyst is a fluorinated chromium catalyst, including but not limited to Cr₂O₃, supported or unsupported on a carrier.

One embodiment of the invention relates to any of the foregoing methods or combination thereof, wherein the method further comprising recovering the E-1,3,3,3-tetrafluoropropene and converting the recovered E-1,3,3,3-tetrafluoropropene to the Z-isomer.

One embodiment of the invention relates to the foregoing E-isomer to Z-isomer converting steps, wherein the converting comprises isomerizing the E-isomer to the Z-isomer by contacting the E-1,3,3,3-tetrafluoropropene in the gas phase with at least one fluorinated catalyst, optionally in the presence of an oxygen containing gas, to form Z-1,3,3,3-tetrafluoropropene.

One embodiment of the invention relates to the foregoing E- to Z-isomer conversions, wherein 2% to 70% of the E-1,3,3,3-tetrafluoropropene is converted to the Z-isomer, and the method further comprises optionally recovering unconverted E-isomer and repeating the conversion by recycling unconverted E-isomer.

Further still, the present disclosure includes fluoropropene compositions formed from any of the foregoing methods or combinations thereof.

In another embodiment, the composition comprises a tetrafluoroolefin, and optionally one or more components selected from chlorofluoroolefins, hydrofluorocarbons, hydrocarbons, hydrofluoroolefins, as a heat transfer medium or blend.

In one embodiment, the composition comprises HFO-1234ze(Z) and at least one compound selected from HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224yd(E), CFO-1112(E), CFO-1112(Z), HFC-245fa, HFC-236fa, HFC-227ea, trans-1,2-dichloroethylene, HFO-1132(E), HFO-1132(Z), HFC-152a, HFC-134a, HFC-134, HFC-32, HFC-125, carbon dioxide, isobutene, propane, butane, isobutane, pentane and isopentane.

In one embodiment, the composition comprises HFO-1234ze(Z) and at least one compound selected from HFO-1336mzz(Z), HFO-1336mzz(E), HFC-245fa, isobutane, trans-1,2-dichloroethene, HCFO-1224 yd(Z), HCFO-1224yd(E), HFC-134 and HFC-227ea.

In one embodiment, the composition comprises HFO-1234ze(Z) and at least one compound selected from HFO-1336mzz(Z), HFO-1336mzz(E), HCFO-1233zd(E), HCFO-1233zd(Z), HFO-1234ze(E), HFC-236fa, HFC-134, HFC-152a, HFC-245fa, HFO-1132(E), and HFO-1132(Z).

In some embodiments, compositions of the present invention are free of or substantially free of Group A Fluorinated Substances, as defined herein.

In some embodiments, degradation products of compositions of the present invention are free of or substantially free of Group A Fluorinated Substances, as defined herein.

In some embodiments, compositions of the present invention comprise HFO-1234ze(Z) and HFO-1336mzz(Z).

In some embodiments, compositions of the present invention comprise HFO-1234ze(Z) and HFO-1336mzz(E).

In some embodiments, compositions of the present invention comprise HFO-1234ze(Z), HFO-1336mzz(E) and HFO-1336mzz(Z).

In some embodiments, compositions of the present invention comprise HFO-1234ze(Z), HFO-1336mzz(E) and HFC-245fa.

In some embodiments, compositions of the present invention comprise HFO-1234ze(Z), HFO-1336mzz(E) and isobutane.

In some embodiments, compositions of the present invention comprise HFO-1234ze(Z), HFO-1336mzz(E) and HFC-227ea.

In some embodiments, compositions of the present invention comprise HFO-1234ze(Z), HFO-1336mzz(E), HFO-1336mzz(Z) and HFC-134.

In some embodiments, compositions of the present invention comprise HFO-1234ze(Z), HFO-1336mzz(Z) and HCO-1130(E).

In some embodiments, compositions of the present invention comprise HFO-1234ze(Z), HFO-1336mzz(Z) and HCFO-1224 yd(Z).

In one embodiment, any of the blend compositions disclosed herein further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a and HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb. In some embodiments, compositions of the present invention comprise HFO-1234ze(Z), HFO-1336mzz(E) and HFO-1336mzz(Z). In some embodiments, compositions of the present invention comprise Z-1,3,3,3-tetrafluoropropene in an amount of 27 wt % to 72 wt %, HFO-1336mzz(E) in an amount of 0.5 wt % to 70.5 wt %, and %, HFO-1336mzz(Z) in an amount of 0.5 wt % to 27.5 wt %, based on the total composition.

In some embodiments, compositions of the present invention comprise HFO-1234ze(Z), HFO-1336mzz(E) and HFO-1336mzz(Z), and further comprise (i) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, (ii) one or more additional compounds selected from HFO-1336mzz(E), HFO-1327mz, HFO-1326mxz(Z), HFO-1326mxz(E), HFC-356mff, CHFC-346mdf, and HFC-263fb, and (iii) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

According to any of the foregoing embodiments, also disclosed herein are compositions that have a GWP of 300 or less, preferably 150 or less, more preferably 75 or less.

According to any of the foregoing embodiments, also disclosed herein are HFO-1234(Z) compositions and blends that have a flammability classification of 1, 2 L or 2 as determined by ASHRAE Standard 34 and ASTM E681-09.

According to any of the foregoing embodiments, also disclosed herein are compositions further comprising at least one lubricant.

According to any of the foregoing embodiments, also disclosed herein are compositions wherein the lubricant is selected from the group consisting of polyalkylene glycols, polyol esters, polyvinyl ethers, poly-alpha-olefins, and combinations thereof.

According to any of the foregoing embodiments, the amount of lubricant can range from about 1 wt % to about 20 wt %, about 1 wt % to about 7 wt %, and, in some cases, about 1 wt % to about 3 wt % of the total composition.

According to any of the foregoing embodiments, also disclosed herein are compositions further comprising at least one acid scavenger.

According to some embodiment, the one or more acid scavengers is/are selected from the group epoxides, amines and hindered amines.

According to any of the foregoing embodiments, also disclosed herein are compositions comprising HFO-1234ze(Z) and HFO-1234yf, and further comprising at least one olefin polymerization inhibitor.

According to any of the foregoing embodiments, also disclosed herein are compositions wherein the olefin polymerization inhibitor is selected from the group of d-limonene, l-limonene, β-pinene, a-pinene, a-terpinene, β-terpinene, y-terpinene, and o-terpinene, and mixtures of two or more thereof, and ethane, propane, cyclopropane, propylene, butane, butene, isobutane, isobutene, 2-methylbutane, meta-, ortho- or para-xylene, alpha, methyl styrene; alpha, 2-dimethyl styrene; alpha, 3-dimethyl styrene; or alpha, 4-dimethyl styrene.

According to any of the foregoing embodiments, also disclosed herein is a method for producing heating in a high temperature heat pump comprising condensing any of the foregoing compositions in a condenser, wherein the compositions allow operation at condenser temperatures higher than achievable with working fluids used in similar commonly used systems, because R-1234ze(Z) has a high critical temperature of 150.1° C., compared with, for example, critical temperatures of 94.7° C. for HFO-1234yf or 109.4° C. for E-HFO-1234ze.

According to the foregoing embodiment, the condenser operating temperature is greater than about 55° C., preferably from about 55° C. to about 150° C.

According to any of the foregoing embodiments, also disclosed herein a high temperature heat pump comprising a condenser and any of the foregoing compositions, wherein the condenser operating temperature is greater than about 55° C., greater than 65° C., greater than 75° C., greater than 85° C., greater than 95° C., greater than 100° C. to about 150° C., greater than about 150° C., greater than about 160° C., or greater than about 170° C.

According to any of the foregoing embodiments, also disclosed herein a method for raising the maximum feasible condenser operating temperature in a heat pump apparatus suitable relative to the maximum feasible condenser operating temperature when one of R-1233zdE, R-1336mzzE and R-1336mzzZ is used as the heat pump working fluid. This method comprises charging the heat pump with a working fluid comprising any of the foregoing compositions.

In some embodiments, the present invention is directed to a heat pump utilizing a working fluid comprising a HFO-1234ze(Z) composition according to any of the embodiments disclosed herein. In some embodiments, the heat pump is a high temperature heat pump. In some embodiments, the heat pump is a flooded evaporator heat pump system. In some embodiments, the heat pump is a cascade or auto-cascade heat pump system.

In some embodiments, the present invention is directed to a flooded evaporator chiller utilizing a working fluid comprising an HFO-1234ze(Z) composition according to any of the embodiments disclosed herein. In some embodiments, the present invention is directed to a method for producing cooling in a chiller comprising condensing any of the foregoing compositions in a flooded evaporator.

According to any of the foregoing embodiments, also disclosed herein is a method for replacing a first refrigerant composition with a second refrigerant composition in a cooling or heating system. The method comprises removing the first refrigerant composition from the cooling or heating system and charging the second refrigerant composition to the cooling or heating system. The first refrigerant is selected from the group of HCFO-1233zd(E), HFO-1336mzz(Z) and HFO-1336mzz(E), and the second refrigerant composition is any of the HFO-1234ze(Z) compositions disclosed herein.

The foregoing general description and the following detailed description are exemplary and explanatory only and are not restrictive of the invention, as defined in the appended claims. The various embodiments of the invention can be used alone or in combinations with each other. Other features and advantages of the present invention will be apparent from the following more detailed description of the preferred embodiment, taken in conjunction with the accompanying drawings, which illustrate, by way of example, the principles of the invention.

### BRIEF DESCRIPTION OF THE SEVERAL VIEWS OF THE DRAWINGS

The following detailed description of preferred embodiments of the present invention will be better understood when read in conjunction with the appended drawing. For the purposes of illustrating the invention, there is shown in the drawing an embodiment which is presently preferred. It is understood, however, that the invention is not limited to the precise arrangements and instrumentalities shown. In the drawings:

FIG. 1 is a schematic diagram of one embodiment of a flooded evaporator heat pump apparatus or flooded evaporator chiller which utilizes a composition comprising Z-HFO-1234ze or Z-HFO-1234ze blends described herein as working fluid;

FIG. 2 is a schematic diagram of one embodiment of a direct expansion heat pump apparatus or chiller which utilizes a composition comprising Z-HFO-1234ze or Z-HFO-1234ze blends described herein as working fluid;

FIG. 3 is a schematic diagram of a cascade heat pump system which uses a composition comprising Z-HFO-1234ze or Z-HFO-1234ze blends described herein as working fluid in at least one stage;

FIG. 4 provides a graphical comparison of R-1233zdE and R-1234zeZ in a high temperature heat pump cycle with an 80° C. temperature lift to a 130° C. condensing temperature;

FIG. 5 provides a graphical representation of the relative COP for heating of a composition comprising R-1234zeZ, R-1336mzzE and R-1336mzzZ, according to an embodiment of the present invention and more particularly Example 6, relative to that of incumbent fluid R-1233zdE, given by COP_h/COP_h_ref, where COP_h_ref is the COP for heating of R-1233zdE, using the conditions given in Table 7;

FIG. 6 provides a graphical representation of the deviation of dew point pressure from bubble point pressure at the evaporator temperature for a binary composition of R-1234zeZ/R-1336mzzE of an example of an embodiment of the present invention;

FIG. 7 provides a graphical representation of the relative COP for heating of a composition comprising R-1234zeZ, R-1336mzzE and R-245fa, according to an embodiment of the present invention and more particularly Example 7, relative to that of incumbent fluid R-1233zdE, given by COP_h/COP_h_ref, where COP_h_ref is the COP for heating of R-1233zdE, using the conditions given in Table 7; and

FIG. 8 provides a graphical representation of the relative COP for heating of a composition comprising R-1234zeZ, R-1336mzzZ and R-1224ydZ, according to an embodiment of the present invention and more particularly Example 12, relative to that of incumbent fluid R-1233zdE, given by COP_h/COP_h_ref, where COP_h_ref is the COP for heating of R-1233zdE, using the conditions given in Table 21.

### DETAILED DESCRIPTION OF THE INVENTION

### Definitions

A refrigerant is defined as a heat transfer fluid that undergoes a phase change from liquid to gas and back again during a cycle used to transfer of heat.

A heat transfer system is the system (or apparatus) used to produce a heating or cooling effect in a particular space. A heat transfer system may be a mobile system or a stationary system.

Examples of heat transfer systems are any type of refrigeration systems and air conditioning systems including, but are not limited to, stationary heat transfer systems, air conditioners, freezers, refrigerators, heat pumps, flooded evaporator heat pumps, direct expansion chillers heat pumps, chillers, flooded evaporator chillers, direct expansion chillers, walk-in coolers, mobile refrigerators, mobile heat transfer systems, mobile heat pumps, mobile air conditioning units, dehumidifiers, and combinations thereof.

Refrigeration capacity (also referred to as cooling capacity) is a term which defines the change in enthalpy of a refrigerant in an evaporator per pound of refrigerant circulated, or the heat removed by the refrigerant in the evaporator per unit volume of refrigerant vapor exiting the evaporator (volumetric capacity). The refrigeration capacity is a measure of the ability of a refrigerant or heat transfer composition to produce cooling. Therefore, the higher the capacity, the greater the cooling that is produced. Cooling rate refers to the heat removed by the refrigerant in the evaporator per unit time.

Coefficient of performance (COP) is the amount of heat removed divided by the required energy input to operate the cycle. The higher the COP, the higher is the energy efficiency. COP is directly related to the energy efficiency ratio (EER) that is the efficiency rating for refrigeration or air conditioning equipment at a specific set of internal and external temperatures.

As used herein, a working fluid is a composition comprising a compound or mixture of compounds that primarily function to transfer heat from one location at a lower temperature (e.g., an evaporator) to another location at a higher temperature (e.g., a condenser) in a cycle wherein the working fluid undergoes a phase change from a liquid to a vapor, is compressed and is returned back to liquid through cooling of the compressed vapor in a repeating cycle. The cooling of a vapor compressed above its critical point can return the working fluid to a liquid state without condensation. The repeating cycle may take place in systems such as heat pumps, refrigeration systems, refrigerators, freezers, air conditioning systems, air conditioners, chillers, and the like. Working fluids may be a portion of formulations used within the systems. The formulations may also contain other chemical components (e.g., additives) such as those described below.

The term “subcooling” refers to the reduction of the temperature of a liquid below that liquid's saturation point for a given pressure. The saturation point is the temperature at which the vapor is completely condensed to a liquid, but subcooling continues to cool the liquid to a lower temperature liquid at the given pressure. By cooling a liquid below the saturation temperature (or bubble point temperature), the net refrigeration capacity can be increased. Subcooling thereby improves refrigeration capacity and energy efficiency of a system. Subcool amount is the amount of cooling below the saturation temperature (in temperature units).

Superheat is a term that defines how far above its saturation vapor temperature (the temperature at which, if the composition is cooled, the first drop of liquid is formed, also referred to as the “dew point”) a vapor composition is heated. By heating a vapor above the saturation point, the likelihood of condensation upon compression is minimized, and thus superheating minimizes the risk of liquid entering the compressor. The superheat can also contribute to the cycle's cooling and heating capacity.

Temperature glide (sometimes referred to simply as “glide”) is the absolute value of the difference between the starting and ending temperatures of a phase-change process by a refrigerant within a component of a refrigerant system, exclusive of any subcooling or superheating. This term may be used to describe condensation or evaporation of a zeotropic composition. When referring to the temperature glide of a refrigeration, air conditioning or heat pump system, it is common to provide the average temperature glide being the average of the temperature glide in the evaporator and the temperature glide in the condenser.

The net refrigeration effect is the quantity of heat that each kilogram of refrigerant absorbs in the evaporator to produce useful cooling.

The mass flow rate is the quantity of refrigerant in kilograms circulating through the refrigeration, heat pump or air conditioning system over a given period of time.

As used herein, the term “lubricant” means any material added to a composition or a compressor (and in contact with any heat transfer composition in use within any heat transfer system) that provides hydrodynamic lubrication to the compressor to aid in preventing parts from seizing.

Flammability is a term used to mean the ability of a composition to ignite and/or propagate a flame. For refrigerants and other heat transfer compositions, the lower flammability limit (“LFL”) is the minimum concentration of the heat transfer composition in air that is capable of propagating a flame through a homogeneous mixture of the composition and air under test conditions specified in ASTM (American Society of Testing and Materials) E681. The upper flammability limit (“UFL”) is the maximum concentration of the heat transfer composition in air that is capable of propagating a flame through a homogeneous mixture of the composition and air under the same test conditions. Determination of whether a refrigerant compound or mixture able to propagate a flame or not is also done by testing under the conditions of ASTM E-681.

During a refrigerant leak, the more volatile components of a mixture may leak preferentially. Thus, the composition in the system as well as the vapor leaking can vary over the time period of the leak. Thus, a non-flammable mixture may become able to propagate a flame under leakage scenarios. In order to be classified as non-flammable by ASHRAE (American Society of Heating, Refrigeration and Air-conditioning Engineers), a refrigerant or heat transfer composition must be non-flammable as formulated, but also under leakage conditions.

Global warming potential (GWP) is an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100-year time horizon is commonly the value referenced. For mixtures, a weighted average can be calculated based on the individual GWPs for each component.

Ozone depletion potential (ODP) is a number that refers to the amount of ozone depletion caused by a substance. The ODP is the ratio of the impact on ozone of a chemical compared to the impact of a similar mass of CFC-11 (fluorotrichloromethane). Thus, the ODP of CFC-11 is defined to be 1.0. Other CFCs and HCFCs have ODPs that range from 0.01 to 1.0. HFCs and HFOs have zero ODP because they do not contain chlorine or other ozone depleting halogens.

An azeotropic composition may refer to a constant-boiling mixture of two or more substances that behave as a single substance. One way to characterize an azeotropic composition is that the vapor produced by partial evaporation or distillation of the liquid has the same composition as the liquid from which it is evaporated or distilled. For example, the mixture distills/refluxes without compositional change. Constant-boiling compositions are characterized as azeotropic because they exhibit either a maximum or minimum boiling point, as compared with that of the non-azeotropic mixture of the same compounds. An azeotropic composition will not fractionate within a refrigeration or air conditioning system during operation. Additionally, an azeotropic composition will not fractionate upon leakage from a refrigeration or air conditioning system.

By “azeotrope-like” or “azeotropic-like” composition (sometimes referred to as “near-azeotrope”) is meant essentially constant boiling, or substantially constant boiling, liquid admixture of two or more substances that behaves as a single substance. One way to characterize an azeotrope-like composition is that the vapor produced by partial evaporation or distillation of the liquid has substantially the same composition as the liquid from which it was evaporated or distilled, that is, the admixture distills/refluxes without substantial composition change. Another way to characterize an azeotrope-like composition is that the bubble point vapor pressure and the dew point vapor pressure of the composition at a particular temperature are substantially the same, for example within 3 percent. Another way to characterize an azeotrope-like composition is that the difference between the bubble point pressure (“BP”) and dew point pressure (“DP”) of the composition at a particular temperature is less than or equal to 5 percent based upon the bubble point pressure, i.e., [(BP−VP)/BP]×100≤5.

Another manner to characterize a near-azeotropic composition is that the bubble point vapor pressure and the dew point pressure of the composition at a particular temperature are substantially the same. Herein, a composition of the invention is near-azeotropic if, after 50 weight percent (50 wt. %) of the composition is removed, such as by evaporation or boiling off, the difference in vapor pressure, between the original composition and the composition remaining after 50 weight percent of the original composition has been removed, is less than about 10 percent (10%).

An azeotrope-like composition can also be characterized by the area that is adjacent to the maximum or minimum bubble-point pressure in a plot of composition vapor pressure at a given temperature as a function of mole fraction of components in the composition.

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus.

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed provided that these additional included materials, steps, features, components, or elements do not materially affect the basic and novel characteristic(s) of the claimed invention. The term ‘consisting essentially of’ occupies a middle ground between “comprising” and ‘consisting of’. Typically, components of the refrigerant mixtures and the refrigerant mixtures themselves can contain minor amounts (e.g., less than about 0.5 weight percent total) of impurities and/or byproducts (e.g., from the manufacture of the refrigerant components or reclamation of the refrigerant components from other systems) which do not materially affect the novel and basic characteristics of the refrigerant mixture.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the disclosed compositions, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

### Compositions

In one embodiment, the present disclosure provides a composition comprising Z-1,3,3,3-tetrafluoropropene (HFO-1234ze(Z), R-1234ze(Z)). In one embodiment, the compositions according to the present invention comprise HFO-1234ze(Z) and at least one additional compound selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; or at least two additional compounds or at least three additional compounds or more.

In one embodiment, the present disclosure provides a composition comprising Z-1,3,3,3-tetrafluoropropene (HFO-1234ze(Z)) and at least one additional compound selected from HFO-1234ze(E), HFC-1233xf, HCFC-243fa, HCFC-243fb, HFC-245fa, HFO-1233zd(E), HFO-1233zd(Z) and HCFC-244fa, and the total amount of the additional members is greater than 0 and less than about 2 weight percent, greater than 0 and less than about 1 weight percent, greater than 0 and less than about 0.5 weight percent, greater than 0 and less than or equal to about 0.1 weight percent, or at least two additional members or at least three additional members or more.

In one embodiment, the compositions of the present invention comprise HFO-1234ze(Z) and one additional compound, or two additional compounds, or three or more additional compounds.

In some embodiments, certain precursor compounds to HFO-1234ze(Z) contain compounds that then appear as additional compounds in the HFO-1234ze(Z) compositions. In other embodiments, these precursor compounds may themselves react during the HFO-1234ze(Z) formation to produce additional compounds that then appear in the HFO-1234ze(Z) compositions. In other embodiments, the reaction conditions under which the HFO-1234ze(Z) is produced also produce by-products, by which is meant adventitious reaction pathways may occur simultaneously to produce compounds other than HFO-1234ze(Z) and the quantity and identity of these additional compounds will depend upon the particular conditions under which the HFO-1234ze(Z) is produced.

Certain of the additional compounds making up the compositions according to the present invention are defined in Table 1.

TABLE 1

| Name | Structure | Chemical Name |
|---|---|---|
| HFO-1234ze(E) | E(trans)-CF3CH═CHF | E-1,3,3,3-tetrafluoropropene |
| HFO-1234zc | CHF2CH═CF2 | 1,1,3,3-tetrafluoro-1-propene |
| HCFO-1233zd | E- and/or Z-CF3CH═CHCl | 1-chloro-3,3,3-trifluoropropene |
| HFC-245fa | CF3CH2CHF2 | 1,1,1,3,3-pentafluoropropane |
| HFC-263fb | CF3CH2CH3 | 1,1,1-trifluoropropane |
| HCFO-1233xf | CF3CCl═CH2 | 2-chloro-3,3,3-trifluoropropene |
| HCFC-124 | CF3CFHCl | 1-Chloro-1,2,2,2-tetrafluoroethane |
| HCC-40 | CH3Cl | Chloromethane |
| CFC-114 | C2Cl2F4 | 1,2-Dichlorotetrafluoroethane |
| HCFO-1131(E) | CHCl═CHF | trans-1-chloro-2-fluoroethylene |
| CFC-114a | CF3CFCl2 | 1,1-dichloro-1,2,2,2-tetrafluoroethane |
| HCFC-124a | CF2ClCF2H | 1-chloro-1,1,2,2-tetrafluoroethane |
| HFC-227ca | CF3CF2CHF2 | 1,1,1,2,2,3,3-heptafluoro-propane |
| HFO-1234yf | CF3CF═CH2 | 2,3,3,3-tetrafluoro-1-propene |
| HFC-152a | CHF2CH3 | 1,1-difluoroethane |
| HFO-1243zf | C3H3F3 | 3,3,3-trifluoropropene |
| HFC-245cb | CF3CF2CH3 | 1,1,1,2,2-pentafluoropropane |
| HCC-30 | CH2Cl2 | Dichloromethane (Methylene chloride) |
| HFC-134a | CH2F—CF3 | 1,1,1,2-tetrafluoroethane |
| HFC-236fa | CF3—CH2—CF3 | 1,1,1,3,3,3-Hexafluoropropane |
| HFO-1327 | E- and/or Z-C4HF7 | 1,1,1,2,4,4,4-heptafluoro-1-butene |
| HFO-1336mzz(E) | E CF3—CH═CH—CF3 | E-1,1,1,4,4,4-hexafluoro-2-butene |
| CFO-1112a | E- and/or Z-CClF═CClF | 1,2-dichloro-1,2-difluoroethylene |
| HFC-227ea | CF3—CHF—CF3 | 1,1,1,2,3,3,3-heptafluoropropane |

The additional compounds listed in Table 1 are available commercially or can be made by processes known in the art. For example, such compounds can be purchased from a specialty fluorochemical supplier, such as SynQuest Laboratories, Inc. (Alachua, Florida, USA).

In one embodiment, the compositions of the present invention comprise at least about 98% by weight, at least about 99% by weight, at least 99.5% by weight, at least 99.6% by weight, at least 99.7% by weight, at least 99.8% by weight, or about 99.9% by weight of HFO-1234ze(Z) and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb and mixtures thereof.

In one embodiment, for any of the foregoing compositions, the total amount of additional compound(s) in the composition comprising HFO-1234ze(Z) ranges from greater than 0 wt. % to less than or equal to about 2 wt. %, about 1 wt. %, about 0.9 wt. %, about 0.8 wt. %, about 0.7 wt. %, about 0.6 wt. %, about 0.5 wt. %, about 0.4 wt. %, about 0.3 wt. %, about 0.2 wt. %, about 0.1 wt. %, based on the total weight of the composition. In another embodiment, the total amount of additional compound(s) ranges from 0.01 ppm (weight) to about 1 wt. %, and all values therebetween up to 1 wt. %. In another embodiment, the total amount of additional compound(s) ranges from 0.1 ppm (weight) to about 1 wt. %. In another embodiment, the total amount of additional compound(s) ranges from 0.001 wt. % to about 1 wt. %. In another embodiment, the total amount of additional compound(s) ranges from 0.001 wt. % to about 0.5 wt. %. In another embodiment, the total amount of additional compound(s) ranges from 0.001 wt. % to 0.4 wt. % or less, based on the total weight of the composition. In another embodiment, the total amount of additional compound(s) ranges from 0.001 wt. % to 0.1 wt. % or less, based on the total weight of the composition. In one embodiment, the total amount of additional compound(s) is about 0.1 wt. % based on the total weight of the composition.

In one embodiment, the compositions comprise at least about 99% by weight, in some cases at least about 99.5% by weight, of HFO-1234ze(Z) and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb and mixtures thereof, wherein the total amount of the additional compound(s) is about 1% by weight or less, or about 0.5% by weight or less, or about 0.4% by weight or less, or about 0.3% by weight or less, or about 0.2% by weight or less, or about 0.1% by weight or less, based on the total weight of the composition.

In one embodiment, for any of the compositions disclosed herein, the amount of the additional compound HFO-1234ze(E) can range from about 1 ppm to about 500 ppm by weight, about 100 to about 375 ppm, and preferably about 150 ppm to about 250 ppm.

In one embodiment, for any of the compositions disclosed herein, the amount of the additional compound HCFO-1233zd (E isomer) can range from about 1 ppm to about 3,750 ppm by weight, about 750 ppm to about 2,800 ppm, and preferably about 1,125 ppm to about 1,875 ppm.

In one embodiment, for any of the compositions disclosed herein, the amount of the additional compound HFC-245fa can range from about 1 ppm to about 2,500 ppm by weight, about 500 ppm to about 1,875 ppm, and preferably about 750 to about 1,250 ppm.

In one embodiment, for any of the compositions disclosed herein, the amount of the additional compound HCFO-1233xf can range from about 1 ppm to about 1,250 ppm by weight, about 250 to about 950 ppm, and preferably about 375 to about 625 ppm.

In one embodiment, for any of the compositions disclosed herein, the amount of the additional compound HCFC-124a can range from about 1 ppm to about 500 ppm by weight, about 100 to about 375 ppm, and preferably about 150 to about 250 ppm.

In one embodiment, for any of the compositions disclosed herein, the amount of the additional compound HCFO-1233zd (Z isomer) can range from about 1 to about 500 ppm by weight, about 100 to about 375 ppm, and preferably about 150 to about 250 ppm.

In one embodiment, for any of the foregoing compositions, the amount of the additional compound HCFC-124 can range from about 1 ppm to about 250 ppm by weight, about 50 to about 200 ppm, and preferably about 75 to about 125 ppm.

In one embodiment, for any of the compositions disclosed herein, the amount of the additional compound CFC-114a can range from about 1 ppm to about 250 ppm by weight, about 50 to about 200 ppm, and preferably about 75 to about 125 ppm.

In one embodiment, for any of the compositions disclosed herein, the amount of the additional compound HFC-263fb can range from about 0.5 to about 100 ppm by weight, about 3 to about 50 ppm, and preferably about 4 to about 8 ppm.

In one embodiment, for any of the compositions disclosed herein, the amount of the additional compound HFO-1234zc can range from about 0.5 ppm to about 160 ppm by weight, about 30 ppm to about 120 ppm, and preferably about 45 ppm to about 80 ppm.

In one embodiment, for any of the compositions disclosed herein, the amount of the additional compound HCC-40 can range from about 1 ppm to about 50 ppm by weight, about 1 ppm to about 4 ppm, and preferably about 1.5 to about 2.5 ppm.

In one embodiment, for any of the compositions disclosed herein, the amount of CFC-114 can range from about 1 ppm to about 125 ppm by weight, about 25 to about 100 ppm, and preferably about 35 to about 65 ppm.

In one embodiment, for any of the compositions disclosed herein, the amount of the additional compound HCFC-1131 (E isomer) can range from about 1 ppm to about 50 ppm by weight, about 1 ppm to about 4 ppm, and preferably about 1.5 to about 2.5 ppm.

In one embodiment, for any of the compositions disclosed herein, the total amount of one or more additional compounds selected from HFC-227ca, HFO-1234yf, HFC-152a, HFO-1243zf and HFC-245cb can range from about 10 ppm to about 500 ppm, about 20 ppm to about 90 ppm, and preferably about 50 ppm to about 85 ppm.

It will be readily understood by those skilled in the art that the additional compounds present in the compositions and respective amounts of each additional compound which is present will depend upon the method of manufacture and/or parameters thereof.

For the sake of example only, one embodiment of the compositions according to the present invention comprises “neat” HFO-1234ze(Z) and a plurality of additional compounds in the amounts shown in Table 2 totaling less than about 0.4% by weight. It will be understood that the total amount of the additional compounds may be less than about 0.5% by weight, less than about 0.4% by weight, less than about 0.3% by weight, less than about 0.2% by weight or less than about 0.1% by weight.

TABLE 2
Exemplary Composition of One Embodiment

| Column | ppm |
|---|---|
| 1234ze-E | 200.00 |
| 263fb | 6.36 |
| 1234zc | 63.65 |
| 245fa | 1000.00 |
| 1233zd-E | 1500.00 |
| 1234ze-Z | 996000.00 |
| 1233zd-Z | 200.00 |
| 1233xf | 500.00 |
| 124 | 100.00 |
| 40 | 2.00 |
| 114 | 50.00 |
| 1131-E | 2.00 |
| 114a | 100.00 |
| 124a | 200.00 |
| Others | 75.98 |

In Table 2, “Others” represents a combined total amount of HFC-227ca, HFO-1234yf, HFC-152a, HFO-1243zf and HFC-245cb.

### Blend Compositions

In another embodiment, blend compositions of the present invention comprise HFO-1234ze(Z) and at least one compound selected from HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224yd(E), CFO-1112(E), CFO-1112(Z), HFC-245fa, HFC-236fa, HFC-227ea, trans-1,2-dichloroethylene, HFO-1132(E), HFO-1132(Z), HFC-152a, HFC-134a, HFC-134, HFC-32, HFC-125, carbon dioxide, isobutene, propane, butane, isobutane, pentane and isopentane.

In another embodiment, blend compositions of the present invention comprise HFO-1234ze(Z) and at least one compound selected from HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224yd(E), CFO-1112(E), CFO-1112(Z), HFC-245fa, HFC-236fa, HFC-227ea, trans-1,2-dichloroethylene, HFO-1132(E), HFO-1132(Z), HFC-152a, HFC-134a, HFC-134, HFC-32, HFC-125, carbon dioxide, isobutene, propane, butane, isobutane, pentane and isopentane, and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In another embodiment, blend compositions of the present invention comprise HFO-1234ze(Z) and at least one compound selected from HFO-1336mzz(Z), HFO-1336mzz(E), HFO-1234ze(E), HCFO-1233zd(E), HFC-245fa, isobutane, trans-1,2-dichloroethene, HCFO-1224 yd(Z), HCFO-1224yd(E), HFC-134 and HFC-227ea.

In another embodiment, blend compositions of the present invention comprise HFO-1234ze(Z) and at least one compound selected from HFO-1336mzz(Z), HFO-1336mzz(E), HFO-1234ze(E), HCFO-1233zd(E), HFC-245fa, isobutane, trans-1,2-dichloroethene, HCFO-1224 yd(Z), HCFO-1224yd(E), HFC-134 and HFC-227ea, and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In another embodiment, the present invention is directed to a composition comprising a blend comprising Z-1,3,3,3-tetrafluoropropene and at least one compound selected from HFO-1234yf, HFO-1234ze(E), HFO-1225ye, HFO-1336mzz(Z), HFO-1336mzz(E), HFO-1336yf, HCFO-1336ze(E), HCFO-1336ze(Z), HCFO-1233zd(E), HCFO-1233zd(Z), HCFO-1224 yd(Z), HCFO-1224yd(E), E-CFO-1112, Z-CFO-1112, HFC-227ea, HFO-1132(E), HFO-1132(Z), trans-1,2-dichloroethylene, HFC-152a, HFC-134a, HFC-134, HFC-32, HFC-125.

In another embodiment, the present invention is directed to a composition comprising a blend comprising Z-1,3,3,3-tetrafluoropropene and at least one compound selected from HFO-1234yf, HFO-1234ze(E), HFO-1225ye, HFO-1336mzz(Z), HFO-1336mzz(E), HFO-1336yf, HCFO-1336ze(E), HCFO-1336ze(Z), HCFO-1233zd(E), HCFO-1233zd(Z), HCFO-1224 yd(Z), HCFO-1224yd(E), E-CFO-1112, Z-CFO-1112, HFC-227ea, HFO-1132(E), HFO-1132(Z), trans-1,2-dichloroethylene, HFC-152a, HFC-134a, HFC-134, HFC-32, HFC-125, and further comprising at least one additional compound selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In another embodiment, the present invention is directed to a composition comprising a blend comprising Z-1,3,3,3-tetrafluoropropene and at least one compound selected from HFO-1336mzz(Z), HFO-1336mzz(E), HFO-1336yf, HCFO-1336ze(Z), HCFO-1336ze(E), HFO-1234ze(E), HCFO-1233zd(E), HFC-245fa, HFC-236fa, carbon dioxide, isobutene, propane, butane, isobutane, pentane and isopentane.

In another embodiment, the present invention is directed to a composition comprising a blend comprising Z-1,3,3,3-tetrafluoropropene and at least one compound selected from HFO-1336mzz(Z), HFO-1336mzz(E), HFO-1336yf, HCFO-1336ze(E), HCFO-1336ze(Z), HFO-1234ze(E), HCFO-1233zd(E), HFC-245fa, HFC-236fa, carbon dioxide, isobutene, propane, butane, isobutane, pentane and isopentane, and further comprising at least one additional compound selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In another embodiment, the present invention is directed to a composition comprising a blend comprising Z-1,3,3,3-tetrafluoropropene and at least one compound selected from HFO-1336mzz(Z), HFO-1336mzz(E), HCFO-1233zd(E), HCFO-1233zd(Z), HFO-1234ze(E), HFC-236fa, HFC-134, HFC-152a, HFC-245fa, HFO-1132(E), and HFO-1132(Z).

In another embodiment, the present invention is directed to a composition comprising a blend comprising Z-1,3,3,3-tetrafluoropropene and at least one compound selected from HFO-1336mzz(Z), HFO-1336mzz(E), HCFO-1233zd(E), HCFO-1233zd(Z), HFO-1234ze(E), HFC-236fa, HFC-134, HFC-152a, HFC-245fa, HFO-1132(E), and HFO-1132(Z), and further comprising at least one additional compound selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In some embodiments, compositions of the present invention comprise HFO-1234ze(Z) and HFO-1336mzz(Z).

In some embodiments, compositions of the present invention comprise HFO-1234ze(Z) and HFO-1336mzz(Z), and further comprise one or more additional compounds selected from HFO-1336mzz(E), HFO-1327mz, HFO-1326mxz(Z), HFO-1326mxz(E), HFC-356mff, CHFC-346mdf, and HFC-263fb, and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In some embodiments, compositions of the present invention comprise Z-1,3,3,3-tetrafluoropropene in an amount of about 0.5 wt % to about 99.5 wt % and HFO-1336mzz(Z) in an amount of about 0.5 wt % to about 99.5 wt %, based on the total composition, with up to about 0.5 wt % containing (i) one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (ii) one or more additional compounds selected from HFO-1336mzz(E), HFO-1327mz, HFO-1326mxz(Z), HFO-1326mxz(E), HFC-356mff, CHFC-346mdf, and HFC-263fb.

In some embodiments, compositions of the present invention comprise Z-1,3,3,3-tetrafluoropropene in an amount of about 72 wt % to about 99.5 wt %, and HFO-1336mzz(Z) in an amount of about 0.5 wt % to about 28 wt %, based on the total composition, with up to about 0.5 wt % containing (i) one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (ii) one or more additional compounds selected from HFO-1336mzz(E), HFO-1327mz, HFO-1326mxz(Z), HFO-1326mxz(E), HFC-356mff, CHFC-346mdf, and HFC-263fb.

In one embodiment, compositions of the present invention comprise about 72 wt % of Z-1,3,3,3-tetrafluoropropene and about 28 wt % of HFO-1336mzz(Z), based on the total composition, with up to about 0.5 wt % containing (i) one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (ii) one or more additional compounds selected from HFO-1336mzz(E), HFO-1327mz, HFO-1326mxz(Z), HFO-1326mxz(E), HFC-356mff, CHFC-346mdf, and HFC-263fb.

In some embodiments, compositions of the present invention comprise HFO-1234ze(Z) and HFO-1336mzz(E).

In some embodiments, compositions of the present invention comprise HFO-1234ze(Z) and HFO-1336mzz(E), and further comprise (i) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and (ii) further comprise one or more additional compounds selected from Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In some embodiments, compositions of the present invention comprise Z-1,3,3,3-tetrafluoropropene in an amount of about 0.5 wt % to about 99.5 wt % and HFO-1336mzz(E) in an amount of about 0.5 wt % to about 99.5 wt %, based on the total composition, with up to about 0.5 wt % containing (i) one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (ii) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf.

In one embodiment, compositions of the present invention comprise Z-1,3,3,3-tetrafluoropropene in an amount of about 29 wt % to about 66 wt %, and HFO-1336mzz(E) in an amount of about 34 wt % to about 71 wt %, based on the total composition, with up to about 0.5 wt % containing (i) one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (ii) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf.

In some embodiments, compositions of the present invention comprise HFO-1234ze(Z), HFO-1336mzz(E) and HFO-1336mzz(Z). In some embodiments, compositions of the present invention comprise Z-1,3,3,3-tetrafluoropropene in an amount of about 0.5 wt % to about 99.5 wt %, HFO-1336mzz(E) in an amount of about 0.5 wt % to about 99.5 wt %, and HFO-1336mzz(Z) in an amount of about 0.5 wt % to about 30 wt %, based on the total composition, with up to about 0.5 wt % containing (i) one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; (ii) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or (iii) one or more additional compounds selected from HFO-1336mzz(E), HFO-1327mz, HFO-1326mxz(Z), HFO-1326mxz(E), HFC-356mff, CHFC-346mdf, and HFC-263fb.

In one embodiment, compositions of the present invention comprise HFO-1234ze(Z), HFO-1336mzz(E) and HFO-1336mzz(Z). In some embodiments, compositions of the present invention comprise Z-1,3,3,3-tetrafluoropropene in an amount of about 27 wt % to about 72 wt %, HFO-1336mzz(E) in an amount of about 0.5 wt % to about 70.6 wt %, and HFO-1336mzz(Z) in an amount of about 0.5 wt % to about 27.9 wt %, based on the total composition, with up to about 0.5 wt % containing (i) one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; (ii) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or (iii) one or more additional compounds selected from HFO-1336mzz(E), HFO-1327mz, HFO-1326mxz(Z), HFO-1326mxz(E), HFC-356mff, CHFC-346mdf, and HFC-263fb.

It was surprisingly found that even a small amount of HFO-1336mzz(E) being present in the composition, together with HFO-1234ze(Z) and HFO-1336mzz(Z), is advantageous in order to avoid wet compression as there is no superheat, and to leverage the superior heating capacity and efficiency achievable by the HFO-1234ze(Z) and HFO-1336mzz(Z).

It was additionally surprising that even a small amount of HFO-1336mzz(E) being present in the composition, together with HFO-1234ze(Z) and HFO-1336mzz(Z), is advantageous in order to 1) reduce the compressor discharge temperature, 2) reduce the compression ratio of the cycle, 3) reduce the heat exchanger glide, 4) reduce the heat of combustion, 5) raise the LFL and 6) raise the heat transfer coefficient for pool boiling in the evaporator.

In some embodiments, compositions of the present invention comprise HFO-1234ze(Z), HFO-1336mzz(E) and HFO-1336mzz(Z), and further comprise (i) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, (ii) one or more additional compounds selected from HFO-1336mzz(E), HFO-1327mz, HFO-1326mxz(Z), HFO-1326mxz(E), HFC-356mff, CHFC-346mdf, and HFC-263fb, and (iii) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In some embodiments, compositions of the present invention comprise HFO-1234ze(Z), HFO-1336mzz(E) and HFC-245fa.

In some embodiments, compositions of the present invention comprise HFO-1234ze(Z), HFO-1336mzz(E) and HFC-245fa, and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and further comprise one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or further comprise one or more additional compounds selected from CFC-141b, HCFC-235fa and HCFC-243fa.

In some embodiments, compositions of the present invention comprise Z-1,3,3,3-tetrafluoropropene in an amount of about 1 wt % to about 99 wt %, HFO-1336mzz(E) in an amount of about 1 wt % to about 99 wt %, and HFC-245fa in an amount of about 0.5 wt % to about 99 wt %, based on the total composition, with up to about 0.5 wt % containing one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from CFC-141b, HCFC-235fa and HCFC-243fa.

In one embodiment, compositions of the present invention comprise Z-1,3,3,3-tetrafluoropropene in an amount of about 10 wt % to about 80 wt %, HFO-1336mzz(E) in an amount of about 5 wt % to about 50 wt %, and HFC-245fa in an amount of about 5 wt % to about 50 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from CFC-141b, HCFC-235fa and HCFC-243fa.

In another embodiment, compositions of the present invention comprise Z-1,3,3,3-tetrafluoropropene in an amount of about 9 wt % to about 66 wt %, HFO-1336mzz(E) in an amount of about 20 wt % to about 71 wt %, and HFC-245fa in an amount of about 0.5 wt % to about 34 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from CFC-141b, HCFC-235fa and HCFC-243fa.

In some embodiments, compositions of the present invention comprise HFO-1234ze(Z), HFO-1336mzz(E) and isobutane.

In some embodiments, compositions of the present invention comprise HFO-1234ze(Z), HFO-1336mzz(E) and isobutane, and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and further comprise one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and further comprise one or more additional compounds such as butane.

In some embodiments, compositions of the present invention comprise Z-1,3,3,3-tetrafluoropropene in an amount of about 1 wt % to about 99 wt %, HFO-1336mzz(E) in an amount of about 1 wt % to about 99 wt %, and isobutane in an amount of about 0.1 wt % to about 15 wt %, based on the total composition, with up to about 0.5 wt % containing one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or an additional compound of butane.

In one embodiment, compositions of the present invention comprise Z-1,3,3,3-tetrafluoropropene in an amount of about 10 wt % to about 80 wt %, HFO-1336mzz(E) in an amount of about 10 wt % to about 80 wt %, and isobutane in an amount of about 0.1 wt % to about 2 wt % (or 0.1 wt % to about 10 wt %, 0.1 wt % to about 8 wt %, or 0.1 wt % to about 5 wt %), based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or an additional compound such as butane.

In another embodiment, compositions of the present invention comprise Z-1,3,3,3-tetrafluoropropene in an amount of about 30 wt % to about 80 wt %, HFO-1336mzz(E) in an amount of about 6 wt % to about 70 wt %, and isobutane in an amount of about 0.1 wt % to about 2 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or an additional compound such as butane.

In some embodiments, compositions of the present invention comprise HFO-1234ze(Z), HFO-1336mzz(E) and HFC-227ea.

In some embodiments, compositions of the present invention comprise HFO-1234ze(Z), HFO-1336mzz(E) and HFC-227ea, and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and further comprise one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and further comprise one or more additional compounds selected from FC-1216 and HCFC-124.

In some embodiments, compositions of the present invention comprise Z-1,3,3,3-tetrafluoropropene in an amount of about 1 wt % to about 99 wt %, HFO-1336mzz(E) in an amount of about 1 wt % to about 99 wt %, and HFC-227ea in an amount of about 0.1 wt % to about 15 wt %, based on the total composition, with up to about 0.5 wt % containing one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from FC-1216 and HCFC-124.

In one embodiment, compositions of the present invention comprise Z-1,3,3,3-tetrafluoropropene in an amount of about 10 wt % to about 80 wt %, HFO-1336mzz(E) in an amount of about 10 wt % to about 80 wt %, and HFC-227ea in an amount of about 0.1 wt % to about 10 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from FC-1216 and HCFC-124.

In another embodiment, compositions of the present invention comprise Z-1,3,3,3-tetrafluoropropene in an amount of about 30 wt % to about 68 wt %, HFO-1336mzz(E) in an amount of about 24 wt % to about 70 wt %, and HFC-227ea in an amount of about 0.1 wt % to about 8 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from FC-1216 and HCFC-124.

In some embodiments, compositions of the present invention comprise HFO-1234ze(Z), HFO-1336mzz(E), HFO-1336mzz(Z) and HFC-134.

In some embodiments, compositions of the present invention comprise HFO-1234ze(Z), HFO-1336mzz(E), HFO-1336mzz(Z), and HFC-134, and further comprise (i) one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, (ii) one or more additional compounds selected from HFO-1336mzz(E), HFO-1327mz, HFO-1326mxz(Z), HFO-1326mxz(E), HFC-356mff, CHFC-346mdf, and HFC-263fb, (iii) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and/or (iv) one or more additional compounds selected from HFC-134a, HCFC-124, CHFO-1122, HFC-143a, HFC-32, HFC-125, CFC-114 and CFC-114a.

In some embodiments, compositions of the present invention comprise Z-1,3,3,3-tetrafluoropropene in an amount of about 0.5 wt % to about 99 wt %, HFO-1336mzz(E) in an amount of about 1 wt % to about 99 wt %, HFO-1336mzz(Z) in an amount of about 0.1 wt % to about 99 wt %, and HFC-134 in an amount of about 0.1 wt % to about 40 wt %, based on the total composition, with up to about 0.5 wt % containing one or more of the additional compounds shown of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; one or more additional compounds selected from HFO-1336mzz(E), HFO-1327mz, HFO-1326mxz(Z), HFO-1326mxz(E), HFC-356mff, CHFC-346mdf, and HFC-263fb; and/or one or more additional compounds selected from HFC-134a, HCFC-124, CHFO-1122, HFC-143a, HFC-32, HFC-125, CFC-114 and CFC-114a.

In one embodiment, compositions of the present invention comprise Z-1,3,3,3-tetrafluoropropene in an amount of about 10 wt % to about 80 wt %, HFO-1336mzz(E) in an amount of about 5 wt % to about 70 wt %, HFO-1336mzz(Z) in an amount of about 5 wt % to about 70 wt %, and HFC-134 in an amount of about 0.1 wt % to about 5 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; one or more additional compounds selected from HFO-1336mzz(E), HFO-1327mz, HFO-1326mxz(Z), HFO-1326mxz(E), HFC-356mff, CHFC-346mdf, and HFC-263fb; and/or one or more additional compounds selected from HFC-134a, HCFC-124, CHFO-1122, HFC-143a, HFC-32, HFC-125, CFC-114 and CFC-114a.

In another embodiment, compositions of the present invention comprise Z-1,3,3,3-tetrafluoropropene in an amount of about 0.5 wt % to about 64 wt %, HFO-1336mzz(E) in an amount of about 14 wt % to about 74 wt %, HFO-1336mzz(Z) in an amount of about 0.1 wt % to about 60 wt %, and HFC-134 in an amount of about 0.1 wt % to about 26 wt %, based on the total composition, with up to about 0.5 wt % containing one or more of the additional compounds of Table 2 and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; one or more additional compounds selected from HFO-1336mzz(E), HFO-1327mz, HFO-1326mxz(Z), HFO-1326mxz(E), HFC-356mff, CHFC-346mdf, and HFC-263fb; and/or one or more additional compounds selected from HFC-134a, HCFC-124, CHFO-1122, HFC-143a, HFC-32, HFC-125, CFC-114 and CFC-114a.

In some embodiments, compositions of the present invention comprise HFO-1234ze(Z), HFO-1234ze(E) and HFC-134.

In some embodiments, compositions of the present invention comprise HFO-1234ze(Z), HFO-1234ze(E), and HFC-134, and further comprise (i) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; (ii) one or more additional compounds selected from HFC-134a, HFO-1225zc, HFO-1234yf, HFC-245cb, HFC-236fa, HFO-1225ye(E), HFO-1234zc, HFC-245fa, HCFC-124, HFO-1234ze(Z), CFC-114, trifluoropropyne, HFC-152a, HFO-1225ye (Z), HFO-1225ye(E), HCFO-1233xf, HFC-263fb, HFO-1243zf and HCFO-1233zd(E); and/or (iii) one or more additional compounds selected from HFC-134a, HCFC-124, CHFO-1122, HFC-143a, HFC-32, HFC-125, CFC-114 and CFC-114a.

In some embodiments, compositions of the present invention comprise Z-1,3,3,3-tetrafluoropropene in an amount of about 0.1 wt % to about 99 wt %, HFO-1234ze(E) in an amount of about 0.1 wt % to about 99 wt %, and HFC-134 in an amount of about 0.1 wt % to about 50 wt %, based on the total composition, with up to about 0.5 wt % containing one or more of the above-listed additional compounds.

In some embodiments, compositions of the present invention comprise HFO-1234ze(Z), HFO-1234ze(E), HCFO-1233zd(E) and HFC-134. In some embodiments, compositions of the present invention comprise HFO-1234ze(Z), HFO-1234ze(E), HCFO-1233zdE and HFC-134, and further comprise (i) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; (ii) one or more additional compounds selected from HFC-134a, HFO-1225zc, HFO-1234yf, HFC-245cb, HFC-236fa, HFO-1225ye(E), HFO-1234zc, HFC-245fa, HCFC-124, HFO-1234ze(Z), CFC-114, trifluoropropyne, HFC-152a, HFO-1225ye (Z), HFO-1225ye(E), HCFO-1233xf, HFC-263fb, HFO-1243zf and HCFO-1233zd(E); (iii) one or more additional compounds selected from HFO-1234ze(Z), HCFO-1233zd(Z) and HFC-245fa; and/or (iv) one or more additional compounds selected from HFC-134a, HCFC-124, CHFO-1122, HFC-143a, HFC-32, HFC-125, CFC-114 and CFC-114a.

In some embodiments, compositions of the present invention comprise Z-1,3,3,3-tetrafluoropropene in an amount of about 0.1 wt % to about 99 wt %, HFO-1234ze(E) in an amount of about 0.1 wt % to about 99 wt %, HCFO-1233zd(E) in an amount of about 0.1 wt % to about 99 wt %, and HFC-134 in an amount of about 0.1 wt % to about 50 wt %, based on the total composition, with up to about 0.5 wt % containing one or more of the above-listed additional compounds.

In some embodiments, compositions of the present invention comprise HFO-1234ze(Z), HFO-1336mzz(Z) and HCO-1130(E).

In some embodiments, compositions of the present invention comprise HFO-1234ze(Z), HFO-1336mzz(Z), and HCO-1130(E), and further comprise (i) one or more additional compounds selected from HFO-1336mzz(E), HFO-1327mz, HFO-1326mxz(Z), HFO-1326mxz(E), HFC-356mff, CHFC-346mdf, and HFC-263fb; (ii) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and/or (iii) one or more additional compounds such as HCO-1130(Z).

In some embodiments, compositions of the present invention comprise Z-1,3,3,3-tetrafluoropropene in an amount of about 0.1 wt % to about 99 wt %, HFO-1336mzz(Z) in an amount of about 0.1 wt % to about 99 wt %, HCO-1130(E) in an amount of about 0.1 wt % to about 99 wt %, with up to about 0.5 wt % containing one or more of the above-listed additional compounds.

In some embodiments, compositions of the present invention comprise HFO-1234ze(Z), HFO-1336mzz(Z) and HCFO-1224 yd(Z).

In some embodiments, compositions of the present invention comprise HFO-1234ze(Z), HFO-1336mzz(Z) and HCFO-1224 yd(Z), and further comprise (i) one or more additional compounds selected from HFO-1336mzz(E), HFO-1327mz, HFO-1326mxz(Z), HFO-1326mxz(E), HFC-356mff, CHFC-346mdf, and HFC-263fb; (ii) one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and/or (iii) one or more additional compounds selected from HCFO-1224yd(E), HFO-1234yf, HFC-254eb, HCFC-244bb, CFC-1215yb.

In some embodiments, compositions of the present invention comprise Z-1,3,3,3-tetrafluoropropene in an amount of about 0.1 wt % to about 99 wt %, HFO-1336mzz(Z) in an amount of about 0.1 wt % to about 99 wt %, HCFO-1224 yd(Z) in an amount of about 0.1 wt % to about 99 wt %, with up to about 0.5 wt % containing one or more of the above-listed additional compounds.

The total amount of additional compounds in any of the blend compositions disclosed herein ranges from greater than 0 wt. % to less than or equal to about 2 wt. %, about 1 wt. %, about 0.9 wt. %, about 0.8 wt. %, about 0.7 wt. %, about 0.6 wt. %, about 0.5 wt. %, about 0.4 wt. %, about 0.3 wt. %, about 0.2 wt. %, about 0.1 wt. %, based on the total weight of the composition. In another embodiment, the total amount of additional compound(s) ranges from 0.01 ppm (weight) to about 1 wt. %, and all values therebetween up to 1 wt. %. In another embodiment, the total amount of additional compound(s) ranges from 0.1 ppm (weight) to about 1 wt. %. In another embodiment, the total amount of additional compound(s) ranges from 0.001 wt. % to about 1 wt. %. In another embodiment, the total amount of additional compound(s) ranges from 0.001 wt. % to about 0.5 wt. %. In another embodiment, the total amount of additional compound(s) ranges from 0.001 wt. % to 0.4 wt. % or less, based on the total weight of the composition. In another embodiment, the total amount of additional compound(s) ranges from 0.001 wt. % to 0.1 wt. % or less, based on the total weight of the composition. In one embodiment, the total amount of additional compound(s) is about 0.1 wt. % based on the total weight of the composition.

Some of the compounds making up the blend compositions of the present invention are defined in Table 3.

TABLE 3

| Name | Structure | Chemical name |
|---|---|---|
| HFO-1234ze | E(trans)-CF₃CH═CHF | E-1,3,3,3-tetrafluoropropene |
| HFO-1234yf | CF₃CF═CH₂ | 2,3,3,3-tetrafluoro-1-propene |
| HFO-1225ye | CHF₂CF═CHF | 1,2,3,3-tetrafluoro-1-propene |
| HFO-1336mzz | E- and/or Z-CF₃CH═CF₃CH | 1,1,1,4,4,4-hexafluoro-2-butene |
| HFO-1336yf | CF₃CF₂CF═CH₂ | 2,3,3,4,4,4-hexafluoro-1-butene |
| HFC-1336ze | CHF═CHCF₂CF₃ | 1,3,3,4,4,4-hexafluoro-1-butene |
| HCFO-1233zd | E- and/or Z-CF₃CH═CHCl | 1-chloro-3,3,3-trifluoropropene |
| HCFO-1224yd | E- and/or Z-CF₃CF═CHCl | 1-Chloro 2,3,3,3,-tetrafluoropropene |
| HFO-1132 | E- and/or Z-CHF═CHF | 1,2-Difluoroethylene |
| CFO-1112 | E- and/or Z-CClF═CClF | 1,2-dichloro-1,2-difluoroethylene |
| HFC-245fa | CF₃CH₂CHF₂ | 1,1,1,3,3-pentafluoropropane |
| HFC-236a | CF₃CH₂CF₃ | 1,1,1,2,3,3-Hexafluoropropane |
| HFC-227ea | CF₃CF₂CHF₂ | 1,1,1,2,2,3,3,3-heptafluoropropane |
| Trans-1,2-DCE | ClCH=CHCl | trans-1,2-Dichloroethylene |
| HFC-152a | CF₂HCH₃ | 1,1-Difluoroethane |
| HFC-134a | CF₂HCFH₂ | 1,1,1,2-tetrafluoroethane |
| HFC-32 | CF₂H₂ | Difluoromethane |
| HFC-125 | CF₃CF₂H | Pentafluoroethane |
| Isobutene | (CH₃)₂C=CH₂ | 2-methylpropene |
| Propane | CH₃CH₂CH₃ | — |
| Butane | CH₃CH₂CH₂CH₃ | — |
| Isobutane | CH(CH₃)₃ | — |
| Pentane | CH₃(CH₂)₃CH₃ | — |
| Isopentane | CH(CH₃)₂(CH₂CH₃) | — |
| HFO-1327mz | E- and/or Z-C₄HF₇ | 1,1,1,2,4,4,4-heptafluoro-1-butene |
| HFO-1326mxz | E- and/or Z-CF₃CH═CClCF₃ | 2-chloro-1,1,1,4,4,4-hexafluoro-2-butene |
| HFC-356mff | CF₃CH₂CH₂CF₃ | 1,1,1,4,4,4-hexafluorobutane |
| CHFC-346mdf | CF₃CHClCH₂CF₃ | 2-chloro-1,1,1,1,4,4,4-hexafluorobutane |
| HFC-263fb | CF₃CH₂CH₃ | 1,1,1-trifluoropropane |
| HCFO-1233xf | CF₃CCl═CH₂ | 1,1,1-trifluoro-2-chloropropene |
| HFO-1336ft | CH₂═C(CF₃)₂ | 3,3,3-trifluoro-2- |
| | | (trifluoromethyl)-1-propene |
| HCFC-133a | C₂H₂F₃Cl | 1-chloro-2,2,2-trifluoroethane |
| HCO-1140 | C₂H₃Cl | Chloroethylene (vinyl chloride) |
| HFC-347mef | CF₃CHFCH₂CF₃ | 1,1,1,2,4,4,4-hepafluorobutane |
| HFO-1243zf | CF₃CH═CH₂ | 3,3,3-trifluoro-1-propene |

Certain of the compounds of Tables 1-3 exist as different configurational isomers or stereoisomers. When the specific isomer is not designated, the present invention is intended to include all single configurational isomers, single stereoisomers, single geometric or any combination thereof. For instance, HCFO-1233zd is meant to represent the E-isomer, Z-isomer, or any combination or mixture of both isomers in any ratio. As another example, HFO-1224zb is meant to represent the E-isomer, Z-isomer, or any combination or mixture of both isomers in any ratio.

In accordance with one embodiment of the present invention, the blend compositions may be azeotropic or azeotropic-like. In some embodiments, the blend compositions achieve a glide of less than about 1K.

In some embodiments, the HFO-1234ze(Z) compositions and HFO-1234ze(Z) blend compositions (collectively referred to herein as the “HFO-1234ze(Z) composition” or “HFO-1234ze(Z) compositions”) have a flammability classification of 1, 2 L or 2 as determined by ASHRAE Standard 34 and ASTM E681-09. Preferably, the HFO-1234ze(Z) compositions have a flammability rating of 1 or 2 L, as determined by ASHRAE Standard 34 and ASTM E681-09.

In some embodiments, the HFO-1234ze(Z) compositions have a GWP of less than 700, or less than 300, or less than 150, or less than 75, or less than 5 GWP, and all values and ranges therebetween. Since HFO-1234ze(Z) has a GWP of less than 1, it is possible that the compositions according to the present invention have a GWP of less than 1.

In some embodiments, the HFO-1234ze(Z) compositions according to the present invention and the degradation products thereof are preferably free of or substantially free of Group A Fluorinated Substances.

In one embodiment, as used herein, “Group A Fluorinated Substances” includes any substance that (i) contains at least one fully fluorinated methyl (—CF₃) or methylene (—CF₂—) carbon atom (without any H/Cl/Br/I attached to it); and (ii) meets the criterion for persistence in soil/sediment and water established in Annex XIII (Section 1.1.1) of the European Union's REACH Regulation (https://reachonline.eu/reach/en/annex-xiii-1-1.1-1.1.1.html as accessed on May 2, 2023) and referenced in the Annex XV Restriction Report dated Mar. 22, 2023, the disclosure of which is hereby incorporated by reference (https://echa.europa.eu/documents/10162/f605d4b5-7c17-7414-8823-b49b9fd43aea as accessed on May 2, 2023). In one embodiment, Group A Fluorinated Substances include, but are not limited to, trifluoroacetic acid (TFA).

In another embodiment, as used herein, “Group A Fluorinated Substances” includes any substance that has a Henry's Law constant ≤250 Pa*m³/mol and contains at least one fully fluorinated methyl (˜CF₃) or methylene (˜CF₂—) carbon atom (without any H/Cl/Br/I attached to it). In one embodiment, Group A Fluorinated Substances include, but are not limited to, TFA.

Thus, according to some embodiments, compositions of the present invention comprise HFO-1234ze(Z), as a single fluid or blend, and are free of or substantially free of Group A Fluorinated Substances, such as TFA. In one embodiment, the phrase “free of” as used herein with respect to the presence of Group A Fluorinated Substances in the present compositions means that the amount of such substances in the compositions is sufficiently low so as to not be detectable, including but not limited to 0%, when measured by gas chromatography with a flame ionization detector, gas chromatography with a mass detector by analysis of a gas sample or liquid sample, and/or ion chromatography by analysis of a water sample after bubbling the thermal fluid through water. Such methodologies are well known to those skilled in the art. In one embodiment, the phrase “substantially free of” as used herein with respect to the presence of Group A Fluorinated Substances in the present compositions means that the amount of such substances in the compositions is >0 wt. % and ≤5 wt. %, or >0 wt. % and ≤4 wt. %, or >0 wt. % and ≤3 wt. %, or >0 wt. % and ≤2 wt. %, or >0 wt. % and ≤1 wt. %, and all values and ranges therebetween, when measured by gas chromatographic (GC) techniques, for example gas chromatography (GC) with a flame ionization or electron-capture detector, or GC coupled with a mass detector (gas chromatography/mass spectral (GC/MS) method), by ion chromatograph (IC) or ion chromatography mass spectrometry (IC-MS) techniques, or by high-performance liquid chromatography (HPLC) or high-performance liquid chromatography mass spectrometry (HPLC-MS) techniques. The TFA analytical standard may be used in either gas chromatography or ion chromatography and is available from, for example, Sigma Aldrich.

Further, in some embodiments, degradation products of such compositions of the present invention which comprise HFO-1234ze(Z), as a single fluid or blend, are free of or substantially free of Group A Fluorinated Substances, such as TFA. In one embodiment, the phrase “free of” as used herein with respect to the formation of Group A Fluorinated Substances by the present compositions means that the theoretical molar yield of such substances in environmental compartments of air, soil/sediment and water produced during tropospheric degradation of the compositions is sufficiently low so as to not be detectable, including but not limited to 0%, when measured by GC techniques, for example GC with a flame ionization or electron-capture detector or GC/MS method, by IC or IC-MS techniques, or by HPLC or HPLC-MS techniques. In one embodiment, the phrase “substantially free of” as used herein with respect to the formation of Group A Fluorinated Substances by the present compositions means that the theoretical molar yield of such substances in environmental compartments of air, soil/sediment and water produced during tropospheric degradation of the compositions is >0% and ≤5%, or >0% and ≤4%, or >0% and ≤3%, or >0% and ≤2%, or >0% and ≤1%, and all values and ranges therebetween, when measured by GC techniques, for example GC with a flame ionization or electron-capture detector or GC/MS method, by IC or IC-MS techniques, or by HPLC or HPLC-MS techniques.

In some embodiments, compositions of the present invention comprise, consist of or consist essentially of HFO-1234ze(Z) and further comprise one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and are free of or substantially free of Group A Fluorinated Substances. Further, in some embodiments, degradation products of these compositions are free of or substantially free of Group A Fluorinated Substances.

According to some embodiments, compositions of the present invention comprise HFO-1234ze(Z); one or more compounds selected from HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224 yd(E), CFO-1112(E), CFO-1112 (Z), HFC-245fa, HFC-236fa, HFC-227ea, trans-1,2-dichloroethylene, HFO-1132(E), HFO-1132 (Z), HFC-152a, HFC-134a, HFC-134, HFC-32, HFC-125, carbon dioxide, isobutene, propane, butane, isobutane, pentane and isopentane; and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and are free of or substantially free of Group A Fluorinated Substances. Further, degradation products of some of these compositions are free of or substantially free of Group A Fluorinated Substances.

According to some embodiments, compositions of the present invention comprise HFO-1234ze(Z); one or more compounds selected from HFO-1336mzz(Z), HFO-1336mzz(E), HCFO-1233zd(E), HCFO-1233zd(Z), HFO-1234ze(E), HFC-236fa, HFC-134, HFC-152a, HFC-245fa, HFO-1132(E), and HFO-1132 (Z); and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and are free of or substantially free of Group A Fluorinated Substances, such as TFA. Further, degradation products of these compositions are free of or substantially free of Group A Fluorinated Substances, such as TFA.

In one embodiment, compositions of the present invention comprise, consist of or consist essentially of HFO-1234ze(Z) and HFO-1336mzz(Z), and are free of or substantially free of Group A Fluorinated Substances, such as TFA. Further, degradation products of such HFO-1234ze(Z)/HFO-1336mzz(Z) compositions are free of or substantially free of Group A Fluorinated Substances, such as TFA. In some embodiments, compositions of the present invention comprise HFO-1234ze(Z), HFO-1336mzz(Z), one or more additional compounds selected from HFO-1336mzz(E), HFO-1327mz, HFO-1326mxz(Z), HFO-1326mxz(E), HFC-356mff, CHFC-346mdf, and HFC-263fb, and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and are free of or substantially free of Group A Fluorinated Substances, such as TFA. Further, degradation products of such HFO-1234ze(Z)/HFO-1336mzz(Z) compositions are free of or substantially free of Group A Fluorinated Substances, such as TFA.

In some embodiments, compositions of the present invention comprise, consist of or consist essentially of HFO-1234ze(Z) and HFO-1336mzz(E), and are free of or substantially free of Group A Fluorinated Substances, such as TFA. Further, degradation products of such HFO-1234ze(Z)/HFO-1336mzz(E) compositions are free of or substantially free of Group A Fluorinated Substances, such as TFA. In some embodiments, compositions of the present invention comprise HFO-1234ze(Z), HFO-1336mzz(E), one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and are free of or substantially free of Group A Fluorinated Substances, such as TFA. Further, degradation products of such HFO-1234ze(Z)/HFO-1336mzz(E) compositions are free of or substantially free of Group A Fluorinated Substances, such as TFA.

In some embodiments, compositions of the present invention comprise, consist of or consist essentially of HFO-1234ze(Z), HFO-1336mzz(Z) and HFO-1336mzz(E), and are free of or substantially free of Group A Fluorinated Substances, such as TFA. Further, degradation products of such HFO-1234ze(Z)/HFO-1336mzz(Z)/HFO-1336mzz(E) compositions are free of or substantially free of Group A Fluorinated Substances, such as TFA. In some embodiments, compositions of the present invention comprise HFO-1234ze(Z); HFO-1336mzz(E); HFO-1336mzz(Z); one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; one or more additional compounds selected from HFO-1336mzz(E), HFO-1327mz, HFO-1326mxz(Z), HFO-1326mxz(E), HFC-356mff, CHFC-346mdf, and HFC-263fb; and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and are free of or substantially free of Group A Fluorinated Substances, such as TFA. Further, degradation products of such HFO-1234ze(Z)/HFO-1336mzz(Z)/HFO-1336mzz(E) compositions are free of or substantially free of Group A Fluorinated Substances, such as TFA.

In some embodiments, compositions of the present invention comprise, consist of or consist essentially of HFO-1234ze(Z), HFO-1336mzz(Z) and HFC-245fa, and are free of or substantially free of Group A Fluorinated Substances, such as TFA. Further, degradation products of such HFO-1234ze(Z)/HFO-1336mzz(Z)/HFC-245fa compositions are free of or substantially free of Group A Fluorinated Substances, such as TFA. In some embodiments, compositions of the present invention comprise HFO-1234ze(Z); HFO-1336mzz(Z); HFC-245fa; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and/or one or more additional compounds selected from CFC-141b, HCFC-235fa and HCFC-243fa, and are free of or substantially free of Group A Fluorinated Substances, such as TFA. Further, degradation products of such HFO-1234ze(Z)/HFO-1336mzz(Z)/HFC-245fa compositions are free of or substantially free of Group A Fluorinated Substances, such as TFA.

In some embodiments, compositions of the present invention comprise, consist of or consist essentially of HFO-1234ze(Z), HFO-1336mzz(E) and isobutane, and are free of or substantially free of Group A Fluorinated Substances, such as TFA. In some embodiments, compositions of the present invention comprise HFO-1234ze(Z); HFO-1336mzz(E); isobutane; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and further comprise one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and further comprise one or more additional compounds such as butane, and are free of or substantially free of Group A Fluorinated Substances, such as TFA. Further, degradation products of such HFO-1234ze(Z)/HFO-1336mzz(E)/isobutane compositions are free of or substantially free of Group A Fluorinated Substances, such as TFA.

In some embodiments, compositions of the present invention comprise, consist of or consist essentially of HFO-1234ze(Z), HFO-1336mzz(E) and HFC-227ea, and are free of or substantially free of Group A Fluorinated Substances, such as TFA. In some embodiments, compositions of the present invention comprise HFO-1234ze(Z); HFO-1336mzz(E); HFC-227ea; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and further comprise one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; and further comprise one or more additional compounds selected from FC-1216 and HCFC-124, and are free of or substantially free of Group A Fluorinated Substances, such as TFA. Further, in some embodiments, for example where the concentration of HFC-227ea is relatively low (e.g., less than about 5 wt. %), degradation products of such HFO-1234ze(Z)/HFO-1336mzz(E)/HFC-227ea compositions may be substantially free of Group A Fluorinated Substances, such as TFA.

In some embodiments, compositions of the present invention comprise, consist of or consist essentially of HFO-1234ze(Z), HFO-1336mzz(Z), HFO-1336mzz(E) and HFC-134, and are free of or substantially free of Group A Fluorinated Substances, such as TFA. Further, degradation products of such HFO-1234ze(Z)/HFO-1336mzz(Z)/HFO-1336mzz(E)/HFC-134 compositions are free of or substantially free of Group A Fluorinated Substances, such as TFA. In some embodiments, compositions of the present invention comprise HFO-1234ze(Z); HFO-1336mzz(E); HFO-1336mzz(Z); HFC-134; one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; one or more additional compounds selected from HFO-1336mzz(E), HFO-1327mz, HFO-1326mxz(Z), HFO-1326mxz(E), HFC-356mff, CHFC-346mdf, and HFC-263fb; and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and/or one or more additional compounds selected from HFC-134a, HCFC-124, CHFO-1122, HFC-143a, HFC-32, HFC-125, CFC-114 and CFC-114a, and are free of or substantially free of Group A Fluorinated Substances, such as TFA. Further, degradation products of such HFO-1234ze(Z)/HFO-1336mzz(Z)/HFO-1336mzz(E)/HFC-134 compositions are free of or substantially free of Group A Fluorinated Substances, such as TFA.

In some embodiments, compositions of the present invention comprise, consist of or consist essentially of HFO-1234ze(Z), HFO-1234ze(E) and HFC-134, and are free of or substantially free of Group A Fluorinated Substances, such as TFA. Further, degradation products of such HFO-1234ze(Z)/HFO-1234ze(E)/HFC-134 compositions are free of or substantially free of Group A Fluorinated Substances, such as TFA. In some embodiments, compositions of the present invention comprise HFO-1234ze(Z); HFO-1234ze(E); HFC-134; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HFC-134a, HFO-1225zc, HFO-1234yf, HFC-245cb, HFC-236fa, HFO-1225ye(E), HFO-1234zc, HFC-245fa, HCFC-124, HFO-1234ze(Z), CFC-114, trifluoropropyne, HFC-152a, HFO-1225ye(Z), HFO-1225ye(E), HCFO-1233xf, HFC-263fb, HFO-1243zf and HCFO-1233zd(E); and/or one or more additional compounds selected from HFC-134a, HCFC-124, CHFO-1122, HFC-143a, HFC-32, HFC-125, CFC-114 and CFC-114a, and are free of or substantially free of Group A Fluorinated Substances, such as TFA. Further, degradation products of such HFO-1234ze(Z)/HFO-1234ze(E)/HFC-134 compositions are free of or substantially free of Group A Fluorinated Substances, such as TFA.

In some embodiments, compositions of the present invention comprise, consist of or consist essentially of HFO-1234ze(Z), HFO-1234ze(E), HCFO-1233zd(E) and HFC-134, and are free of or substantially free of Group A Fluorinated Substances, such as TFA. Further, degradation products of such HFO-1234ze(Z)/HFO-1234ze(E)/HCFO-1233zd (E)/HFC-134 compositions are free of or substantially free of Group A Fluorinated Substances, such as TFA. In some embodiments, compositions of the present invention comprise HFO-1234ze(Z); HFO-1234ze(E); HCFO-1233zd(E); HFC-134; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; one or more additional compounds selected from HFC-134a, HFO-1225zc, HFO-1234yf, HFC-245cb, HFC-236fa, HFO-1225ye(E), HFO-1234zc, HFC-245fa, HCFC-124, HFO-1234ze(Z), CFC-114, trifluoropropyne, HFC-152a, HFO-1225ye(Z), HFO-1225ye(E), HCFO-1233xf, HFC-263fb, HFO-1243zf and HCFO-1233zd(E); one or more additional compounds selected from HFO-1234ze(Z), HCFO-1233zd(Z) and HFC-245fa; and/or one or more additional compounds selected from HFC-134a, HCFC-124, CHFO-1122, HFC-143a, HFC-32, HFC-125, CFC-114 and CFC-114a, and are free of or substantially free of Group A Fluorinated Substances, such as TFA. Further, degradation products of such HFO-1234ze(Z)/HFO-1234ze(E)/HCFO-1223zd(E)/HFC-134 compositions are free of or substantially free of Group A Fluorinated Substances, such as TFA.

In some embodiments, compositions of the present invention comprise, consist of or consist essentially of HFO-1234ze(Z), HFO-1336mzz(Z) and HCO-1130(E), and are free of or substantially free of Group A Fluorinated Substances, such as TFA. In some embodiments, compositions of the present invention comprise HFO-1234ze(Z); HFO-1336mzz(Z); HCO-1130(E); one or more additional compounds selected from HFO-1336mzz(E), HFO-1327mz, HFO-1326mxz(Z), HFO-1326mxz(E), HFC-356mff, CHFC-346mdf, and HFC-263fb; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and/or one or more additional compounds such as HCO-1130 (Z), and are free of or substantially free of Group A Fluorinated Substances, such as TFA. Further, degradation products of such HFO-1234ze(Z)/HFO-1234ze(E)/HFO-1336mzz(Z)/HCO-1130(E) compositions are free of or substantially free of Group A Fluorinated Substances, such as TFA.

In some embodiments, compositions of the present invention comprise, consist of or consist essentially of HFO-1234ze(Z), HFO-1336mzz(Z) and HCFO-1224 yd(Z), and are free of or substantially free of Group A Fluorinated Substances, such as TFA. In some embodiments, compositions of the present invention comprise HFO-1234ze(Z); HFO-1336mzz(Z); HCFO-1224 yd(Z); one or more additional compounds selected from HFO-1336mzz(E), HFO-1327mz, HFO-1326mxz(Z), HFO-1326mxz(E), HFC-356mff, CHFC-346mdf, and HFC-263fb; one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and/or one or more additional compounds selected from HCFO-1224 yd(E), HFO-1234yf, HFC-254eb, HCFC-244bb, CFC-1215yb, and are free of or substantially free of Group A Fluorinated Substances, such as TFA. Further, in some embodiments, for example where the concentration of HCFO-1224 yd(Z) is relatively low (e.g., less than about 5 wt. %), degradation products of such HFO-1234ze(Z)/HFO-1336mzz(Z)/HCFO-1224 yd(Z) compositions may be substantially free of Group A Fluorinated Substances, such as TFA.

The compositions of the present invention may be prepared by any convenient method to combine the desired amount of the individual components. A preferred method is to weigh the desired component amounts and thereafter combine, mix or blend the components in an appropriate vessel. Agitation may be used, if desired.

In another embodiment, the compositions disclosed herein may be prepared by a method comprising (i) reclaiming a volume of one or more components of the refrigerant compositions disclosed herein from at least one refrigerant container, (ii) removing impurities sufficiently to enable reuse of said one or more of the reclaimed components, (iii) and optionally, combining all or part of said reclaimed volume of components with at least one additional refrigerant composition or component in order to produce a composition described in the various embodiments herein.

A refrigerant container may be any container in which is stored a composition according to the present invention that has been used in a refrigeration apparatus, air-conditioning apparatus or heat pump apparatus. Said container may be the refrigeration apparatus, air-conditioning apparatus or heat pump apparatus in which the refrigerant composition was used. Additionally, the container may be a storage container for collecting reclaimed refrigerant components, including but not limited to pressurized gas cylinders.

Residual refrigerant means any amount of refrigerant or refrigerant blend component that may be moved out of the refrigerant container by any method known for transferring refrigerant blends or refrigerant blend components.

Impurities may be any component that is in the refrigerant or refrigerant blend component due to its use in a refrigeration apparatus, air-conditioning apparatus or heat pump apparatus. Such impurities include but are not limited to refrigeration lubricants, being those described earlier herein, particulates including but not limited to metal, metal salt or elastomer particles, that may have come out of the refrigeration apparatus, air-conditioning apparatus or heat pump apparatus, and any other contaminants that may adversely affect the performance of the refrigerant composition.

Such impurities may be removed sufficiently to allow reuse of the refrigerant or refrigerant blend component without adversely affecting the performance or equipment within which the refrigerant or refrigerant blend component will be used.

It may be necessary to provide additional refrigerant or refrigerant blend component to the residual refrigerant or refrigerant blend component in order to produce a composition that meets the specifications required for a given product. For instance, if a refrigerant blend has 3 components in a particular weight percentage range, it may be necessary to add one or more of the components in a given amount in order to restore the composition to within the specification limits.

### Further Compositions

In one embodiment, compositions of the present invention may further comprise at least one non-refrigerant component. That is, in one embodiment, the present invention relates to compositions comprising a refrigerant composition, such as any of the compositions comprising HFO-1234ze(Z) disclosed herein, and one or more non-refrigerant components.

The optional non-refrigerant components (also referred to herein as “additives”) in the compositions disclosed herein may include one or more of the following components: lubricants, dyes (including UV dyes), solubilizing agents, compatibilizers, stabilizers, tracers, perfluoropolyethers, anti-wear agents, extreme pressure agents, corrosion and oxidation inhibitors, polymerization inhibitors, metal surface energy reducers, metal surface deactivators, acid scavengers, foam control agents, viscosity index improvers, pour point depressants, detergents, viscosity adjusters, performance enhancers, flame suppressants and mixtures thereof. Indeed, many of these optional non-refrigerant components fit into one or more of these categories and may have qualities that lend themselves to achieve one or more performance characteristics.

The additive component(s) and amounts thereof selected for the disclosed compositions are elected on the basis of utility, individual equipment components, and/or the system requirements.

Lubricants which may be included in compositions of the present invention comprise those suitable for use with refrigeration or air-conditioning apparatus. Among these lubricants are those conventionally used in compression refrigeration apparatus utilizing chlorofluorocarbon refrigerants. Such lubricants and their properties are discussed in the 1990 ASHRAE Handbook, Refrigeration Systems and Applications, chapter 8, titled “Lubricants in Refrigeration Systems”, pages 8.1 through 8.21, herein incorporated by reference. Lubricants of the present invention may comprise those commonly known as “mineral oils” in the field of compression refrigeration lubrication. Mineral oils comprise paraffins (i.e. straight-chain and branched-carbon-chain, saturated hydrocarbons), naphthenes (i.e. cyclic or ring structure saturated hydrocarbons, which may be paraffins) and aromatics (i.e. unsaturated, cyclic hydrocarbons containing one or more rings characterized by alternating double bonds). Lubricants of the present invention further comprise those commonly known as “synthetic oils” in the field of compression refrigeration lubrication. Synthetic oils comprise alkylaryls (i.e., linear and branched alkyl alkylbenzenes), synthetic paraffins and naphthenes, silicones, and polyalphaolefins. Representative conventional lubricants of the present invention are the commercially available BVM 100 N (paraffinic mineral oil sold by BVA Oils), napthenic mineral oil commercially available under the trademark from Suniso® 3GS and Suniso® 5GS by Crompton Co., naphthenic mineral oil commercially available from Pennzoil under the trademark Sontex® 372LT, naphthenic mineral oil commercially available from Calumet Lubricants under the trademark Calumet® RO-30, linear alkylbenzenes commercially available from Shrieve Chemicals under the trademarks Zerol® 75, Zerol® 150 and Zerol® 500 and branched alkylbenzene, sold by Nippon Oil as HAB 22.

Lubricants of the present invention further comprise those which have been designed for use with hydrofluorocarbon refrigerants and are miscible with refrigerants of the present invention under compression refrigeration and air-conditioning apparatus' operating conditions. Such lubricants and their properties are discussed in “Synthetic Lubricants and High-Performance Fluids”, R. L. Shubkin, editor, Marcel Dekker, 1993. Such lubricants include, but are not limited to, polyol esters (POEs) such as Castrol® 100 (Castrol, United Kingdom), polyalkylene glycols (PAGs) such as RL-488A from Dow (Dow Chemical, Midland, Mich.), and polyvinyl ethers (PVEs) such as PVE-FVC68D.

In one particular embodiment, the foregoing refrigerant compositions are combined with a PAG lubricant or a POE lubricant for usage in an automotive A/C system having an internal combustion engine or an electric or hybrid electric drive train.

In the compositions of the present invention including a lubricant, the lubricant may be present in an amount of less than 80 weight percent of the total composition. The lubricant may further be present in an amount of less than 60 weight percent of the total composition. In other embodiments, the amount of lubricant may be between about 0.1 and 50 weight percent of the total composition. The lubricant may also be between about 0.1 and 20 weight percent of the total composition. The lubricant may also be between about 0.1 and 5 weight percent of the total composition.

In one aspect of the invention, the inventive refrigerant composition is used to introduce lubricant into the A/C system as well as or alternatively other additives, such as a) acid scavengers, b) performance enhancers, and c) flame suppressants. In one preferred embodiment, the present compositions comprise an acid scavenger.

Examples of the acid scavengers that may be included in the present compositions include, but are not limited, the stabilizers and/or the epoxide component of the stabilizers disclosed in U.S. Pat. No. 8,535,555 and the acid scavengers disclosed in International Application Publication No. WO 2020/222864, the disclosure of each of which is incorporated herein by reference in its entirety.

In some embodiments, an acid scavenger may comprise one or more epoxides, one or more amines and/or one or more hindered amines, such as, for example but not limited to, epoxybutane.

In some embodiments, an acid scavenger may comprise a siloxane, an activated aromatic compound, or a combination of both. Serrano et al (paragraph 38 of US 2011/0272624 A1), which is hereby incorporated by reference, discloses that the siloxane may be any molecule having a siloxyfunctionality. The siloxane may include an alkyl siloxane, an aryl siloxane, or a siloxane containing mixtures of aryl and alkyl substituents. For example, the siloxane may be an alkyl siloxane, including a dialkylsiloxane or a polydialkylsiloxane. Preferred siloxanes include an oxygen atom bonded to two silicon atoms, i.e., a group having the structure: SiOSi. For example, the siloxane may be a siloxane of Formula IV: R1[Si(R2R3)4O]nSi(R2R3)R4, where n is 1 or more. Siloxanes of Formula IV have n that is preferably 2 or more, more preferably 3 or more, (e.g., about 4 or more). Siloxanes of formula IV have n that is preferably about 30 or less, more preferably about 12 or less, and most preferably about 7 or less. Preferably the R4 group is an aryl group or an alkyl group. Preferably the R2 groups are aryl groups or alkylgroups or mixtures thereof. Preferably the R3 groups are aryl groups or alkyl groups or mixtures thereof. Preferably the R4 group is an aryl group or an alkyl group. Preferably R1, R2, R3, R4, or any combination thereof are not hydrogen. The R2 groups in a molecule may be the same or different. Preferably the R2 groups in a molecule are the same. The R2 groups in a molecule may be the same or different from the R3 groups. Preferably, the R2 groups and R3 groups in a molecule are the same. Preferred siloxanes include siloxanes of Formula IV, wherein R1, R2, R3, R4, R5, or any combination thereof is a methyl, ethyl, propyl, or butyl group, or any combination thereof. Exemplary siloxanes that may be used include hexamethyldisiloxane, polydimethylsiloxane, polymethylphenylsiloxane, dodecamethylpentasiloxane, decamethylcyclo-pentasiloxane, decamethyltetrasiloxane, octamethyltrisiloxane, or any combination thereof.

Incorporated by previous reference from Serrano et al paragraph notes that in one aspect of the invention, the siloxane is an alkylsiloxane containing from about 1 to about 12 carbon atoms, such as hexamethyldisiloxane. The siloxane may also be a polymer such as polydialkylsiloxane, Where the alkyl group is a methyl, ethyl, propyl, butyl, or any combination thereof. Suitable polydialkylsiloxanes have a molecular weight from about 100 to about 10,000. Highly preferred siloxanes include hexamethyldisiloxane, polydimethylsiloxane, and combinations thereof. The siloxane may consist essentially of polydimethylsiloxane, hexamethyldisoloxane, or a combination thereof.

The activated aromatic compound may be any aromatic molecule activated towards a Friedel-Crafts addition reaction, or mixtures thereof. An aromatic molecule activated towards a Friedel-Crafts addition reaction is defined to be any aromatic molecule capable of an addition reaction with mineral acids. Especially aromatic molecules capable of addition reactions with mineral acids either in the application environment (AC system) or during the ASHRAE 97:2007 “Sealed Glass Tube Method to Test the Chemical Stability of Materials for Use within Refrigerant Systems” thermal stability test. Such molecules or compounds are typically activated by substitution of a hydrogen atoms of the aromatic ring with one of the following groups: NH2, NHR, NRz, ADH, AD, NHCOCH3, NHCOR, 4OCH3, OR, CH3, 4C2H5, R, or C6H5, where R is a hydrocarbon (preferably a hydrocarbon containing from about 1 to about 100 carbon atoms). The activated aromatic molecule may be an alcohol, or an ether, where the oxygen atom (i.e., the oxygen atom of the alcohol or ether group) is bonded directly to an aromatic group. The activated aromatic molecule may be an amine Where the nitrogen atom (i.e., the nitrogen atom of the amine group) is bonded directly to an aromatic group. By way of example, the activated aromatic molecule may have the formula ArXRn, Where X is O (i.e., oxygen) or N (i.e., nitrogen); n: 1 When X: O; n: 2 When x: N; Ar is an aromatic group (i.e., group, C6H5); R may be H or a carbon containing group; and When n: 2, the R groups may be the same or different. For example, R may be H (i.e., hydrogen), Ar, an alkyl group, or any combination thereof, Exemplary activated aromatic molecules that may be employed in a refrigerant composition according to the teachings herein include diphenyl oxide (i.e., diphenyl ether), methyl phenyl ether (e.g., anisole), ethyl phenyl ether, butyl phenyl ether or any combination thereof. One highly preferred aromatic molecule activated to Wards a Friedel-Crafts addition reaction is diphenyl oxide.

Incorporated by previous reference from Serrano et al. The acid scavenger (e.g., the activated aromatic compound, the siloxane, or both) may be present in any concentration that results in a relatively low total acid number, a relatively low total halides concentration, a relatively low total organic acid concentration, or any combination thereof.

Preferably the acid scavenger is present at a concentration greater than about 0.0050 wt %, more preferably greater than about 0.05 wt % and even more preferably greater than about 0.1 wt % (e.g., greater than about 0.5 wt %) based on the total weight of the refrigerant composition. The acid scavenger preferably is present in a concentration less than about 5 wt %, less than about 4 wt %, less than about 3 wt %, more preferably less than about 2.5 wt % and most preferably greater than about 2 wt % (e.g. less than about 1.8 wt %) based on the total weight of the refrigerant composition.

Additional examples of acid scavengers which may be included in the refrigerant composition and preferably are excluded from the refrigerant composition include those described by Kaneko (U.S. patent application Ser. No. 11/575,256, published as U.S. Patent Publication 2007/0290164, paragraph 42, expressly incorporated herein by reference), such as one or more of: phenyl glycidyl ethers, alkyl glycidyl ethers, alkyleneglycolglycidylethers, cyclohexeneoxides, otolenoxides, or epoxy compounds such as epoxidized soybean oil, and those described by Singh et al. (U.S. patent application Ser. No. 11/250,219, published as 20060116310, paragraphs 34-42, expressly incorporated herein by reference).

Preferred additives include those described in U.S. Pat. Nos. 5,152,926; 4,755,316, which are hereby incorporated by reference. In particular, the preferred extreme pressure additives include mixtures of (A) tolyltriazole or substituted derivatives thereof, (B) an amine (e.g., Jeffamine M-600) and (C) a third component which is (i) an ethoxylated phosphate ester (e.g. Antara LP-700 type), or (ii) a phosphate alcohol (e.g. ZELEC 3337 type), or (iii) a Zinc dialkyldithiophosphate (e.g. Lubrizol 5139, 5604, 5178, or 5186 type), or (iv) a mercaptobenzothiazole, or (v) a 2,5-dimercapto-1,3,4-triadiaZole derivative (e.g., Curvan 826) or a mixture thereof. Additional examples of additives which may be used are given in U.S. Pat. No. 5,976,399 (Schnur, 5:12-6:51, hereby incorporated by reference).

Acid number is measured according to ASTM D664-01 in units of mg KOH/g. The total halides concentration, the fluorine ion concentration, and the total organic acid concentration is measured by ion chromatography. Chemical stability of the refrigerant system is measured according to ASHRAE 97:2007 (RA 2017) “Sealed Glass Tube Method to Test the Chemical Stability of Materials for Use within Refrigerant Systems”. The viscosity of the lubricant is tested at 40° C. according to ASTM D-7042.

Mouli et al. (WO 2008/027595 and WO 2009/042847) teach the use of alkyl silanes as a stabilizer in refrigerant compositions containing fluoroolefins. Phosphates, phosphites, epoxides, and phenolic additives also have been employed in certain refrigerant compositions. These are described for example by Kaneko (U.S. patent application Ser. No. 11/575,256, published as U.S. Publication 2007/0290164) and Singh et al. (U.S. patent application Ser. No. 11/250,219, published as U.S. Publication 2006/0116310). All of these aforementioned applications are expressly incorporated herein by reference.

Preferred flame suppressants include the flame retardants described in patent application “Refrigerant compositions containing fluorine substituted olefins CA 2557873 A1” and incorporated by reference, as well as fluorinated products such as HFC-125 and/or Krytox® lubricants, also incorporated by reference and described in patent application “Refrigerant compositions comprising fluoroolefins and uses thereof WO2009018117A1.”

In one embodiment, compositions of the present invention include a composition comprising HFO-1234ze(Z) and at least one acid scavenger. In particular, in some embodiments, any of the HFO-1234ze(Z) compositions disclosed herein may include at least one acid scavenger.

Additionally, the present compositions may further comprise at least one tracer compound or mixture of tracer compounds. Tracers may be used to identify the process by which a refrigerant, or refrigerant mixture is produced. The tracer compounds may be specific to the manner of production or may be added as a single tracer or mixture of tracers in particular amounts in order to detect dilution, adulteration, contamination, or other unauthorized practices.

The tracer may be a single compound or two or more tracer compounds from the same class of compounds or from different classes of compounds. In some embodiments, the tracer is present in the compositions at a total concentration of about 1 part per million by weight (ppm) to about 5000 ppm, based on the weight of the total composition. In other embodiments, the tracer is present at a total concentration of about 1 ppm to about 1000 ppm. In other embodiments, the tracer is present at a total concentration of about 2 ppm to about 500 ppm. Alternatively, the tracer is present at a total concentration of about 10 ppm to about 300 ppm.

The tracer compound or compounds in amounts up to 100 ppm, 200 ppm, 300, ppm, 400 ppm, 500 ppm, 600 ppm, 700 ppm, 800 ppm and 900 ppm may be selected from hydrofluorocarbons, hydrofluoroolefins, hydrochlorocarbons, hydrochloroolefins, hydrochlorofluorocarbons, hydrochlorofluoroolefins, hydrochlorocarbons, hydrochloroolefins, chlorofluorocarbons, chlorofluoroolefins, hydrocarbons, perfluorocarbons, perfluoroolefins, and combinations thereof. In particular, the tracers may include, but are not limited to compounds selected from HFC-23 (trifluoromethane), HCFC-31 (chlorofluoromethane), HFC-41 (fluoromethane), HFC-161 (fluoroethane), HFC-152a (1,1-difluoromethane), HFC-143a (1,1,1-trifluoroethane), HFC-227ca (1,1,1,2,2,3,3-heptafluoropropane), HFC-227ea (1,1,1,2,3,3,3-heptafluoropropane), HFC-236fa (1,1,1,3,3,3-hexafluoropropane), HFC-236cb (1,1,1,2,2,3-hexafluoropropane), HFC-236ea (1,1,1,2,3,3-hexafluoropropane), HFC-245cb (1,1,1,2,2-pentafluoropropane), HFC-245fa (1,1,1,3,3-pentafluoropropane) HFC-245eb (1,1,1,2,3-pentafluoropropane), HFC-254eb (1,1,1,2-tetrafluoropropane), HFC-263fb (1,1,1-trifluoropropane), HFC-272ca (2,2-difluoropropane), HFC-281ea (2-fluoropropane), HFC-281fa (1-fluoropropane), HFC-329p (1,1,1,2,2,3,3,4,4-nonafluorobutane), HFC-329mmz (2-trifluoromethyl-1,1,1,3,3,3-hexafluoropropane), HFC-338mf (1,1,1,2,2,4,4,4-octafluorobutane), HFC-338pcc (1,1,2,2,3,3,4,4-octafluorobutane), CFC-12 (dichlorodifluoromethane), CFC-11 (trichlorofluoromethane), CFC-114 (1,2-dichloro-1,1,2,2-tetrafluoroethane), CFC-114a (2,2-dichloro-1,1,1,2-tetrafluoroethane), CFC-115 (chloropentafluoroethane), HCFC-22 (chlorodifluoromethane), HCFC-123 (2,2-dichloro-1,1,1-trifluoroethane), HCFC-124 (2-chloro-1,1,1,2-tetrafluoroethane), HCFC-124a (1-chloro-1,1,2,2-tetrafluoroethane), HCFC-141b (1,1-dichloro-1-fluoroethane), HCFC-142b (1-chloro-1,1-difluoroethane), HCFC-151a (1-chloro-1-fluoroethane), HCFC-244bb (2-chloro-1,1,1,2-tetrafluoropropane), HCC-40 (chloromethane), HFO-1141 (fluoroethylene), HCO-1130 (1,2-dichloroethylene, E- and/or Z-isomer), HCO-1130a (1,1-dichloroethylene), HCFO-1131 (1-chloro-2-fluoroethylene, E- and/or Z-isomer), HCFO-1131a (1-chloro-1-fluoroethylene), HCFO-1122 (2-chloro-1,1-difluoroethylene), HFO-1123 (trifluoroethylene), HFO-1234ye (1,2,3,3-tetrafluoropropene), HFO-1243zf (3,3,3-trifluoropropene), HFO-1225yeZ (1,2,3,3,3-pentafluoropropene), HFO-1225zc (1,1,3,3,3-pentafluoropropene), PFC-116 (hexafluoroethane), PFC-C216 (hexafluorocyclopropane), PFC-218 (octafluoropropane), PFC-C318 (octafluorocyclebutane), PFC-1216 (hexafluoropropene), PFC-31-10mc (decafluorobutane), PFC-31-10my (2-trifluoromethyl-1,1,1,2,3,3,3-heptafluoropropane), 2-chloro-1,1,2-trifluoroethylene (CFO-1113), 1,1,1,3,3-pentafluorobutane (HFC-365mfc), 1,1,1,2,3,4,4,5,5,5-decafluoropentane (HFC-43-10mee), 1,1,1,2,2,3,4,5,5,6,6,7,7,7-tetradecafluoroheptane, hexafluorobutadiene, 3,3,3-trifluoropropyne, deuterated hydrocarbons, deuterated hydrofluorocarbons, perfluorocarbons, fluoroethers, and mixtures thereof.

In some embodiments, the tracer is a blend containing two or more hydrofluorocarbons, or one hydrofluorocarbon in combination with one or more perfluorocarbons. In other embodiments, the tracer is a blend of at least one CFC and at least one HCFC, HFC, or PFC. In one embodiment, compositions of the present invention may comprise HFO-1234ze(Z), HFO-1336mzz(Z), HFO-1336mzz(E), HCFO-1224 yd(E), HCFO-1224 yd(Z), HFO-1327mz(Z), HFO-1327mz(E), one or more hydrofluoroethers, and one or more hydrofluorocarbons.

In some embodiments, where the composition comprises HFO-1234ze(Z) and HFO-1234yf, and optionally additional compounds or components, the composition further preferably comprises an olefin polymerization inhibitor. Examples of the inhibitor that may be included in the present compositions include, but are not limited, the inhibitors disclosed in International Application Publication No. WO 2020/222864, the disclosure of which is incorporated herein by reference in its entirety.

### Processes of Making

In one embodiment, the present invention relates to methods of producing a fluoropropene of formula CF₃CH═CHF, particularly the Z isomer. The method comprises contacting a starting material in the gas phase with a catalyst, optionally in the presence of one of an oxygen containing gas or a fluorinating agent such as hydrogen fluoride, to form a reaction mixture comprising Z-1,3,3,3-tetrafluoropropene, E-1,3,3,3-tetrafluoropropene, and optionally additional constituents. In one embodiment, the starting material is selected from HFC-245fa, HCC-240fa and HCFO-1233zd.

In one embodiment, HFO-1234ze may be prepared by dehydrofluorination of 1,1,1,3,3-pentafluoropropane (HFC-245fa or CF₃CH₂CHF₂) using a strong base in aqueous or alcoholic solution or by means of chromium-containing catalyst in the presence of oxygen at elevated temperature. Certain dehydrofluorinations are known in the art and are preferably conducted in the vapor phase. The dehydrofluorination reaction may take place in the vapor phase in the presence or absence of catalyst, and also in the liquid phase by reaction with a caustic composition, such as NaOH or KOH. These reactions are described in more detail in U.S. Patent Publication No. 2006/0106263, incorporated herein by reference.

According to this process embodiment, the instant invention relates to feeding an amount of HFC-245fa to a dehydrofluorination reactor containing a catalyst in the presence of an oxygen containing gas, such as air, at a predetermined rate and a predetermined temperature. In one embodiment, the oxygen containing gas is present in an amount from greater than 0 ppm to less than 10 mol % of the total feed. In one embodiment, the reactor is preferably a fixed bed reactor. The HFC-245fa converted by dehydrofluorination produces Z-HFO-1234ze and E-HFO-1234ze, among other compounds. The process thus further comprises separating Z-HFO-1234ze from the reaction product, and optionally purifying the Z-HFO-1234ze, such as by adsorption or another conventional purification method known in the art.

According to this process, both E-HFO-1234ze and Z-HFO-1234ze are produced from the HFC-245a. The conversion is between about 10% to about 100%. In some embodiments, unreacted HFC-245a is recycled back to the feed of the reactor, optionally with a small amount of Z-HFO-1234ze. E-HFO-1234ze is separated from the reaction mixture or reaction product and isomerized to make Z-HFO-1234ze. The E-HFO-1234ze may be optionally purified before the isomerization reaction.

In one embodiment, the dehydrofluorination reaction may be carried out at a temperature of between about 200° C. to about 400° C., or between about 250° C. to about 375° C., or about 250° C. to about 350° C., and in some cases at a temperature of about 370° C.

In one embodiment, the contact time is typically from about 10 to about 80 seconds, and more preferably from about 30 to about 60 seconds, and most preferably from about 45 to about 50 seconds.

The reaction pressure can be subatmospheric, atmospheric, or superatmospheric. In one embodiment, the reaction is conducted at a pressure of from 14 psig to about 100 psig. In another embodiment, the reaction is conducted at a pressure of from 14 psig to about 60 psig. In yet another embodiment, the reaction is conducted at a pressure of from 40 psig to about 85 psig. In yet another embodiment, the reaction is conducted at a pressure of from 50 psig to 75 psig. In general, increasing the pressure in the reactor above atmospheric pressure will act to increase the contact time of the reactants in the process. Longer contact times will necessarily increase the degree of conversion in a process, without having to increase temperature.

Depending on the temperature of the reactor and the contact time, the product mixture from the reactor will contain varying amounts of unreacted HFC-245fa and other constituents. More particularly, depending on the temperature of the reactor and the contact time, the reactor effluent of this process embodiment using HFC-245fa as the feed may include one or more of HFO-1141, HFC-143a, HFC-152a, trifluoropropyne, HFO-1234yf, E-HFO-1234ze, Z-HFO-1234ze, HFC-245fa, E-HCFO-1233zd and Z-HCFO-1233zd.

In one embodiment, the reactor feed is preheated in a vaporizer to a temperature of from about 30° C. to about 100° C. In another embodiment, the reactor feed is preheated in a vaporizer to a temperature of from about 30° C. to about 80° C.

The catalyst can be readily regenerated by any means known in the art if they become deactivated. For example, an oxygen containing gas may be supplied for regeneration of the catalyst.

Also disclosed herein, in one embodiment, is a composition comprising the compound Z-HFO-1234ze produced by the dehydrofluorination reaction of this first process embodiment. In certain embodiments, the reaction mixture produced by the dehydrofluorination reaction of this first process embodiment comprises a composition comprising HFO-1234ze(Z) and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; or at least two additional compounds or at least three additional compounds or more.

In a second process embodiment, HFO-1234ze may be prepared from 1,1,1,3,3-pentachloropropane (HCC-240fa or CCl3CH2CHCl2). According to this second process embodiment, the present invention comprises fluorination of HCC-240fa by reaction with a fluorinating agent, such as hydrogen fluoride, in a reactor, preferably in the vapor phase, in the presence of a fluorinated catalyst, to make a reaction mixture comprising Z-HFO-1234ze. The Z-HFO-1234ze is separated from other compounds of the reaction mixture, and optionally purified such as by adsorption or another conventional purification method known in the art.

The fluorination agent is preferably anhydrous or substantially anhydrous. By “substantially anhydrous,” we mean that the fluorination agent contains less than about 0.05 wt. % water, and preferably contains less than about 0.02 wt. % water.

In some embodiments, the fluorinating agent is selected from hydrogen fluoride, antimony trifluoride, antimony tetrafluoride, antimony pentafluoride, antimony trichloride/hydrogen fluoride, antimony tetrachloride/hydrogen fluoride, or any mixture thereof. In some embodiments, the fluorinating agent is hydrogen fluoride (HF). In some embodiments, the HF is preferably anhydrous or substantially anhydrous. By “substantially anhydrous,” we mean that the HF contains less than about 0.05 wt. % water, and preferably contains less than about 0.02 wt. % water.

The fluorinated catalyst can be readily regenerated by any means known in the art if they become deactivated. For example, an oxygen containing gas may be supplied for regeneration of the catalyst.

In one embodiment, the fluorination reaction may be performed by introducing the HCC-240a starting material and the fluorinating agent into a reaction vessel or zone, and then heating the mixture with agitation.

The reactor is preferably preheated to a fluorination reaction temperature while anhydrous or substantially anhydrous HF is fed to the reactor. The HCC-240fa and HF may be fed to the reactor at any convenient temperature and pressure. In a one embodiment, either or both of the HCC-240fa and HF are pre-vaporized or preheated to a temperature of from about 30° C. to about 300° C., preferably about 150° C., prior to entering the reactor. In another embodiment, the HCC-240fa and HF are vaporized in the reactor.

In some embodiments, the HF and HCC-240fa feeds may be adjusted to the desired mole ratio. The HF to HCC-240fa mole ratio preferably ranges from about 3:1 to about 100:1; more preferably from about 4:1 to about 50:1 and most preferably from about 5:1 to about 20:1. In a preferred embodiment, the HF to HCC-240fa mole ratio is 20:1.

In other embodiments, the reactor is a fixed bed reactor.

In one embodiment, the contact time for the fluorination reaction may be from about 1 to about 90 seconds, preferably about 3 to about 60 seconds, and more preferably from about 5 to about 30 seconds.

After introduction of the feed reactants, the temperature of the reactor is increased. The fluorination reaction is conducted at a temperature ranging from about 80° C. to about 400° C., more preferably from about 100° C. to about 375° C., and most preferably from about 200° C. to about 350° C.

The reaction pressure can be subatmospheric, atmospheric, or superatmospheric. In one embodiment, the reaction is conducted at a pressure of from 14 psig to about 100 psig. In another embodiment, the reaction is conducted at a pressure of from 14 psig to about 60 psig. In yet another embodiment, the reaction is conducted at a pressure of from 40 psig to about 85 psig. In yet another embodiment, the reaction is conducted at a pressure of from 50 psig to 75 psig. In general, increasing the pressure in the reactor above atmospheric pressure will act to increase the contact time of the reactants in the process. Longer contact times will necessarily increase the degree of conversion in a process, without having to increase temperature.

The HFC-240fa converted by the reaction produces Z-HFO-1234ze, HFC-245fa and E-HFO-1234ze, among other compounds. The conversion to E-HFO-1234ze and Z-HFO-1234ze is between about 10% to about 100%. Depending on the temperature of the reactor and the contact time, the product mixture from the reactor will contain varying amounts of unreacted HFC-240fa and other constituents. More particularly, depending on the temperature of the reactor and the contact time, the reactor effluent may include one or more of E-HFO-1234ze, Z-HFO-1234ze, HFC-245fa, HFO-1233xf, E-HCFO-1233zd and Z-HCFO-1233zd, HCFC-244fa, HFC-243fa and HFC-243fb. The process thus further comprises separating the desired Z-HFO-1234ze from the reaction product.

In some embodiments, unreacted HFC-240fa, along with R-1233zd, R-244fa and/or R-243fa are recycled back to the feed of the reactor, optionally with a small amount of Z-HFO-1234ze. E-HFO-1234ze is separated from the reaction mixture or reaction product and isomerized to make Z-HFO-1234ze. The E-HFO-1234ze may be optionally purified before the isomerization reaction.

Also disclosed herein, in one embodiment, is a composition comprising the compound Z-HFO-1234ze produced by the fluorination reaction of this second process embodiment. In certain embodiments, the reaction mixture produced by the fluorination reaction of this second process embodiment comprises a composition comprising HFO-1234ze(Z) and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; or at least two additional compounds or at least three additional compounds or more.

In a third process embodiment, HFO-1234ze may be prepared from 1-chloro-3,3,3-trifluoropropene (HCFO-1233zd or CF3CH═CHCl). According to this third process embodiment, the present invention comprises fluorination of HCFO-1233zd by reaction with a fluorinating agent, such as HF, in a reactor, preferably in the vapor phase, in the presence of a fluorinated catalyst, to make a reaction mixture comprising Z-HFO-1234ze. The Z-HFO-1234ze is separated from other compounds of the reaction mixture, and optionally purified such as by adsorption or another conventional purification method known in the art.

The fluorination agent is preferably anhydrous or substantially anhydrous. By “substantially anhydrous,” we mean that the fluorination agent contains less than about 0.05 wt. % water, and preferably contains less than about 0.02 wt. % water.

In some embodiments, the fluorinating agent is selected from hydrogen fluoride, antimony trifluoride, antimony tetrafluoride, antimony pentafluoride, antimony trichloride/hydrogen fluoride, antimony tetrachloride/hydrogen fluoride, or any mixture thereof. In some embodiments, the fluorinating agent is hydrogen fluoride (HF). In some embodiments, the HF is preferably anhydrous or substantially anhydrous. By “substantially anhydrous,” we mean that the HF contains less than about 0.05 wt. % water, and preferably contains less than about 0.02 wt. % water.

The fluorinated catalyst can be readily regenerated by any means known in the art if they become deactivated. One suitable method of regenerating the catalyst involves, for example, supply oxygen containing gas to the catalyst system.

In one embodiment, the fluorination reaction may be performed by introducing the HCFO-1233zd starting material and the fluorinating agent into a reaction vessel or zone, and then heating the mixture with agitation.

The reactor is preferably preheated to a fluorination reaction temperature while anhydrous or substantially anhydrous HF is fed to the reactor. The HCFO-1233zd and HF may be fed to the reactor at any convenient temperature and pressure. In a one embodiment, either or both of the HCFO-1233zd and HF are pre-vaporized or preheated to a temperature of from about 30° C. to about 150° C., preferably about 80° C., prior to entering the reactor. In another embodiment, the HCFO-1233zd and HF are vaporized in the reactor.

In some embodiments, the HF and HCFO-1233zd feeds may be adjusted to the desired mole ratio. The HF to HCFO-1233zd mole ratio preferably ranges from about 3:1 to about 100:1; more preferably from about 4:1 to about 50:1 and most preferably from about 5:1 to about 20:1. In a preferred embodiment, the HF to HCFO-1233zd mole ratio is 20:1.

In other embodiments, the reactor is a fixed bed reactor.

In one embodiment, the contact time for the fluorination reaction may be from about 1 to about 90 seconds, preferably about 3 to about 60 seconds, and more preferably from about 5 to about 30 seconds.

After introduction of the feed reactants, the temperature of the reactor is increased. The fluorination reaction is conducted at a temperature ranging from about 80° C. to about 400° C., more preferably from about 100° C. to about 375° C., and most preferably from about 300° C. to about 350° C.

The reaction pressure can be subatmospheric, atmospheric, or superatmospheric. In one embodiment, the reaction is conducted at a pressure of from 14 psig to about 100 psig. In another embodiment, the reaction is conducted at a pressure of from 14 psig to about 60 psig. In yet another embodiment, the reaction is conducted at a pressure of from 40 psig to about 85 psig. In yet another embodiment, the reaction is conducted at a pressure of from 50 psig to 75 psig. In general, increasing the pressure in the reactor above atmospheric pressure will act to increase the contact time of the reactants in the process. Longer contact times will necessarily increase the degree of conversion in a process, without having to increase temperature.

The HCFO-1233zd converted by the reaction produces Z-HFO-1234ze, HCFO-1233zd (E and Z isomers) and E-HFO-1234ze, among other compounds. The conversion to Z-HFO-1234ze and E-HFO-1234ze is preferably from about 10% to about 100%. Depending on the temperature of the reactor and the contact time, the product mixture from the reactor will contain varying amounts of unreacted E- and Z-HCFO-1233zd, as well as other constituents. More particularly, depending on the temperature of the reactor and the contact time, the reactor effluent may include one or more of E-HFO-1234ze, Z-HFO-1234ze, E-HCFO-1233zd, Z-HCFO-1233zd, HFO-1234yf, HFC-236fa, HFC-245fa, HFO-1233xf and HFC-243fa. The process thus further comprises separating the desired Z-HFO-1234ze from the reaction product.

In some embodiments, unreacted HCFO-1233zd is recycled back to the feed of the reactor, optionally with a small amount of Z-HFO-1234ze. E-HFO-1234ze is separated from the reaction mixture or reaction product and isomerized to make Z-HFO-1234ze. The E-HFO-1234ze may be optionally purified before the isomerization reaction.

Also disclosed herein, in one embodiment, is a composition comprising the compound Z-HFO-1234ze produced by the fluorination reaction of this third process embodiment. In certain embodiments, the reaction mixture produced by the fluorination reaction of this second process embodiment comprises a composition comprising HFO-1234ze(Z) and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; or at least two additional compounds or at least three additional compounds or more.

In some embodiments, for any of the processes described herein, an inert diluent gas is used as a carrier gas for the hydrochlorofluoropropane. In one embodiment, the carrier gas is selected from nitrogen, argon, helium, or carbon dioxide.

The reactor or vessel, distillation columns, feed lines, effluent lines and any other associated units utilized in carrying out any of the process embodiments disclosed herein should be constructed from materials which are resistant to the corrosive effects of hydrogen fluoride, such as nickel and its alloys, including Hastelloy, Monel, and Inconel, or vessels lined with fluoropolymers. These may be a single tube, or multiple tubes packed with an appropriate catalyst depending on the reaction to be carried out.

Useful catalysts for the processes of any of the process embodiments disclosed herein include chromium-based catalysts such as fluorinated chromium oxide, and aluminum-based catalysts such as fluorinated alumina oxide. The catalyst may either be unsupported, or supported on a support such as activated carbon, graphite, fluoride graphite, or alumina fluoride. The catalyst may either be used alone, or in the presence of a co-catalyst selected from nickel, cobalt, manganese or zinc salt. In one embodiment, a chromium catalyst is high surface area chromium oxide, or chromium/nickel on alumina fluoride (Cr/Ni/AlF₃), the preparation of which is reported in European Patent EP486,333. In another embodiment, the catalyst is fluorinated Guignet's green catalyst. Additional suitable catalysts include, but are not limited to, JM 62-2 (chrome catalyst available from Johnson Matthey), LV (chrome catalyst available from Chemours), JM-62-3 (chrome catalyst available from Johnson Matthey), and Newport Chrome (chrome catalyst available from Chemours). The chromium catalysts are preferably activated before use, typically by a procedure whereby the catalyst is heated to from 350° C. to 400° C. under a flow of nitrogen for a period of time, after which the catalyst is heated under a flow of HF and nitrogen or air for an additional period of time.

In one embodiment, the Guignet's Green of the fluoride-activated Guignet's Green catalyst used in the present invention is made by reacting (fusing) boric acid with alkali metal dichromate at 500° C. to 800° C., followed by hydrolysis of the reaction product, whereby said Guignet's Green contains boron, alkali metal, and water of hydration. The usual alkali metal dichromates are the Na and/or K dichromates. The reaction is typically followed by the steps of cooling the reaction product in air, crushing this solid to produce a powder, followed by hydrolysis, filtering, drying, milling and screening. The Guignet's Green is bluish green, but is known primarily as a green pigment, whereby the pigment is commonly referred to as Guignet's Green. When used as a catalyst, it is also referred to as Guignet's Green as disclosed in U.S. Pat. No. 3,413,363. In U.S. Pat. No. 6,034,289, Cr₂O₃ catalysts are disclosed as preferably being in the alpha form, and Guignet's Green is also disclosed as a commercially available green pigment having the composition: Cr₂O₃ 79-83%, H₂O 16-18%, B₂O₅ 1.5 to 2.7% (sentence bridging cols. 2 and 3) that can be converted to the alpha form (col. 3, l. 3). U.S. Pat. No. 7,985,884 acknowledges the presence of alkali metal in the Guignet's Green in the composition of Guignet's Green disclosed in Example 1:54.5% Cr, 1.43% B, 3,400 ppm Na, and 120 ppm K.

The physical shape of the catalyst is not critical and may, for example, include pellets, extrudates, powders, or granules. The fluoride activation of the catalyst is preferably carried out on the final shape of the catalyst.

For any of the process embodiments disclosed herein, the desired Z-HFO-1234ze may be purified by a conventional method for purifying reaction products and separated from the reaction mixture by methods known in the art (e.g., distillation). The E-HFO-1234ze may also be separated and recovered from the reaction mixture by known methods and further treated, as described below, for isomerization to Z-HFO-1234ze. Any unreacted feed materials may recycled back to the reactor with additional material for further production of the reaction mixture. Further, any excessive amount of hydrogen fluoride present may be removed by scrubbing, distillation, and the like.

In some embodiment, as briefly noted above, the separated and recovered E-HFO-1234ze, produced by any of the process embodiments disclosed herein, is isomerized to Z-HFO-1234ze. The isomerization step or method comprises reacting the E-HFO-1234ze, preferably in the vapor phase, with at least one fluorinated catalyst, optionally in the presence of an oxygen containing gas. The E-HFO-1234ze may optionally be purified before isomerization.

In one embodiment, the contacting for the isomerization reaction occurs at a reaction temperature from about 50° C. to about 450° C., preferably from about 50° C. to about 400° C., and more preferably 50° C. to about 375° C., to isomerize at least a portion the E-HFO-1234ze into Z-HFO-1234ze. The contact time is typically from about 2 to about 90 seconds, or from about 10 to about 70 seconds.

In some embodiments, a catalyst suitable for use in the isomerization reaction scheme includes a vapor phase chromium oxide (Cr₂O₃) or aluminum oxide (Al₂O₃) catalyst. In one embodiment, the isomerization catalyst includes chromium oxide supported on aluminum oxide. In one embodiment, the isomerization catalyst includes zinc doped chromium oxide. Suitable isomerization catalysts include, but are not limited to, chromium oxide, fluorinated chromium oxide, oxyfluorides of chrome, chromium halide, alumina, aluminum fluoride, fluorided alumina, metal compounds on aluminum fluoride, metal compounds on fluorided alumina; oxides, fluorides, and oxyfluorides of magnesium, zinc and mixtures of magnesium and zinc and/or aluminum; lanthanum oxide and fluorided lanthanum oxide; carbon, acid-washed carbon, activated carbon, three dimensional matrix carbonaceous materials; and metal compounds supported on carbon. The metal compounds are oxides, fluorides, and oxyfluorides of at least one metal selected from the group consisting of sodium, potassium, rubidium, cesium, yttrium, lanthanum, cerium, praseodymium, neodymium, samarium, chromium, iron, cobalt, rhodium, nickel, copper, zinc, and mixtures thereof. The catalyst is contacted for a time sufficient to affect the desired isomerization.

The reaction pressure used in the isomerization reaction can be sub-atmospheric, atmospheric or super-atmospheric. In one embodiment, the reaction pressure for the isomerization reaction is about 10 to about 150 psig.

In some embodiments, the overall conversion of E-HFO-1234ze into Z-HFO-1234ze may be about 2% to about 70%.

Additionally, the compositions of the present invention may be prepared from recycled or reclaimed refrigerant. One or more of the components may be recycled or reclaimed by means of removing contaminants, such as air, water, or residue, which may include lubricant or particulate residue from system components. The means of removing the contaminants may vary widely, but can include distillation, decantation, filtration, and/or drying by use of molecular sieves or other absorbents. Then the recycled or reclaimed component(s) may be combined with the other component(s), if needed, as described above.

### Utility and Systems

The compositions comprising HFO-1234ze(Z), discussed relative to the following utilities, systems and methods, include the single component composition disclosed herein and any of the blend compositions disclosed herein. For the sake of brevity, these compositions are collectively referred to in this discussion as the “HFO-1234ze(Z) composition” or “HFO-1234ze(Z) compositions”.

The HFO-1234ze(Z) compositions disclosed herein are useful as low global warming potential (GWP) heat transfer compositions, working fluids, aerosol propellants, foaming agents, blowing agents, solvents, cleaning agents, carrier fluids, displacement drying agents, buffing abrasion agents, polymerization media, expansion agents for polyolefins and polyurethane, gaseous dielectrics, extinguishing agents, and fire suppression agents in liquid or gaseous form. The disclosed compositions can act as a working fluid for heat transfer and refrigeration applications, particularly to carry heat from a heat source to a heat sink. Such heat transfer compositions may also be useful as a refrigerant in a cycle wherein the fluid undergoes a phase change; that is, from a liquid to a gas and back or vice versa.

In one embodiment, the HFO-1234ze(Z) compositions are useful in heat transfer systems. Examples of heat transfer systems include but are not limited to air conditioners, freezers, refrigerators, heat pumps, flooded evaporator heat pumps, direct expansion heat pumps, water chillers, flooded evaporator chillers, direct expansion chillers, walk-in coolers, mobile refrigerators, mobile air conditioning units and combinations thereof.

In one embodiment, the HFO-1234ze(Z) compositions are useful in mobile heat transfer systems, including refrigeration, air conditioning, or heat pump systems or apparatus. In another embodiment, the compositions are useful in stationary heat transfer systems, including refrigeration, air conditioning, chillers, or heat pump systems or apparatus. In a preferred embodiment, the HFO-1234ze(Z) compositions are useful in mobile or stationary high temperature heat pump systems or apparatus, particularly flooded evaporator heat pump systems and flooded evaporator chillers. In a preferred embodiment, the HFO-1234ze(Z) compositions are useful in flooded evaporators.

As used herein, mobile refrigeration apparatus, mobile air conditioning or mobile heating apparatus refers to any refrigeration, air conditioner, or heating apparatus incorporated into a transportation unit for the road, rail, sea or air. In addition, mobile refrigeration or air conditioner units, include those apparatus that are independent of any moving carrier and are known as “intermodal” systems. Such intermodal systems include “containers’ (combined sea/land transport) as well as “swap bodies” (combined road/rail transport).

As used herein, stationary heat transfer systems are systems that are fixed in place during operation. A stationary heat transfer system may be associated within or attached to buildings of any variety or may be stand-alone devices located out of doors, such as a soft drink vending machine. These stationary applications may be stationary air conditioning and heat pumps (including but not limited to chillers, high temperature heat pumps, including trans-critical heat pumps with condenser or supercritical heat rejection heat exchanger temperatures above 50° C., 70° C., 80° C., 100° C., 120° C., 140° C., 160° C., 180° C., or 200° C.), residential, commercial or industrial air conditioning systems, and including window, ductless, ducted, packaged terminal, chillers, and those exterior but connected to the building such as rooftop systems). In stationary refrigeration applications, the disclosed compositions may be useful in high temperature, medium temperature and/or low temperature refrigeration equipment including commercial, industrial or residential refrigerators and freezers, ice machines, self-contained coolers and freezers, flooded evaporator chillers, direct expansion chillers, walk-in and reach-in coolers and freezers, and combination systems. In some embodiments, the disclosed compositions may be used in supermarket refrigerator systems.

Therefore, in accordance with the present invention, the HFO-1234ze(Z) compositions as disclosed herein may be useful in methods for producing cooling, producing heating, and transferring heat.

The HFO-1234ze(Z) compositions disclosed herein may be useful as low global warming potential (GWP) replacements for currently used refrigerants, including but not limited to HCFO-1233zdE, HFO-1336mzzE and HFO-1336mzzZ, among others.

In many applications, some embodiments of the HFO-1234ze(Z) compositions are useful as refrigerants and provide at least comparable cooling performance (meaning cooling capacity and energy efficiency) as the refrigerant for which a replacement is being sought.

In another embodiment is provided a method for recharging a heat transfer system that contains a refrigerant to be replaced and a lubricant. The method comprises removing the refrigerant to be replaced from the heat transfer system while retaining a substantial portion of the lubricant in said system and introducing one of the HFO-1234ze(Z) compositions to the heat transfer system.

In one embodiment, a method for replacing a first refrigerant composition with a second refrigerant composition in a cooling or heating system is provided. The method comprises removing the first refrigerant composition from the cooling or heating system and charging second refrigerant composition to the cooling or heating system. In one embodiment, the first refrigerant is selected from any of R-1233zdE, R-1336mzzE and R-1336mzzZ, and the second refrigerant composition comprises any of the HFO-1234ze(Z) compositions disclosed herein.

In one embodiment, the first refrigerant is R-1233zd(E) and the second refrigerant is selected from:
a composition comprising HFO-1234ze(Z) and HFO-1336mzz(Z), and optionally one or more additional compounds selected from HFO-1336mzz(E), HFO-1327mz, HFO-1326mxz(Z), HFO-1326mxz(E), HFC-356mff, CHFC-346mdf, and HFC-263fb, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb;
a composition comprising HFO-1234ze(Z) and HFO-1336mzz(E), and optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb;
a composition comprising HFO-1234ze(Z), HFO-1336mzz(Z) and HFO-1336mzz(E), and optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and optionally one or more additional compounds selected from HFO-1336mzz(E), HFO-1327mz, HFO-1326mxz(Z), HFO-1326mxz(E), HFC-356mff, CHFC-346mdf, and HFC-263fb, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb;
a composition comprising HFO-1234ze(Z) and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb;
a composition comprising HFO-1234ze(Z), HFO-1336mzz(E) and HFC-245fa, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and/or optionally one or more additional compounds selected from CFC-141b, HCFC-235fa and HCFC-243fa;
a composition comprising HFO-1234ze(Z), HFO-1336mzz(E) and HFC-245fa, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and/or optionally one or more additional compounds such as butane;
a composition comprising HFO-1234ze(Z), HFO-1336mzz(E) and HFC-227ea, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and/or optionally one or more additional compounds selected from FC-1216 and HCFC-124;
a composition comprising HFO-1234ze(Z), HFO-1336mzz(E), HFO-1336mzz(Z), and HFC-134, and optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, optionally one or more additional compounds selected from HFO-1336mzz(E), HFO-1327mz, HFO-1326mxz(Z), HFO-1326mxz(E), HFC-356mff, CHFC-346mdf, and HFC-263fb, optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and/or optionally one or more additional compounds selected from HFC-134a, HCFC-124, CHFO-1122, HFC-143a, HFC-32, HFC-125, CFC-114 and CFC-114a;
a composition comprising HFO-1234ze(Z), HFO-1234ze(E), and HFC-134, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, optionally one or more additional compounds selected from HFC-134a, HFO-1225zc, HFO-1234yf, HFC-245cb, HFC-236fa, HFO-1225ye(E), HFO-1234zc, HFC-245fa, HCFC-124, HFO-1234ze(Z), CFC-114, trifluoropropyne, HFC-152a, HFO-1225ye(Z), HFO-1225ye(E), HCFO-1233xf, HFC-263fb, HFO-1243zf and HCFO-1233zd(E), and/or optionally one or more additional compounds selected from HFC-134a, HCFC-124, CHFO-1122, HFC-143a, HFC-32, HFC-125, CFC-114 and CFC-114a;
a composition comprising HFO-1234ze(Z), HFO-1234ze(E), HCFO-1233zdE and HFC-134, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, optionally one or more additional compounds selected from HFC-134a, HFO-1225zc, HFO-1234yf, HFC-245cb, HFC-236fa, HFO-1225ye(E), HFO-1234zc, HFC-245fa, HCFC-124, HFO-1234ze(Z), CFC-114, trifluoropropyne, HFC-152a, HFO-1225ye(Z), HFO-1225ye(E), HCFO-1233xf, HFC-263fb, HFO-1243zf and HCFO-1233zd(E), optionally one or more additional compounds selected from HFO-1234ze(Z), HCFO-1233zd(Z) and HFC-245fa, and/or optionally one or more additional compounds selected from HFC-134a, HCFC-124, CHFO-1122, HFC-143a, HFC-32, HFC-125, CFC-114 and CFC-114a;
a composition comprising HFO-1234ze(Z), HFO-1336mzz(Z), and HCO-1130(E), and optionally one or more additional compounds selected from HFO-1336mzz(E), HFO-1327mz, HFO-1326mxz(Z), HFO-1326mxz(E), HFC-356mff, CHFC-346mdf, and HFC-263fb, optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and/or optionally one or more additional compounds such as HCO-1130 (Z); and
a composition comprising HFO-1234ze(Z), HFO-1336mzz(Z) and HCFO-1224 yd(Z), and optionally one or more additional compounds selected from HFO-1336mzz(E), HFO-1327mz, HFO-1326mxz(Z), HFO-1326mxz(E), HFC-356mff, CHFC-346mdf, and HFC-263fb, optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, and/or optionally one or more additional compounds selected from HCFO-1224 yd(E), HFO-1234yf, HFC-254eb, HCFC-244bb, CFC-1215yb.

In one embodiment, the second refrigerant is either composition (i) or (ii).

In one embodiment, the first refrigerant is HFO-1336mzz(Z) and the second refrigerant is a composition comprising HFO-1234ze(Z), HFO-1336mzz(Z) and HFO-1336mzz(E), and optionally one or more additional compounds selected from HCFO-1233xf, HFO-1336ft, HCFC-133a, CO-1140, HCFO-1233zd(E), HFC-245fa, HFO-1327mz, HFC-347mef, HFO-1243zf, and optionally one or more additional compounds selected from HFO-1336mzz(E), HFO-1327mz, HFO-1326mxz(Z), HFO-1326mxz(E), HFC-356mff, CHFC-346mdf, and HFC-263fb, and optionally one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

In another embodiment, a heat exchange system containing any of the HFO-1234ze(Z) compositions is provided, wherein said system is selected from the group consisting of air conditioners, freezers, refrigerators, heat pumps, water chillers, walk-in coolers, heat pumps, mobile refrigerators, mobile air conditioning units, and systems having combinations thereof. Additionally, the HFO-1234ze(Z) compositions may be useful in secondary loop systems wherein these compositions serve as the primary refrigerant thus providing cooling to a secondary heat transfer fluid that thereby cools a remote location.

Vapor-compression refrigeration, air-conditioning, or heat pump systems include an evaporator, a compressor, a condenser, and an expansion device. A vapor-compression cycle re-uses refrigerant in multiple steps producing a cooling effect in one step and a heating effect in a different step. The cycle can be described simply as follows. Liquid refrigerant enters an evaporator through an expansion device, and the liquid refrigerant boils in the evaporator, by withdrawing heat from the environment, at a low temperature to form a gas and produce cooling. The low-pressure gas enters a compressor where the gas is compressed to raise its pressure and temperature. The higher-pressure (compressed) gaseous refrigerant then enters the condenser in which the refrigerant condenses and discharges its heat to the environment. The refrigerant returns to the expansion device through which the liquid expands from the higher-pressure level in the condenser to the low-pressure level in the evaporator, thus repeating the cycle.

In one embodiment, there is provided a heat transfer system containing any of the HFO-1234ze(Z) compositions. In another embodiment is disclosed a refrigeration, air-conditioning or heat pump apparatus, such as a flooded evaporator chiller or a flooded evaporator heat pump, containing any of the HFO-1234ze(Z) compositions. In another embodiment, is disclosed a stationary refrigeration or air-conditioning apparatus containing any of the HFO-1234ze(Z) compositions. In yet another embodiment is disclosed a mobile refrigeration or air conditioning apparatus containing a composition as disclosed herein.

In one embodiment, a method is provided for producing cooling comprising expanding any of the HFO-1234ze(Z) compositions in the vicinity of a body to be cooled, and thereafter compressing the composition.

In another embodiment, a method is provided for producing heating comprising compressing any of the HFO-1234ze(Z) compositions in the vicinity of a body to be heated, and thereafter expanding the composition.

The method for producing heating may further comprise passing a heat transfer medium through the condenser, whereby said condensation of working fluid heats the heat transfer medium and passing the heated heat transfer medium from the condenser to a body to be heated.

A body to be heated or cooled may be any space, object or fluid that may be heated such as water or air for space heating. In one embodiment, a body to be heated or cooled may be a room, building, or the passenger compartment of an automobile. Alternatively, in another embodiment, a body to be heated or cooled may be a second or the medium or heat transfer fluid, such as a chemical process stream.

In another embodiment, disclosed is a method of using the HFO-1234ze(Z) compositions as a heat transfer fluid composition. The method comprises transporting the working fluid from a heat source to a heat sink.

In another embodiment, the HFO-1234ze(Z) compositions of the present invention may be used to top-off a refrigerant charge in a heat transfer system. For instance, if a heat transfer system has diminished performance due to leakage of refrigerant, the compositions as disclosed herein may be added to bring performance back up to specification.

In accordance with this invention, a method is provided for converting heat from a heat source to mechanical energy. This method comprises heating a working fluid using heat supplied from the heat source; and expanding the heated working fluid to lower the pressure of the working fluid and generate mechanical energy as the pressure of the working fluid is lowered. The method is characterized by using a working fluid comprising a HFO-1234ze(Z) composition disclosed herein. The method for converting heat from a heat source to mechanical energy is a power cycle and may be an organic Rankine cycle (ORC).

The method is provided for converting heat from a heat source to mechanical energy may be a may be a sub-critical power cycle in which the organic working fluid used in the cycle receives heat at a pressure lower than the critical pressure of the organic working fluid and the working fluid remains below its critical pressure throughout the entire cycle.

The method is provided for converting heat from a heat source to mechanical energy may be a trans-critical power cycle, in which the organic working fluid used in the cycle receives heat at a pressure higher than the critical pressure of the organic working fluid. In a trans-critical cycle, the working fluid is compressed to a pressure above its critical pressure prior to being heated, and then during expansion the working fluid pressure is reduced to below its critical pressure.

The method is provided for converting heat from a heat source to mechanical energy may be a super-critical power cycle. In a super critical cycle, the working fluid remains above its critical pressure for the complete cycle (e.g., compression, heating, expansion and cooling).

In another embodiment, the present invention relates to foam expansion agent compositions comprising HFO-1234ze(Z) for use in preparing foams. In other embodiments the invention provides foamable compositions, and preferably thermoset (like polyurethane, polyisocyanurate, or phenolic) foam compositions, and thermoplastic (like polystyrene, polyethylene, or polypropylene) foam compositions and method of preparing foams. In such foam embodiments, one or more of the HFO-1234ze(Z) compositions are included as a foam expansion agent in foamable compositions, which composition preferably includes one or more additional components capable of reacting and/or mixing and foaming under the proper conditions to form a foam or cellular structure.

In one embodiment, the present invention further relates to a method of forming a foam comprising: (a) adding to a foamable composition a HFO-1234ze(Z) composition of the present invention; and (b) processing the foamable composition under conditions effective to form a foam.

Another embodiment of the present invention relates to the use of the HFO-1234ze(Z) compositions of the present invention as propellants in sprayable compositions. Additionally, the present invention relates to a sprayable composition comprising HFO-1234ze(Z). The active ingredient to be sprayed together with inert ingredients, solvents and other materials may also be present in a sprayable composition. In one embodiment, a sprayable composition is an aerosol. The present compositions can be used to formulate a variety of industrial aerosols or other sprayable compositions such as contact cleaners, dusters, lubricant sprays, mold release sprays, insecticides, and the like, and consumer aerosols such as personal care products (such as, e.g., hair sprays, deodorants, and perfumes), household products (such as, e.g., waxes, polishes, pan sprays, room fresheners, and household insecticides), and automotive products (such as, e.g., cleaners and polishers), as well as medicinal materials such as anti-asthma and anti-halitosis medications. Examples of this includes metered dose inhalers (MDIs) for the treatment of asthma and other chronic obstructive pulmonary diseases and for delivery of medicaments to accessible mucous membranes or intra-nasally.

The present invention further relates to a process for producing aerosol products comprising the step of adding a composition of the present invention comprising HFO-1234ze(Z) to a formulation, including active, ingredients in an aerosol container, wherein said composition functions as a propellant. Additionally, the present invention further relates to a process for producing aerosol products comprising the step of adding a composition of the present invention comprising HFO-1234ze(Z) to a barrier type aerosol package (like a bag-in-a-can or piston can) wherein said composition is kept separated from other formulation ingredients in an aerosol container, and wherein said composition functions as a propellant. Additionally, the present invention further relates to a process for producing aerosol products comprising the step of adding only a composition of the present invention comprising HFO-1234ze(Z) to an aerosol package, wherein said composition functions as the active ingredient (e.g., a duster, or a cooling or freezing spray).

Also provided is a method for detecting a leak from a container comprising sampling the air in the vicinity of the container and detecting at least one fluorinated compound with means for detecting the leak, wherein the composition of the present invention comprising HFO-1234ze(Z) is contained inside the container.

A container may be any known container or system or apparatus that is filled with a HFO-1234ze(Z) composition of the present invention. A container may include but is not limited to a storage container, a transport container, an aerosol can, a fire extinguishing system, a chiller apparatus, a heat pump apparatus, heat transfer container, and a power cycle apparatus (e.g., an organic Rankine cycle system).

Means for detecting a leak may be any known sensor designed to detect leaks. In particular, means for detecting the leak includes, but is not limited to, electrochemical, corona discharge and mass spectroscopic leak detectors.

By “in the vicinity of” the container is meant within 12 inches of the outside surface of the container. Alternatively, in the vicinity may be within 6 inches, within 3 inches or within one inch of the outside surface of the container.

In some embodiments, the HFO-1234ze(Z) compositions of the present invention may be used in a refrigeration system. One embodiment of a refrigeration system includes an evaporator, a condenser, a compressor, an expansion device, and a heat transfer media. The heat transfer media includes the HFO-1234ze(Z) compositions of the present invention.

In another embodiment, the HFO-1234ze(Z) compositions of the present invention may be used in a process to transfer heat. The process may include providing an article and contacting the article with a heat transfer media including the HFO-1234ze(Z) compositions of the present invention. In some embodiments, the article may include electrical equipment (e.g., circuit board, computer, display, semiconductor chip, or transformer), a heat transfer surface (e.g., heat sink), or article of clothing (e.g., a body suit).

In another embodiment, the present invention relates to blowing agent compositions comprising the HFO-1234ze(Z) compositions of the present invention.

In another embodiment, provided herein is a storage container for refrigerant containing the HFO-1234ze(Z) compositions of the present invention, wherein the refrigerant comprises gaseous and liquid phases.

Another embodiment of the invention relates to storing the foregoing HFO-1234ze(Z) compositions in gaseous and/or liquid phases within a sealed container. The container will be properly prepared for loading with the present compositions by evacuation and heating such that there are limits on the amount of water and/or oxygen to prevent reaction or degradation of the refrigerant portion of the compositions within the container. In one embodiment, the water is limited to 0.1 to 200 ppm by weight, or 0.1 to 100 ppm by weight, or 0.1 to 50 ppm by weight or 0.1 to 10 ppm by weight. In another embodiment, the oxygen is limited to 0.6 volume percent or less of the vapor phase. In another embodiment, the oxygen is present from about 0.01 to 0.35 volume percent. In yet another embodiment, the oxygen is limited to 0.01 to 0.25 volume percent. And in yet another embodiment, the oxygen is limited to 0.01 to 0.15 volume percent.

The container for storing the HFO-1234ze(Z) compositions of the present invention can be constructed of any suitable material and design that is capable of sealing the compositions therein while maintaining gaseous and liquids phases. Examples of suitable containers comprise pressure resistant containers such as a tank, a filling cylinder, and a secondary filling cylinder. The container can be constructed from any suitable material such as carbon steel, manganese steel, chromium-molybdenum steel, among other low-alloy steels, stainless steel and in some cases an aluminum alloy. The container can include a pierce top or valves suitable for dispensing flammable substances.

While any suitable method can be employed for stabilizing the HFO-1234ze(Z) compositions of the present invention, examples of such methods including blending the foregoing inhibitors with the HFO-1234ze(Z) compositions of the present invention, purging lines and containers with a material comprising the inhibitor (e.g., an inhibitor with a nitrogen carrier, or the inventive stabilized composition); among other suitable methods.

Another embodiment of the invention includes a refrigerant charging kit comprising the HFO-1234ze(Z) compositions of the present invention (which may be in the stabilized form) in a sealed canister, comprising HFO-1234ze(Z), optionally at least one compound selected from HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224 yd(E), CFO-1112(E), CFO-1112 (Z), HFC-245fa, HFC-236fa, HFC-227ea, trans-1,2-dichloroethylene, HFO-1132(E), HFO-1132 (Z), HFC-152a, HFC-134a, HFC-134, HFC-32, HFC-125, isobutene, propane, butane, isobutane, pentane and isopentane, and optionally carbon dioxide, and a tube for connecting a discharge end of the refrigerant canister to a valve of a refrigerant circuit. In certain embodiments, any of the above-mentioned additives can be included in the stabilized refrigerant blend. Thus, the refrigerant charging kit can include any of the disclosed refrigerant blends which have been stabilized, but without a lubricant.

In another embodiment, the present invention relates to processes for the reclamation of any of the foregoing compositions, such integrated processes being described in U.S. Provisional Application No. 63/402,727, titled “Liquid Reclamation and Solid Foam Recycling/Reclamation: Compositions and Methods”, filed on Aug. 31, 2022, and U.S. Provisional Application No. 63/422,656, titled “Integrated System and Process for Producing Reclaimed, Stabilized and Traceable Refrigerant Compositions”, filed on Nov. 4, 2022, the entire contents of both of which are incorporated herein in their entireties.

For example, in one embodiment, provided herein is a process comprising the following steps: a) providing an unreclaimed refrigerant comprising at least HFO-Z-1234ze; testing the unreclaimed refrigerant composition comprising at least HFO-Z-1234ze and which may further comprise contaminants, non-condensable gases (NCG), and physical properties; checking and comparing the purity of the unreclaimed refrigerant composition relative to AHRI 700 standards; and if the unreclaimed refrigerant composition of does not meet AHRI 700 standards, treating and purifying the unreclaimed refrigerant composition and providing at least one first treated product; and optionally repeating the procedure on the first treated product if needed to meet AHRI 700 standards; and optionally adding additional refrigerant components to the first treated product to form a first target refrigerant or refrigerant blend if the first treated product meets or exceeds AHRI 700 standards, or (2) further purifying the first treated product does not meet AHRI 700 standards to produce a second treated product and repeating the procedure as needed to obtain a second treated product which meets or exceeds AHRI 700 standards.

In another embodiment, a system for heating and/or cooling is provided. The system comprises an evaporator, compressor, condenser, and expansion device. The system contains any of the compositions disclosed herein. In one embodiment, the system for heating and/or cooling may be a heat pump or a chiller.

### High Temperature Heat Pump Apparatus and Methods

In one preferred embodiment of the present invention is provided a heat pump apparatus containing a working fluid comprising any of the compositions disclosed herein comprising HFO-1234ze(Z), referred to herein as “HFO-1234ze(Z) composition” or “HFO-1234ze(Z) compositions”, either as a single component working fluid or as a working fluid blend. In one embodiment, the present invention relates to a method for producing heating and/or cooling in a heat pump utilizing any of the HFO-1234ze(Z) compositions of the present invention as the working fluid.

A heat pump is a type of apparatus for producing heating and/or cooling. A heat pump includes an evaporator, a compressor, a condenser or supercritical working fluid cooler, and an expansion device. A working fluid circulates through these components in a repeating cycle. Heating is produced at the condenser where energy (in the form of heat) is extracted from the vapor working fluid as it is condensed to form liquid working fluid. Cooling is produced at the evaporator where energy is absorbed to evaporate the working fluid to form vapor working fluid.

In one embodiment, the high temperature heat pump apparatus of the present invention comprises (a) an evaporator through which a working fluid flows and is evaporated; (b) a compressor in fluid communication with the evaporator that compresses the evaporated working fluid to a higher pressure; (c) a condenser in fluid communication with the compressor through which the high pressure working fluid vapor flows and is condensed; and (d) a pressure reduction device in fluid communication with the condenser wherein the pressure of the condensed working fluid is reduced and said pressure reduction device further being in fluid communication with the evaporator such that the working fluid then repeats flow through components (a), (b), (c) and (d) in a repeating cycle.

A heat pump may be a residential heat pump for heating air. Residential heat pumps are used to produce heated air to warm a residence or home (including single family or multi-unit attached homes) and produce maximum condenser operating temperatures from about 30° C. to about 50° C. In another embodiment, a heat pump may be a high temperature heat pump, by which is meant a heat pump with condenser temperatures above 55° C., or with condenser temperatures above 80° C., or even with condenser temperatures above 100° C.

In one embodiment, the present invention relates to a method for producing heating in a high temperature heat pump comprising condensing a vapor working fluid comprising the HFO-1234ze(Z) composition, in a condenser, thereby producing a liquid working fluid. The high temperature heat pump may operate at a condenser temperature of at least about 100° C. The high temperature heat pump may comprise a centrifugal compressor or positive displacement compressor.

In some embodiments, a method and system are provided for producing heating in a high temperature heat pump having a condenser wherein a vapor working fluid is condensed to heat a heat transfer medium and the heated heat transfer medium is transported out of the condenser to a body to be heated. The method comprises condensing a vapor working fluid in a condenser, thereby producing a liquid working fluid wherein said vapor and liquid working fluid comprises any of the present compositions comprising HFO-1234ze(Z).

In one embodiment is provided a method for producing heating in a high temperature heat pump comprising extracting heat from a working fluid, thereby producing a cooled working fluid wherein said working fluid comprises any of the present compositions comprising HFO-1234ze(Z).

Heat pumps may include flooded evaporators or direct expansion evaporators.

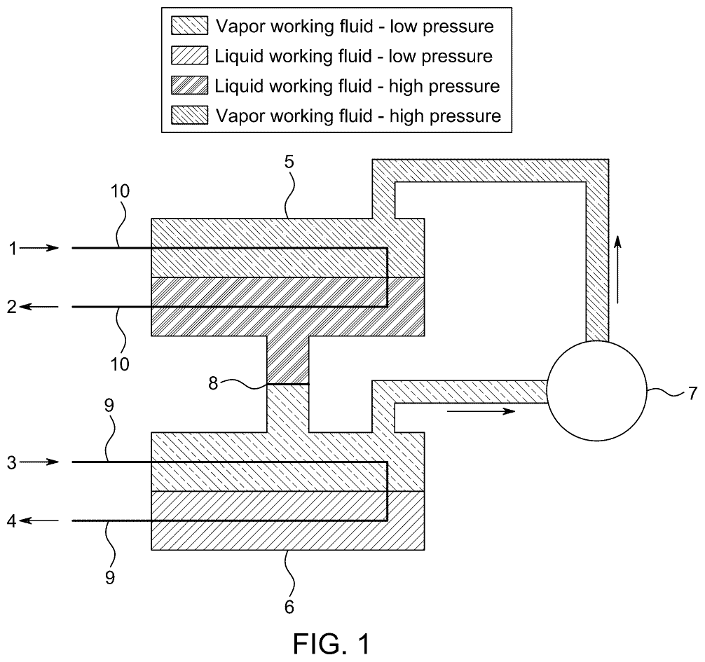

In one embodiment, the method for producing heating comprises extracting heat in a flooded evaporator high temperature heat pump, as shown in FIG. 1. In this method, the liquid working fluid is evaporated to form a working fluid vapor in the vicinity of a first heat transfer medium. The first heat transfer medium is a warm liquid, such as water, which is transported into the evaporator via a pipe from a low temperature heat source. The warm liquid is cooled and is returned to the low temperature heat source or is passed to a body to be cooled, such as a building. The working fluid vapor is then condensed in the vicinity of a second heat transfer medium, which is a chilled liquid which is brought in from the vicinity of a body to be heated (heat sink). The second heat transfer medium cools the working fluid such that it is condensed to form a liquid working fluid. In this method, a flooded evaporator heat pump may also be used to heat domestic or service water or a process stream.

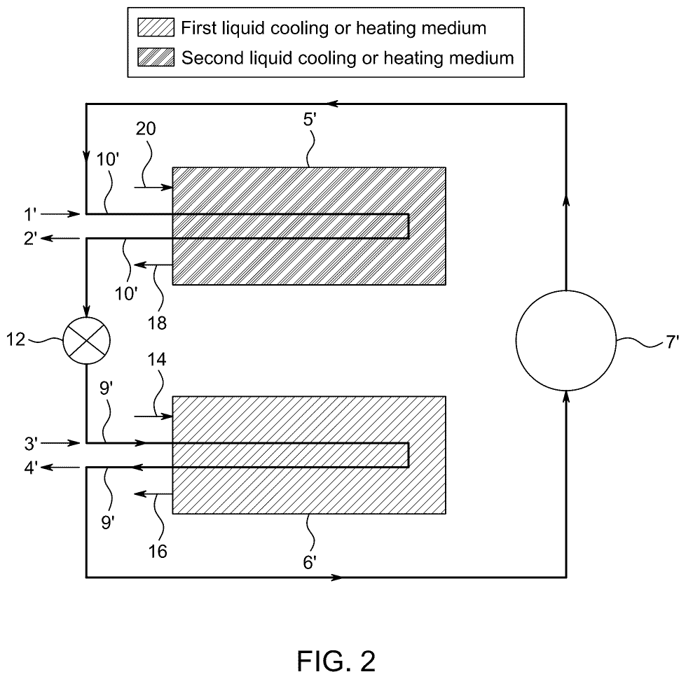

In another embodiment, the method for producing heating comprises producing heating in a direct expansion high temperature heat pump, as shown in FIG. 2. In this method, the liquid working fluid is passed through an evaporator and evaporates to produce a working fluid vapor. A first liquid heat transfer medium is cooled by the evaporating working fluid. The first liquid heat transfer medium is passed out of the evaporator to a low temperature heat source or a body to be cooled. The working fluid vapor is then condensed in the vicinity of a second heat transfer medium, which is a chilled liquid which is brought in from the vicinity of a body to be heated (heat sink). The second heat transfer medium cools the working fluid such that it is condensed to form a liquid working fluid. In this method, a direct expansion heat pump may also be used to heat domestic or service water or a process stream.

In one embodiment of the method for producing heating, the high temperature heat pump includes a compressor which is a centrifugal compressor.

In one embodiment of the method for producing heat, heat is exchanged between at least two heating stages, the method comprises absorbing heat in a working fluid in a heating stage operated at a selected condensing temperature and transferring this heat to the working fluid of another heating stage operated at a higher condensing temperature; wherein the working fluid of the heating stage operated at the higher condensing temperature comprises any of the compositions disclosed herein comprising HFO-1234ze(Z).

In one embodiment, a method for producing heating in a high temperature heat pump is provided, wherein heat is exchanged between at least two stages arranged in a cascade configuration, comprising absorbing heat at a selected lower temperature in a first working fluid in a first cascade stage and transferring this heat to a second working fluid of a second cascade stage that supplies heat at a higher temperature; wherein the second working fluid comprises any of the compositions disclosed herein comprising HFO-1234ze(Z). In another embodiment, the heat supplied in the second cascade stage is at a temperature of at least 150° C.

In another embodiment of the invention is disclosed a method of raising the condenser operating temperature in a high temperature heat pump apparatus comprising charging the high temperature heat pump with a working fluid comprising any of the compositions disclosed herein comprising HFO-1234ze(Z). In one embodiment of the method to raise the condenser operating temperature, when any of compositions disclosed herein comprising HFO-1234ze(Z) is used as the heat pump working fluid, the condenser operating temperature is raised to a temperature greater than about 150° C., greater than about 160° C., greater than about 170° C., or even greater than about 180° C.

It may be feasible that temperatures as high as, for example, 175° C. are achievable with a high temperature heat pump utilizing any of compositions disclosed herein comprising HFO-1234ze(Z) as working fluid. However, at temperatures above 175° C., some modification of the high-pressure heat exchanger or its materials, may be necessary.

In some embodiments, the present compositions comprising R-1234zeZ are useful as the working fluid for large lift high temperature heat pump systems. In one embodiment, compared to an incumbent fluid, such as R-1233zdE and many other pure components with a large enough critical temperature to deliver greater than 120° C. heat at a 130° C. condensing temperature, R-1234zeZ allows for a large 80° C. temperature lift in flooded high temperature heat pump systems while also avoiding wet compression without suction gas superheat. R-1233zdE, on the other hand, is not capable of avoiding wet compression of the compressor discharge gas.

In one embodiment, the high temperature heat pump apparatus uses a working fluid comprising the HFO-1234ze(Z) composition. Heat pumps may include flooded evaporators one embodiment of which is shown in FIG. 1, or direct expansion evaporators one embodiment of which is shown in FIG. 2.

Of note are high temperature heat pumps that may be used to heat air, water, another heat transfer medium or some portion of an industrial process, such as a piece of equipment, storage area or process stream. In one embodiment, these high temperature heat pumps use condenser operating temperatures greater than about 55° C. In one embodiment, the condenser operating temperature for a high temperature heat pump is from about 55° C. to about 150° C. In one embodiment, the condenser operating temperature for a high temperature heat pump is from about 55° C. to about 130° C. In another embodiment, the condenser operating temperature for a high temperature heat pump, particularly for heating water, is from about 130° C. to about 150° C. In one embodiment, the system for heating may be a water heating heat pump. The maximum condenser operating temperature that can be achieved in a high temperature heat pump will depend upon the working fluid used. This maximum condenser operating temperature is limited by the normal boiling characteristics of the working fluid and also by the pressure to which the heat pump's compressor can raise the vapor working fluid pressure. This maximum pressure is also related to the working fluid used in the heat pump.

In some embodiments, the HFO-1234ze(Z) composition used in the high temperature heat pump system has a critical temperature which is at least 5° C. greater than an operating temperature of the condenser. In one embodiment, the HFO-1234ze(Z) composition used in the high temperature heat pump system has a critical temperature of at least about 60° C., preferably at least about 135° C., and most preferably at least about 155° C.

Also of note are heat pumps that are used to produce heating and cooling simultaneously. For instance, a single heat pump unit may produce heating to be used to generate high temperature steam for industrial use and may also produce cooling to be used to cool an industrial process stream.

Heat pumps, including both flooded evaporator and direct expansion, may be coupled with an air handling and distribution system to provide drying and dehumidification. In another embodiment, heat pumps may be used to heat water or generate steam.

Referring to FIG. 1, there is shown a flooded evaporator high temperature heat pump using a working fluid comprising any of the HFO-1234ze(Z) compositions of the present invention. In this heat pump, a second heat transfer medium, which in some embodiments is a warm liquid, which may comprise water, and, in some embodiments, additives, or other heat transfer medium such as a glycol (e.g., ethylene glycol or propylene glycol), enters the heat pump carrying heat from a low temperature source (not shown), such as for instance, an industrial vessel or process stream, shown entering the heat pump at arrow 3, through a tube bundle or coil 9, in an evaporator 6, which has an inlet and an outlet. The warm second heat transfer medium is delivered to evaporator 6, where it is cooled by liquid working fluid, which is shown in the lower portion of evaporator 6. The liquid working fluid evaporates at a lower temperature than the warm first heat transfer medium which flows through tube bundle or coil 9. The cooled second heat transfer medium re-circulates back to the low temperature heat source as shown by arrow 4, via a return portion of tube bundle or coil 9. The liquid working fluid, shown in the lower portion of evaporator 6 in FIG. 1, vaporizes and is drawn into compressor 7, which increases the pressure and temperature of the working fluid vapor. Compressor 7 compresses this vapor so that it may be condensed in condenser 5 at a higher pressure and temperature than the pressure and temperature of the working fluid vapor when it exits evaporator 6. A first heat transfer medium enters the condenser via a tube bundle or coil 10 in condenser 5 from a location where high temperature heat is provided (“heat sink”) such as a service water heater or a steam generation system at arrow 1 in FIG. 1. The first heat transfer medium is warmed in the process and returned via a return loop of tube bundle or coil 10 and arrow 2 to the heat sink. This first heat transfer medium cools the working fluid vapor in condenser 5 and causes the vapor to condense to liquid working fluid, so that there is liquid working fluid in the lower portion of condenser 5 as shown in FIG. 1. Condensed liquid working fluid in condenser 5 flows back to evaporator 6 through expansion device 8, which may be an orifice, capillary tube, or expansion valve. Expansion device 8 reduces the pressure of the liquid working fluid and converts the liquid working fluid at least partially to vapor, that is to say that the liquid working fluid flashes as pressure drops between condenser 5 and evaporator 6. Flashing cools the working fluid, i.e., both the liquid working fluid and the working fluid vapor to the saturated temperature at evaporator pressure, so that both liquid working fluid and working fluid vapor are present in evaporator 6.

In some embodiments the working fluid vapor is compressed to a supercritical state and condenser 5 is replaced by a gas cooler where the working fluid vapor is cooled to a liquid state without condensation.

In some embodiments, the second heat transfer medium used in the apparatus depicted in FIG. 1 is a medium returning from a location where cooling is provided to a stream or a body to be cooled. Heat is extracted from the returning second heat transfer medium at the evaporator 6 and the cooled second heat transfer medium is supplied back to the location or body to be cooled. In this embodiment the apparatus depicted in FIG. 1 functions to simultaneously cool the second heat transfer medium that provides cooling to a body to be cooled (e.g., a process stream) and heat the first heat transfer medium that provides heating to a body to be heated (e.g., service water or steam or a process stream).

It is understood that the apparatus depicted in FIG. 1 can extract heat at the evaporator 6 from a wide variety of heat sources including solar, geothermal and waste heat and supply heat from the condenser 5 to a wide range of heat sinks.

It should be noted that for a single component working fluid composition, the composition of the vapor working fluid in the evaporator and condenser is the same as the composition of the liquid working fluid in the evaporator and condenser. In this case, evaporation will occur at a constant temperature. However, if a working fluid blend (or mixture) is used, the liquid working fluid and the working fluid vapor in the evaporator (or in the condenser) may have different compositions. This may lead to inefficient systems and difficulties in servicing the equipment. An azeotrope or azeotrope-like composition will function essentially as a single component working fluid in a heat pump, such that the liquid composition and the vapor composition are essentially the same reducing any inefficiencies that might arise from the use of a non-azeotropic or non-azeotrope-like composition. The above discussion notwithstanding, in some embodiments, zeotropic working fluids may be advantageous in creating condenser and/or evaporator temperature glides that largely match the temperature variations in the heat sink and/or heat source, respectively, so as to increase the effectiveness of heat exchange between the working fluid and the sink and/or source.

One embodiment of a direct expansion heat pump using a working fluid comprising any of the HFO-1234ze(Z) compositions of the present invention is illustrated in FIG. 2. In the heat pump as illustrated in FIG. 2, liquid second heat transfer medium, which in some embodiments is a warm liquid, such as warm water, enters evaporator 6′ at inlet 14. Mostly liquid working fluid (with a small amount of working fluid vapor) enters coil 9′ in the evaporator at arrow 3′ and evaporates. As a result, second liquid heat transfer medium is cooled in evaporator 6′, and a cooled second liquid heat transfer medium exits evaporator 6′ at outlet 16 and is sent to low temperature heat source (e.g., warm water flowing to a cooling tower). The working fluid vapor exits evaporator 6′ at arrow 4′ and is sent to compressor 7′, where it is compressed and exits as high temperature, high pressure working fluid vapor. This working fluid vapor enters condenser 5′ through condenser coil 10′ at 1′. The working fluid vapor is cooled by a liquid first heat transfer medium, such as water, in condenser 5′ and becomes a liquid. The liquid first heat transfer medium enters condenser 5′ through condenser heat transfer medium inlet 20. The liquid first heat transfer medium extracts heat from the condensing working fluid vapor, which becomes liquid working fluid, and this warms the liquid first heat transfer medium in condenser 5′. The liquid first heat transfer medium exits from condenser 5′ through condenser heat transfer medium outlet 18. The condensed working fluid exits condenser 5′ through lower coil or tube bundle 10′ as shown in FIG. 2 and flows through expansion device 12, which may be an orifice, capillary tube or expansion valve. Expansion device 12 reduces the pressure of the liquid working fluid. A small amount of vapor, produced as a result of the expansion, enters evaporator 6′ with liquid working fluid through coil 9′ and the cycle repeats.

In some embodiments the working fluid vapor is compressed to a supercritical state and vessel 5′ in FIG. 2 represents a gas cooler where the working fluid vapor is cooled to a liquid state without condensation.

In some embodiments the first liquid heating medium used in the apparatus depicted in FIG. 2 is a medium returning from a location where cooling is provided to a stream or a body to be cooled. Heat is extracted from the returning second heat transfer medium at the evaporator 6′ and the cooled second heat transfer medium is supplied back to the location or body to be cooled. In this embodiment the apparatus depicted in FIG. 2 functions to simultaneously cool the second heat transfer medium (may be referred to as a liquid heating medium since it provides heating to the working fluid) that provides cooling to a body to be cooled (e.g., a process stream) and heat the first heat transfer medium (or liquid heating medium) that provides heating to a body to be heated (e.g., service water or process stream).

It is understood that the apparatus depicted in FIG. 2 can extract heat at the evaporator 6′ from a wide variety of heat sources including solar, geothermal and waste heat and supply heat from the condenser 5′ to a wide range of heat sinks.

Compressors useful in the present invention include dynamic compressors. Of note as examples of dynamic compressors are centrifugal compressors. A centrifugal compressor uses rotating elements to accelerate the working fluid radially, and typically includes an impeller and diffuser housed in a casing. Centrifugal compressors usually take working fluid in at an impeller eye, or central inlet of a circulating impeller, and accelerate it radially outward. Some static pressure rise occurs in the impeller, but most of the pressure rise occurs in the diffuser section of the casing, where velocity is converted to static pressure. Each impeller-diffuser set is a stage of the compressor. Centrifugal compressors are built with from 1 to 12 or more stages, depending on the final pressure desired and the volume of refrigerant to be handled.

The pressure ratio, or compression ratio, of a compressor is the ratio of absolute discharge pressure to the absolute inlet pressure. Pressure delivered by a centrifugal compressor is practically constant over a relatively wide range of capacities. The pressure a centrifugal compressor can develop depends on the tip speed of the impeller. Tip speed is the speed of the impeller measured at its tip and is related to the diameter of the impeller and its revolutions per minute The tip speed required in a specific application depends on the compressor work that is required to elevate the thermodynamic state of the working fluid from evaporator to condenser conditions. The volumetric flow capacity of the centrifugal compressor is determined by the size of the passages through the impeller. This makes the size of the compressor more dependent on the pressure required than the volumetric flow capacity required.

Also of note as examples of dynamic compressors are axial compressors. A compressor in which the fluid enters and leaves in the axial direction is called an axial flow compressor. Axial compressors are rotating, airfoil- or blade-based compressors in which the working fluid principally flows parallel to the axis of rotation. This is in contrast with other rotating compressors such as centrifugal or mixed-flow compressors where the working fluid may enter axially but will have a significant radial component on exit. Axial flow compressors produce a continuous flow of compressed gas and have the benefits of high efficiencies and large mass flow capacity, particularly in relation to their cross-section. They do, however, require several rows of airfoils to achieve large pressure rises making them complex and expensive relative to other designs.

Compressors useful in the present invention also include positive displacement compressors. Positive displacement compressors draw vapor into a chamber, and the chamber decreases in volume to compress the vapor. After being compressed, the vapor is forced from the chamber by further decreasing the volume of the chamber to zero or nearly zero.

Of note as examples of positive displacement compressors are reciprocating compressors. Reciprocating compressors use pistons driven by a crankshaft. They can be either stationary or portable, can be single or multi-staged, and can be driven by electric motors or internal combustion engines. Small reciprocating compressors from 5 to 30 hp are seen in automotive applications and are typically for intermittent duty. Larger reciprocating compressors up to 100 hp are found in large industrial applications. Discharge pressures can range from low pressure to very high pressure (above 5000 psi or 35 MPa).

Also of note as examples of positive displacement compressors are screw compressors. Screw compressors use two meshed rotating positive-displacement helical screws to force the gas into a smaller space. Screw compressors are usually for continuous operation in commercial and industrial application and may be either stationary or portable. Their application can be from 5 hp (3.7 kW) to over 500 hp (375 KW) and from low pressure to very high pressure (above 1200 psi or 8.3 MPa).

Also of note as examples of positive displacement compressors are scroll compressors. Scroll compressors are similar to screw compressors and include two interleaved spiral-shaped scrolls to compress the gas. The output is more pulsed than that of a rotary screw compressor.

In one embodiment, the high temperature heat pump apparatus may comprise more than one heating circuit (or loop or stage) in a cascade arrangement. The performance (coefficient of performance for heating and volumetric heating capacity) of high temperature heat pumps operated with the HFO-1234ze(Z) composition as the working fluid is drastically improved when the evaporator is operated at temperatures approaching the condenser temperature required by the application. When the heat supplied to the evaporator is only available at low temperatures, thus requiring high temperature lifts leading to poor performance, a cascade cycle configuration with multiple circuits (or loops or stages) will be advantageous. The working fluid used in each cascade circuit (or loop or stage) is selected to have optimum thermodynamic and chemical stability properties for the temperature range encountered in the cascade circuit or stage in which the fluid is used.

In one embodiment of a cascade heat pump, the heat pump has two circuits or stages. In one embodiment, the low stage or low temperature circuit of the cascade cycle with two circuits or stages may be operated with a working fluid of lower boiling point than the boiling point of the working fluid used in the upper or high stage. In one embodiment, the high stage or high temperature circuit of the cascade cycle may be operated with a working fluid comprising the HFO-1234ze(Z) composition. In another embodiment, the low stage or low temperature circuit of the cascade cycle may be operated with a working fluid comprising the HFO-1234ze(Z) composition.

In another embodiment of a cascade heat pump, the heat pump has three circuits or stages. When the heat supplied to the evaporator is only available at even lower temperatures than in the previous example, thus requiring high temperature lifts leading to poor performance, a cascade cycle configuration with three stages or three circuits will be advantageous. In one embodiment, the lowest stage or lowest temperature circuit of the cascade cycle may be operated with a working fluid of lower boiling point than the boiling point of the working fluid used in the second or intermediate stage. In one embodiment, the high stage or high temperature circuit of the cascade cycle may be operated with a working fluid comprising the HFO-1234ze(Z) composition. In one embodiment, the intermediate stage or intermediate temperature circuit of the cascade cycle may be operated with a working fluid comprising the HFO-1234ze(Z) composition. In one embodiment, the low stage or low temperature circuit of the cascade cycle would be operated with a working fluid comprising the HFO-1234ze(Z) composition.

The evaporator of the low temperature circuit (or low temperature loop) of the two-stage cascade cycle receives the available low temperature heat, lifts the heat to a temperature intermediate between the temperature of the available low temperature heat and the temperature of the required heating duty and transfers the heat to the high stage or high temperature circuit (or high temperature loop) of the cascade system at a cascade heat exchanger. Then the high temperature circuit, operated with a working fluid comprising the HFO-1234ze(Z) composition, further lifts the heat received at the cascade heat exchanger to the required condenser temperature to meet the intended heating duty. The cascade concept can be extended to configurations with three or more circuits lifting heat over wider temperature ranges and using different fluids over different temperature sub-ranges to optimize performance.

In accordance with the present invention, there is provided a cascade heat pump system having at least two heating loops for circulating a working fluid through each loop. In one embodiment, the high temperature heat pump apparatus has at least two heating stages arranged as a cascade heating system, wherein each stage is in thermal communication with the next stage and wherein each stage circulates a working fluid therethrough, wherein heat is transferred to the final or upper or highest-temperature stage from the immediately preceding stage and wherein the heating fluid of the final stage comprises the HFO-1234ze(Z) composition.

In another embodiment the high temperature heat pump apparatus has at least two heating stages arranged as a cascade heating system, each stage circulating a working fluid therethrough comprising (a) a first expansion device for reducing the pressure and temperature of a first working fluid liquid; (b) an evaporator in fluid communication with the first expansion device having an inlet and an outlet; (c) a first compressor in fluid communication with the evaporator and having an inlet and an outlet; (d) a cascade heat exchanger system in fluid communication with the first compressor and having: (i) a first inlet and a first outlet, and (ii) a second inlet and a second outlet in thermal communication with the first inlet and outlet; (e) a second compressor in fluid communication with the second outlet of the cascade heat exchanger and having an inlet and an outlet; (f) a condenser in fluid communication with the second compressor and having an inlet and an outlet; and (g) a second expansion device in fluid communication with the condenser; wherein the second working fluids comprises the HFO-1234ze(Z) composition.

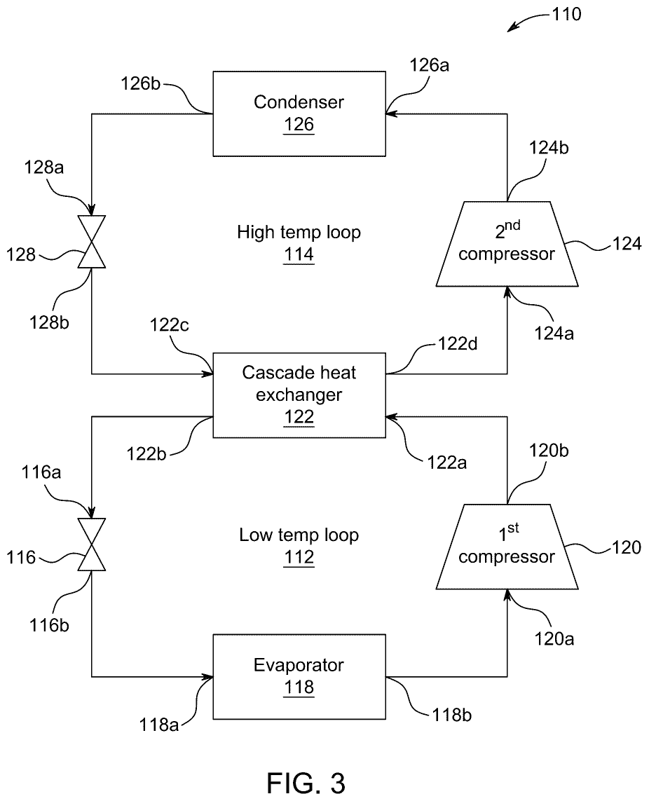

In accordance with the present invention, there is provided a cascade heat pump system having at least two heating loops for circulating a working fluid through each loop. One embodiment of such a cascade system is shown generally at 110 in FIG. 3. Cascade heat pump system 110 of the present invention has at least two heating loops, including a first, or lower loop 112, which is a low temperature loop, and a second, or upper loop 114, which is a high temperature loop 114 as shown in FIG. 3. Each circulates a working fluid therethrough.

Cascade heat pump system 110 includes first expansion device 116. First expansion device 116 has an inlet 116a and an outlet 116b. First expansion device 116 reduces the pressure and temperature of a first working fluid liquid which circulates through the first or low temperature loop 112.

Cascade heat pump system 110 also includes evaporator 118. Evaporator 118 has an inlet 118a and an outlet 118b. The first working fluid liquid from first expansion device 116 enters evaporator 118 through evaporator inlet 118a and is evaporated in evaporator 118 to form a first working fluid vapor. The first working fluid vapor then circulates to evaporator outlet 118b.

Cascade heat pump system 110 also includes first compressor 120. First compressor 120 has an inlet 120a and an outlet 120b. The first working fluid vapor from evaporator 118 circulates to inlet 120 a of first compressor 120 and is compressed, thereby increasing the pressure and the temperature of the first working fluid vapor. The compressed first working fluid vapor then circulates to the outlet 120b of the first compressor 120.

Cascade heat pump system 110 also includes cascade heat exchanger system 122. Cascade heat exchanger 122 has a first inlet 122a and a first outlet 122b. The first working fluid vapor from first compressor 120 enters first inlet 122a of heat exchanger 122 and is condensed in heat exchanger 122 to form a first working fluid liquid, thereby rejecting heat. The first working fluid liquid then circulates to first outlet 122b of heat exchanger 122. Heat exchanger 122 also includes a second inlet 122c and a second outlet 122d. A second working fluid liquid circulates from second inlet 122c to second outlet 122d of heat exchanger 122 and is evaporated to form a second working fluid vapor, thereby absorbing the heat rejected by the first working fluid (as it is condensed). The second working fluid vapor then circulates to second outlet 122d of heat exchanger 122. Thus, in the embodiment of FIG. 3, the heat rejected by the first working fluid is directly absorbed by the second working fluid.

Cascade heat pump system 110 also includes second compressor 124. Second compressor 124 has an inlet 124a and an outlet 124b. The second working fluid vapor from cascade heat exchanger 122 is drawn into compressor 124 through inlet 124a and is compressed, thereby increasing the pressure and temperature of the second working fluid vapor. The second working fluid vapor then circulates to outlet 124b of second compressor 124.

Cascade heat pump system 110 also includes condenser 126 having an inlet 126a and an outlet 126b. The second working fluid from second compressor 124 circulates from inlet 126a and is condensed in condenser 126 to form a second working fluid liquid, thus producing heat. The second working fluid liquid exits condenser 126 through outlet 126b.

Cascade heat pump system 110 also includes second expansion device 128 having an inlet 128a and an outlet 128b. The second working fluid liquid passes through second expansion device 128, which reduces the pressure and temperature of the second working fluid liquid exiting condenser 126. This liquid may be partially vaporized during this expansion. The reduced pressure and temperature second working fluid liquid circulates to second inlet 122c of cascade heat exchanger system 122 from expansion device 128.

Moreover, the stability of the HFO-1234ze(Z) at temperatures higher than its critical temperatures enables the design of heat pumps operated according to a supercritical or transcritical cycle in which heat is rejected by the working fluid in a supercritical state and made available for use over a range of temperatures. The supercritical fluid is cooled to a liquid state without passing through an isothermal condensation transition.

For high temperature condenser operation (associated with high temperature lifts and high compressor discharge temperatures) formulations of working fluid and lubricants with high thermal stability (possibly in combination with oil cooling or other mitigation approaches such as fluid injection during the compression stage) will be advantageous.

For high temperature condenser operation (associated with high temperature lifts and high compressor discharge temperatures) the use of magnetic centrifugal compressors (e.g., Danfoss-Turbocor type) that do not require the use of lubricants will be advantageous.

For high temperature condenser operation (associated with high temperature lifts and high compressor discharge temperatures) the use of compressor materials (e.g., shaft seals, etc.) with high thermal stability may also be required.

The HFO-1234ze(Z) composition may be used in a high temperature heat pump apparatus in combination with molecular sieves to aid in removal of moisture. Desiccants may be composed of activated alumina, silica gel, or zeolite-based molecular sieves. In some embodiments, the molecular sieves are most useful with a pore size of approximately 3 Angstroms to 6 Angstroms. Representative molecular sieves include MOLSIV XH-7, XH-6, XH-9 and XH-11 (UOP LLC, Des Plaines, Ill.).

### Chillers—Apparatuses and Methods

In one embodiment of the present invention is provided a chiller apparatus containing any of the HFO-1234ze(Z) compositions disclosed herein, either as a single component working fluid or as a working fluid blend. In one embodiment, the present invention relates to a method for producing heating and/or cooling in a chiller utilizing any of the HFO-1234ze(Z) compositions of the present invention as the working fluid.

A chiller is a type of air conditioning/refrigeration apparatus. The present disclosure is directed to a vapor compression chiller. Such vapor compression chillers may be either flooded evaporator chillers, one embodiment of which is shown in FIG. 1, or direct expansion chillers, one embodiment of which is shown in FIG. 2. Both a flooded evaporator chiller and a direct expansion chiller may be air-cooled or water-cooled. In the embodiment where chillers are water cooled, such chillers are generally associated with cooling towers for heat rejection from the system. In the embodiment where chillers are air-cooled, the chillers are equipped with refrigerant-to-air finned-tube condenser coils and fans to reject heat from the system. Air-cooled chiller systems are generally less costly than equivalent-capacity water-cooled chiller systems including cooling tower and water pump. However, water-cooled systems can be more efficient under many operating conditions due to lower condensing temperatures.

Chillers, including both flooded evaporator and direct expansion chillers, may be coupled with an air handling and distribution system to provide comfort air conditioning (cooling and dehumidifying the air) to large commercial buildings, including hotels, office buildings, hospitals, universities and the like. In another embodiment, chillers, most likely air-cooled direct expansion chillers, have found additional utility in naval submarines and surface vessels.

To illustrate how chillers operate, reference is made to FIGS. 1-2. A water-cooled, flooded evaporator chiller is shown illustrated in FIG. 1. In this chiller, a first cooling medium, which is a warm liquid, which comprises water, and, in some embodiments, additives, such as a glycol (e.g., ethylene glycol or propylene glycol), enters the chiller from a cooling system, such as a building cooling system, shown entering at arrow 3, through a coil 9, in an evaporator 6, which has an inlet and an outlet. The warm first cooling medium is delivered to the evaporator, where it is cooled by liquid refrigerant, which is shown in the lower portion of the evaporator. The liquid refrigerant evaporates at a lower temperature than the warm first cooling medium which flows through coil 9. The cooled first cooling medium re-circulates back to the building cooling system, as shown by arrow 4, via a return portion of coil 9. The liquid refrigerant, shown in the lower portion of evaporator 6 in FIG. 1, vaporizes and is drawn into a compressor 7, which increases the pressure and temperature of the refrigerant vapor. The compressor compresses this vapor so that it may be condensed in a condenser 5 at a higher pressure and temperature than the pressure and temperature of the refrigerant vapor when it comes out of the evaporator. A second cooling medium, which is a liquid in the case of a water-cooled chiller, enters the condenser via a coil 10 in condenser 5 from a cooling tower at arrow 1 in FIG. 1. The second cooling medium is warmed in the process and returned via a return loop of coil 10 and arrow 2 to a cooling tower or to the environment. This second cooling medium cools the vapor in the condenser and causes the vapor to condense to liquid refrigerant, so that there is liquid refrigerant in the lower portion of the condenser as shown in FIG. 1. The condensed liquid refrigerant in the condenser flows back to the evaporator through an expansion device 8, which may be an orifice, capillary tube or expansion valve. Expansion device 8 reduces the pressure of the liquid refrigerant, and converts the liquid refrigerant partially to vapor, that is to say that the liquid refrigerant flashes as pressure drops between the condenser and the evaporator. Flashing cools the refrigerant, i.e., both the liquid refrigerant and the refrigerant vapor to the saturated temperature at evaporator pressure, so that both liquid refrigerant and refrigerant vapor are present in the evaporator.

Chillers with cooling capacities above 700 KW generally employ flooded evaporators, where the refrigerant in the evaporator and the condenser surrounds a coil or other conduit for the cooling medium (i.e., the refrigerant is on the shell side). Flooded evaporators require higher charges of refrigerant but permit closer approach temperatures and higher efficiencies. Chillers with capacities below 700 KW commonly employ evaporators with refrigerant flowing inside the tubes and cooling medium in the evaporator and the condenser surrounding the tubes, i.e., the cooling medium is on the shell side. Such chillers are called direct-expansion (DX) chillers. One embodiment of a water-cooled direct expansion chiller is illustrated in FIG. 2. In the chiller as illustrated in FIG. 2, first liquid cooling medium, which is a warm liquid, such as warm water, enters an evaporator 6′ at inlet 14. Mostly liquid refrigerant (with a small amount of refrigerant vapor) enters a coil 9′ in the evaporator at arrow 3′ and evaporates, turning to vapor. As a result, first liquid cooling medium is cooled in the evaporator, and a cooled first liquid cooling medium exits the evaporator at outlet 16, and is sent to a body to be cooled, such as a building. In this embodiment of FIG. 2, it is this cooled first liquid cooling medium that cools the building or other body to be cooled. The refrigerant vapor exits the evaporator at arrow 4′ and is sent to a compressor 7′, where it is compressed and exits as high temperature, high pressure refrigerant vapor. This refrigerant vapor enters a condenser 5′ through a condenser coil 10′ at 1′. The refrigerant vapor is cooled by a second liquid cooling medium, such as water, in the condenser and becomes a liquid. The second liquid cooling medium enters the condenser through a condenser cooling medium inlet 20. The second liquid cooling medium extracts heat from the condensing refrigerant vapor, which becomes liquid refrigerant, and this warms the second liquid cooling medium in the condenser. The second liquid cooling medium exits through the condenser through the condenser cooling medium outlet 18. The condensed refrigerant liquid exits the condenser through lower coil 10′ as shown in FIG. 2 and flows through an expansion device 12, which may be an orifice, capillary tube or expansion valve. Expansion device 12 reduces the pressure of the liquid refrigerant. A small amount of vapor, produced as a result of the expansion, enters the evaporator with liquid refrigerant through coil 9′ and the cycle repeats.

Vapor-compression chillers may be identified by the type of compressor they employ. In one embodiment, the HFO-1234ze(Z) compositions of the present invention are useful in a chiller which utilizes a centrifugal compressor, hereinafter referred to as a centrifugal chiller, as will be described below. Thus, in one embodiment is provided a centrifugal chiller containing the HFO-1234ze(Z) compositions of the present invention.

In one embodiment, the method for producing cooling comprises producing cooling in a flooded evaporator chiller as described above with respect to FIG. 1. In this method, a HFO-1234ze(Z) composition of the present invention is evaporated to form a refrigerant vapor in the vicinity of a first cooling medium. The cooling medium is a warm liquid, such as water, which is transported into the evaporator via a pipe from a cooling system. The warm liquid is cooled and is passed to a body to be cooled, such as a building. The refrigerant vapor is then condensed in the vicinity of a second cooling medium, which is a chilled liquid which is brought in from, for instance, a cooling tower. The second cooling medium cools the refrigerant vapor such that it is condensed to form a liquid refrigerant. In this method, a flooded evaporator chiller may also be used to cool hotels, office buildings, hospitals and universities.

In another embodiment, the method for producing cooling comprises producing cooling in a direct expansion chiller as described above with respect to FIG. 2. In this method, a HFO-1234ze(Z) compositions of the present invention is passed through an evaporator and evaporates to produce a refrigerant vapor. A first liquid cooling medium is cooled by the evaporating refrigerant. The first liquid cooling medium is passed out of the evaporator to a body to be cooled. In this method, the direct expansion chiller may also be used to cool hotels, office buildings, hospitals, universities, as well as naval submarines or naval surface vessels.

In either method for producing cooling in either a flooded evaporator chiller or in direct expansion chiller, the chiller includes a compressor which is a centrifugal compressor.

It should be noted that for the flooded evaporator applications disclosed herein, with respect particularly to heat pump systems and chillers, for a single component refrigerant composition, the composition of the vapor refrigerant in the evaporator is the same as the composition of the liquid refrigerant in the evaporator. In this case, evaporation will occur at a constant temperature. However, if a refrigerant blend (or mixture) is used, the liquid refrigerant and the refrigerant vapor in the evaporator (or in the condenser) may have different compositions. Thus, where the system utilizes one of the blends disclosed herein, in use, the vapor phase refrigerant will have a different composition than the liquid phase refrigerant. The compositions disclosed above relate to the overall nominal composition in the system, which is close to the liquid composition in the evaporator.

Without further elaboration, it is believed that one skilled in the art can, using the description herein, utilize the present invention to its fullest extent. The following specific embodiments are, therefore, to be construed as merely illustrative, and do not constrain the remainder of the disclosure in any way whatsoever.

The invention will be described in greater detail below by way of specific examples. The following examples are offered for illustrative purposes and are not intended to limit the invention in any manner. Those of skill in the art will readily recognize a variety of non-critical parameters which can be changed or modified to yield essentially the same results.

### EXAMPLES

### Materials

The materials used to prepare the Examples are commercially available or may be prepared by known methods.

### Example 1: HFO-1234ze(Z) was Prepared as Follows

An Inconel® pipe (0.5-inch (1.27 cm) OD, 10-inch (25.4 cm) length, 0.35 in (0.89 cm wall thickness) was used as the reactor and was filled with 5 cc of fluorinated Cr₂O₃ catalyst (Louisville Cr). A flow of air (3.6 vol % O2) and CF₃CH₂CHF₂ (R-245fa) was fed at a rate 0.7 ml/hr over the catalyst bed at a temperature of 370° C. The contact time in the reactor was 47 seconds. The CF₃CH₂CHF₂ (R-245fa) was vaporized at a temperature of 80° C. Part of the reactor effluent was passed through a series of valves and analyzed by Agilent® 6890 GC/5975C MS and a Restek® PC2618 5% Krytox® CBK-D/60/80 6-meter×2 mm ID ⅛″ OD packed column purged with helium at 30 sccm. Samples were taken in hourly intervals. Compositions comprising HFO-1234ze(Z) were obtained, as shown in Table 4, with the amounts of components being expressed as mole percent.

TABLE 4
Example 1

| | Unknowns | 1141 | 143a | 152a | Trifluoropropyne |
|---|---|---|---|---|---|
| Inj | % | % | % | % | % |
| 1 | 1.12 | 0.03 | 0.52 | 0.00 | 0.51 |
| 2 | 0.16 | 0.04 | 0.37 | 0.00 | 0.57 |
| 3 | 0.15 | 0.04 | 0.35 | 0.00 | 0.58 |
| 4 | 0.15 | 0.04 | 0.33 | 0.00 | 0.58 |
| 5 | 0.14 | 0.04 | 0.31 | 0.00 | 0.58 |
| 6 | 0.13 | 0.03 | 0.28 | 0.00 | 0.59 |
| 7 | 0.11 | 0.03 | 0.27 | 0.00 | 0.60 |
| 8 | 0.12 | 0.03 | 0.28 | 0.00 | 0.59 |
| 9 | 0.10 | 0.03 | 0.25 | 0.00 | 0.61 |
| 10 | 0.05 | 0.03 | 0.24 | 0.00 | 0.59 |
| 11 | 0.05 | 0.03 | 0.25 | 0.00 | 0.60 |
| 12 | 0.12 | 0.04 | 0.30 | 0.00 | 0.61 |
| 13 | 0.20 | 0.06 | 0.35 | 0.00 | 0.63 |
| 14 | 0.20 | 0.06 | 0.35 | 0.00 | 0.63 |

| | 1234yf | E-1234ze | Z-1234ze | 245fa | E-1233zd | Z-1233zd |
|---|---|---|---|---|---|---|
| Inj | % | % | % | % | % | % |
| 1 | 0.93 | 70.84 | 18.47 | 6.78 | 0.66 | 0.13 |
| 2 | 0.68 | 71.12 | 19.22 | 7.05 | 0.66 | 0.14 |
| 3 | 0.64 | 71.12 | 19.25 | 7.06 | 0.66 | 0.14 |
| 4 | 0.56 | 71.29 | 19.19 | 7.06 | 0.65 | 0.15 |
| 5 | 0.53 | 71.32 | 19.23 | 7.04 | 0.66 | 0.15 |
| 6 | 0.46 | 71.36 | 19.28 | 7.07 | 0.65 | 0.14 |
| 7 | 0.39 | 71.41 | 19.39 | 7.00 | 0.66 | 0.14 |
| 8 | 0.46 | 71.38 | 19.31 | 7.03 | 0.65 | 0.14 |
| 9 | 0.32 | 71.60 | 19.34 | 6.94 | 0.67 | 0.14 |
| 10 | 0.33 | 71.66 | 19.28 | 7.09 | 0.60 | 0.13 |
| 11 | 0.36 | 71.61 | 19.41 | 7.07 | 0.51 | 0.11 |
| 12 | 0.45 | 71.66 | 19.26 | 6.91 | 0.54 | 0.12 |
| 13 | 0.56 | 71.52 | 19.30 | 6.68 | 0.57 | 0.13 |
| 14 | 0.55 | 71.60 | 19.25 | 6.67 | 0.56 | 0.13 |

### Example 2: HFO-1234ze(Z) was Prepared as Follows

An Inconel® pipe (0.5-inch (1.27 cm) OD, 10-inch (25.4 cm) length, 0.35 in (0.89 cm wall thickness) was used as the reactor and was filled with 4 cc of fluorinated Cr₂O₃ catalyst (Newport Cr). A flow of CCl₃CH₂CHCl₂ (R-240fa) at 0.21 ml/hr was vaporized with anhydrous HF at 1:20 mol ratio at a temperature of 150° C. and fed over the catalyst bed at various temperatures. The reaction was carried out at temperatures ranging from 250° C. to 350° C. Part of the reactor effluent was passed through a series of valves and analyzed by Agilent® 7890B GC/5977 MS and a Restek® PC2618 5% Krytox® CBK-D/60/80 6-meter×2 mm ID ⅛″ OD packed column purged with helium at 20 sccm. Samples were taken in 1.5 hours intervals. Compositions comprising HFO-1234ze(Z) were obtained, as shown in Table 5, with the amounts of components being expressed as mole percent.

TABLE 5
Example 2

| | Unknowns | E1234ze | 245fa + Z-1234ze | 1223xf |
|---|---|---|---|---|
| Inj. | % | % | % | % |
| 1 | 0.17 | 3.66 | 31.08 | 0.03 |
| 2 | 0.14 | 3.91 | 36.56 | 0.03 |
| 3 | 0.15 | 4.49 | 45.14 | 0.02 |
| 4 | 0.15 | 4.81 | 49.37 | 0.02 |
| 5 | 0.36 | 4.48 | 44.92 | 0.03 |
| 6 | 0.17 | 4.95 | 50.13 | 0.02 |
| 7 | 0.15 | 4.45 | 44.34 | 0.02 |
| 8 | 0.16 | 7.59 | 34.40 | 0.02 |
| 9 | 0.12 | 6.87 | 30.34 | 0.02 |
| 10 | 0.12 | 7.25 | 33.03 | 0.02 |
| 11 | 0.33 | 7.17 | 32.56 | 0.02 |
| 12 | 0.15 | 6.51 | 29.65 | 0.02 |
| 13 | 0.20 | 9.35 | 20.18 | 0.03 |
| 14 | 0.15 | 9.59 | 20.01 | 0.03 |
| 15 | 0.27 | 9.52 | 20.13 | 0.04 |
| 16 | 0.16 | 10.03 | 21.32 | 0.03 |
| 17 | 0.17 | 10.63 | 22.95 | 0.03 |
| 18 | 0.14 | 10.79 | 23.32 | 0.03 |
| 19 | 0.19 | 14.76 | 16.03 | 0.04 |
| 20 | 0.15 | 13.31 | 15.43 | 0.05 |
| 21 | 0.23 | 17.29 | 19.31 | 0.04 |
| 22 | 0.16 | 13.56 | 14.82 | 0.05 |
| 23 | 0.22 | 15.83 | 17.19 | 0.04 |
| 24 | 0.20 | 16.24 | 10.40 | 0.09 |
| 25 | 0.41 | 19.02 | 12.35 | 0.09 |
| 26 | 0.21 | 16.62 | 10.31 | 0.09 |
| 27 | 0.21 | 19.01 | 12.82 | 0.08 |
| 28 | 0.19 | 16.99 | 10.50 | 0.09 |

| | E-1233zd | Z-1233zd | 244fa | 243fa | 243fb | Temp |
|---|---|---|---|---|---|---|
| Inj. | % | % | % | % | % | ° C. |
| 1 | 57.31 | 5.60 | 1.81 | 0.00 | 0.34 | 250 |
| 2 | 52.07 | 4.98 | 1.98 | 0.00 | 0.33 | 250 |
| 3 | 44.00 | 4.30 | 1.67 | 0.00 | 0.24 | 250 |
| 4 | 39.97 | 3.94 | 1.54 | 0.00 | 0.21 | 250 |
| 5 | 43.98 | 4.28 | 1.70 | 0.00 | 0.25 | 250 |
| 6 | 39.21 | 3.87 | 1.47 | 0.00 | 0.19 | 250 |
| 7 | 44.73 | 4.35 | 1.71 | 0.00 | 0.25 | 250 |
| 8 | 50.85 | 5.40 | 1.43 | 0.00 | 0.15 | 275 |
| 9 | 55.12 | 5.82 | 1.53 | 0.00 | 0.18 | 275 |
| 10 | 52.43 | 5.51 | 1.47 | 0.00 | 0.16 | 275 |
| 11 | 52.64 | 5.59 | 1.51 | 0.00 | 0.18 | 275 |
| 12 | 56.09 | 5.86 | 1.54 | 0.00 | 0.19 | 275 |
| 13 | 61.69 | 7.03 | 1.39 | 0.02 | 0.13 | 300 |
| 14 | 61.72 | 6.99 | 1.36 | 0.02 | 0.13 | 300 |
| 15 | 61.54 | 7.03 | 1.32 | 0.02 | 0.13 | 300 |
| 16 | 60.12 | 6.87 | 1.33 | 0.02 | 0.12 | 300 |
| 17 | 58.11 | 6.68 | 1.30 | 0.02 | 0.12 | 300 |
| 18 | 57.75 | 6.58 | 1.26 | 0.02 | 0.11 | 300 |
| 19 | 60.42 | 7.32 | 1.11 | 0.04 | 0.08 | 325 |
| 20 | 62.29 | 7.50 | 1.13 | 0.06 | 0.09 | 325 |
| 21 | 55.21 | 6.74 | 1.10 | 0.04 | 0.06 | 325 |
| 22 | 62.54 | 7.56 | 1.16 | 0.06 | 0.08 | 325 |
| 23 | 58.36 | 7.09 | 1.14 | 0.05 | 0.07 | 325 |
| 24 | 63.78 | 8.07 | 1.02 | 0.13 | 0.06 | 350 |
| 25 | 59.41 | 7.59 | 0.97 | 0.10 | 0.05 | 350 |
| 26 | 63.44 | 8.06 | 1.06 | 0.15 | 0.07 | 350 |
| 27 | 59.21 | 7.57 | 0.95 | 0.11 | 0.05 | 350 |
| 28 | 62.96 | 8.02 | 1.03 | 0.16 | 0.06 | 350 |

### Example 3: HFO-1234ze(Z) was Prepared as Follows

An Inconel® pipe (0.5-inch (1.27 cm) OD, 10-inch (25.4 cm) length, 0.35 in (0.89 cm wall thickness) was used as the reactor and was filled with 8 cc of fluorinated Cr₂O₃ catalyst (Newport Cr). A flow of CF₃CH═CHCl (R-1233zd) was fed at a rate of 0.41 ml/hr via a vaporizer controlled at 80° C., and anhydrous HF fed was 16 sccm. The reaction was carried out at temperatures ranging from 300° C. to 350° C. Part of the reactor effluent was passed through a series of valves and analyzed by Agilent® 7890B GC/5977 MS and a Restek® PC2618 5% Krytox® CBK-D/60/80 6-meter×2 mm ID ⅛″ OD packed column purged with helium at 20 sccm. Samples were taken in 1.5 hours intervals. Compositions comprising HFO-1234ze(Z) were obtained, as shown in Table 6, with the amounts of components being expressed as mole percent.

TABLE 6
Example 3

| | Unknowns | 1234yf | E-1234ze | 236fa + 245fa | Z-1234ze |
|---|---|---|---|---|---|
| Inj. | % | % | % | % | % |
| 2 | 0.06 | 0.51 | 9.46 | 11.20 | 2.27 |
| 3 | 0.07 | 0.21 | 9.00 | 6.84 | 2.21 |
| 4 | 0.07 | 0.31 | 9.32 | 10.26 | 2.27 |
| 5 | 0.07 | 0.30 | 9.60 | 10.96 | 2.39 |
| 6 | 0.07 | 0.31 | 9.90 | 11.58 | 2.48 |
| 7 | 0.07 | 0.69 | 12.07 | 6.65 | 2.92 |
| 8 | 0.07 | 0.61 | 12.01 | 6.56 | 2.91 |
| 9 | 0.07 | 0.54 | 11.90 | 6.44 | 2.90 |
| 10 | 0.06 | 0.49 | 12.15 | 6.70 | 2.98 |
| 11 | 0.01 | 0.45 | 13.08 | 7.31 | 3.23 |
| 12 | 0.07 | 0.87 | 13.62 | 3.78 | 3.41 |
| 13 | 0.07 | 0.77 | 13.77 | 3.83 | 3.47 |
| 14 | 0.06 | 0.66 | 13.68 | 3.74 | 3.47 |
| 15 | 0.05 | 0.60 | 13.91 | 3.79 | 3.54 |

| | 1233xf | E-1233zd | Z-1233zd | 243fa | |
|---|---|---|---|---|---|
| Inj. | % | % | % | % | Temp ° C. |
| 2 | 1.39 | 67.03 | 7.98 | 0.10 | 300 |
| 3 | 0.86 | 72.28 | 8.34 | 0.18 | 300 |
| 4 | 0.97 | 68.36 | 8.12 | 0.33 | 300 |
| 5 | 0.89 | 67.45 | 8.03 | 0.30 | 300 |
| 6 | 0.87 | 66.56 | 7.94 | 0.30 | 300 |
| 7 | 1.93 | 66.48 | 8.36 | 0.83 | 325 |
| 8 | 1.71 | 66.96 | 8.40 | 0.78 | 325 |
| 9 | 1.57 | 67.35 | 8.50 | 0.74 | 325 |
| 10 | 1.39 | 67.09 | 8.47 | 0.66 | 325 |
| 11 | 1.21 | 65.80 | 8.35 | 0.55 | 325 |
| 12 | 2.38 | 65.54 | 8.80 | 1.41 | 350 |
| 13 | 2.09 | 65.82 | 8.84 | 1.25 | 350 |
| 14 | 1.80 | 66.45 | 8.97 | 1.08 | 350 |
| 15 | 1.60 | 66.48 | 9.01 | 0.95 | 350 |

### Example 4: HFO-1234ze(Z) is Prepared from HFO-1234ze(E) as Follows

An Inconel® pipe (0.5-inch (1.27 cm) OD, 10-inch (25.4 cm) length, 0.35-in (0.89 cm wall thickness) is used as the reactor and is filled with 8 cc of fluorinated Cr₂O₃ catalyst or fluorinated Al₂O₃ catalyst. A flow of HFO-1234ze(E) is fed to the reactor. The reaction is carried out at temperatures ranging from 50° C. to 450° C. Part of the reactor effluent is passed through a series of valves and analyzed by Agilent® 7890B GC/5977 MS and a Restek® PC2618 5% Krytox® CBK-D/60/80 6-meter×2 mm ID ⅛″ OD packed column purged with helium at 20 sccm. The conversion of E-1234ze to Z-1234ze is between 2 to 70%. This isomerization process can also be done in the presence of an oxygen containing gas.

### Example 5

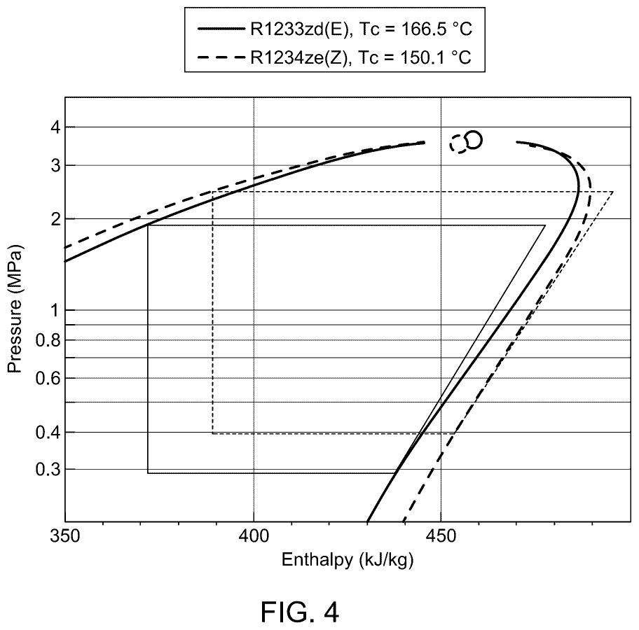

Performance of compositions according to the present invention which comprise R-1234zeZ (neat) in a high temperature heat pump cycle was evaluated as follows in comparison to R-1233zdE as the incumbent fluid. As shown in FIG. 4, in a high temperature heat pump cycle with an 80° C. temperature lift to a 130° C. condensing temperature, R-1233zdE cannot achieve qualitative cycle performance compared to compositions according to the present invention which comprise R-1234zeZ, due to wet compression at these cycle conditions. As a result, the centrifugal compressor's efficiency has to be reduced from 85% to 70% to force the compression path of R-1234zeZ to avoid the 2-phase region. The R-1234zeZ compositions according to the present invention are thus expected to outperform the incumbent fluid R-1233zdE in whatever system is being retrofitted. Also, the R1234zeZ compositions according to the present invention have greater capacity with only a small reduction in efficiency compared to incumbent fluid R-1233zdE, as shown below:

### Conditions:

T_condenser=130.0° C.
T_evaporator=50.0° C.
compressor efficiency=0.7
Average Heat Exchanger Temperature Set Points
100% of superheat is included in refrigeration effect
Vapor Molar Quality Entering Evaporator: q_4
cooling load=3.517 kW
compressor displacement=0.1 (m\hat{ }3/min)

| Fluid | CAP_h (kJ/m\hat{ }3) | COP_h |
|---|---|---|
| R1233zd(E) | 1792.5 | 2.391 |
| R1234ze(Z) | 2191 | 2.265 |

### Performance Examples

Refrigerant performance has been determined for compositions of the present invention in flooded evaporator heating and/or cooling systems. In the result tables for all the examples, the cycle performance metric and fluid property ranges, are calculated from thermophysical properties and fluid phase equilibria of the compositions of the present invention in a vapor compression cycle with a flooded evaporator heat exchanger at the conditions specified. All comparisons for HTHP application were performed at a relatively small centrifugal compressor isentropic efficiency of 70% to directly compare the presently discovered compositions against the HTHP incumbent fluid, R-1233zdE, in the same conditions set without wet compression for the incumbent.

### Example 6: R-1234zeZ/R-1336mzzE/R-1336mzzZ

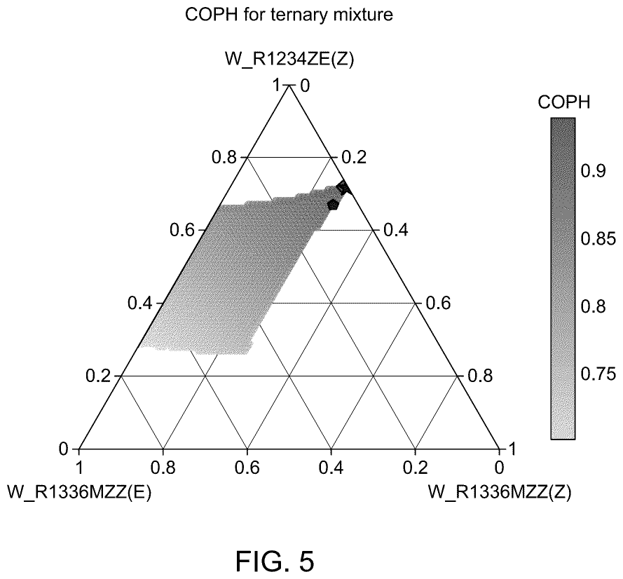

Compositions comprising R-1234zeZ, R-1336mzzE and R-1336mzzZ were explored in a flooded evaporator system for a HTHP, where the liquid level in the evaporator is volumetrically greater than 50%, within class 1 flammability classification, GWP <12 and <1K condenser glide constraints while maintaining 10% of the volumetric heating capacity (CAP_h) of that of R-1233zdE. The working cycle conditions are given in Table 7 and the performance metric and composition property ranges are summarized in Table 8. Table 9 tabulates several compositions about the compositions of optimal heating efficiency and capacity. FIG. 5 provides a graphical representation of the relative COP for heating relative to that of incumbent fluid R-1233zdE, given by COP_h/COP_h_ref, where COP_h_ref is the COP for heating of R-1233zdE.

TABLE 7
HTHP conditions

| | |
|---|---|
| T_condensor (degC.) | 130 |
| T_evaporator (degC.) | 50 |
| superheat (K) | 0 |
| subcooling (K) | 0 |

TABLE 8
HTHP condition, cycle metric performance and fluid property
ranges for R-1234zeZ/R-1336mzzE/R-1336mzzZ in Example 6.

| | MAXIMA | MINIMA |
|---|---|---|
| w_R1234ze(Z) (nominal weight fraction) | 0.720 | 0.270 |
| w_R1336mzz(E) (nominal weight fraction) | 0.706 | 0.005 |
| w_R1336mzz(Z) (nominal weight fraction) | 0.279 | 0.005 |
| R1234ze(Z)_YW_evap | 0.829 | 0.269 |
| R1336mzz(E)_YW_evap | 0.724 | 0.007 |
| R1336mzz(Z)_YW_evap | 0.164 | 0.003 |
| T_c (degC.) | 153 | 137 |
| P_c (MPa) | 3.47 | 3.01 |
| T_discharge (degC.) | 136 | 131 |
| P_evap (MPa) | 0.42 | 0.34 |
| P_cond (MPa) | 2.65 | 2.28 |
| compression ratio | 6.68 | 6.31 |
| condenser glide (K) | 1.00 | 0.06 |
| GWP AR5 | 12 | 1 |
| P_AAD_at_T_cond (%) | 3.50 | 0.14 |
| N_Pr (evap) | 3.99 | 3.45 |
| HTC_flooded (W/m\hat{ }2-K) | 1925 | 1656 |
| COP_h(% dev from R1233ZDE.FLD) | −6.0 | −30.0 |
| CAP_h(% dev from R1233ZDE.FLD) | 10.0 | −10.0 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 9
HTHP condition, cycle metric performance and fluid property
ranges for R-1234zeZ/R-1336mzzE/R-1336mzzZ in Example 6.

| | | | | | CAP_h(% | COP_h(% |
|---|---|---|---|---|---|---|
| wt-% | wt-% | wt-% | GWP | Glide | dev from | dev from |
| R1234ze(Z) | R1336mzz(E) | R1336mzz(Z) | AR5 | (K) | R-1233ZDE) | R-1233ZDE) |
| 40 | 55 | 5 | 9 | 0.22 | −9.98 | −17.95 |
| 30 | 53 | 17 | 9 | 0.62 | −8.16 | −24.47 |
| 30 | 60 | 10 | 10 | 0.36 | −8.36 | −26.52 |
| 31 | 63 | 6 | 11 | 0.23 | −7.96 | −27.22 |
| 29.85 | 69.65 | 0.5 | 11 | 0.06 | −9.15 | −29.46 |
| 35 | 62 | 3 | 10 | 0.14 | −5.69 | −26.22 |
| 38 | 59 | 3 | 10 | 0.15 | −3.93 | −24.87 |
| 39.8 | 59.7 | 0.5 | 10 | 0.08 | −2.99 | −24.78 |
| 47 | 32 | 21 | 6 | 0.82 | 0.47 | −16.24 |
| 48 | 42 | 10 | 7 | 0.40 | 1.41 | −18.84 |
| 51 | 27 | 22 | 5 | 0.86 | 2.21 | −14.47 |
| 51.74 | 47.76 | 0.5 | 8 | 0.08 | 3.37 | −19.90 |
| 55 | 26 | 19 | 5 | 0.74 | 4.18 | −13.85 |
| 57 | 22 | 21 | 5 | 0.82 | 4.88 | −12.60 |
| 60 | 32 | 8 | 6 | 0.33 | 7.00 | −14.99 |
| 62 | 24 | 14 | 5 | 0.55 | 7.49 | −12.77 |
| 63 | 16 | 21 | 4 | 0.80 | 7.31 | −10.58 |
| 67 | 20 | 13 | 4 | 0.50 | 9.58 | −11.39 |
| 71 | 3 | 26 | 2 | 0.94 | 9.73 | −6.70 |
| 72 | 1 | 27 | 1 | 0.97 | 9.95 | −6.14 |

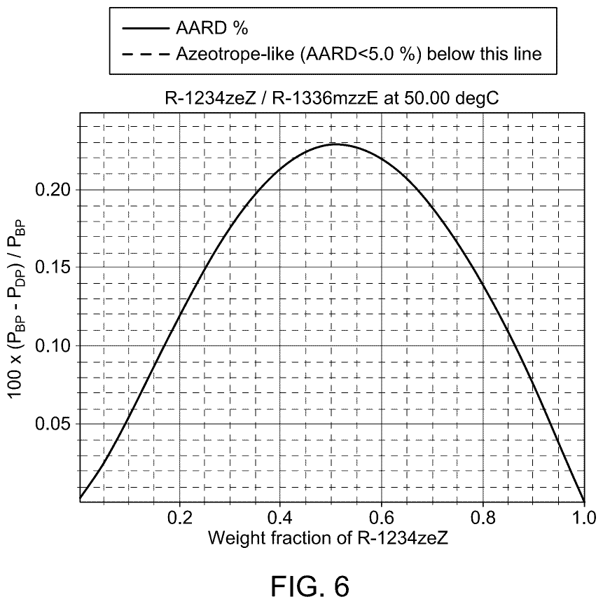

It was found that the addition of R-1336mzzZ both increases the mixture critical temperature and decreases the compressor discharge temperature allowing for greater condensing temperature range, while maintaining about the same COP for heating as neat R-1234zeZ. The addition of R-1336mzzE helps avoid wet compression, lowers the discharge temperature, which preserves the mechanical integrity of the compressor. Adding R-1336mzzE also lowers the condenser glide, lowers the heat of combustion, and raises the LFL of the blend. The entire composition range of the R-1336mzzE/R-1234zeZ binary system is considered azeotrope-like. FIG. 6 demonstrates that its maximum deviation from bubble point to dew point pressure at the evaporator temperature is 0.23% at about 50 wt-% R-1234zeZ. The composition of 72 wt-% R-1234zeZ and 28 wt-% R-1336mzzZ avoids wet compression and has a heating capacity and efficiency of 9.8 and −5.9% of that of R-1233zdE. The composition of 66 wt-% R-1234zeZ and 34 wt-% of R-1336zeE results in 9.9 and −15% of the heating capacity and efficiency of that of R-1233zdE.

While the max heating capacity is toward pure R-1234zeZ, adding R-1336mzzZ and R-1336mzzE increases the efficiency such that R-1234zeZ/R-1336mzzE/R-1336mzzZ at 71.6/0.5/27.9 wt. % represents the maximum COP for heating maintaining a low glide of <1 K. At this maximum efficiency, the volumetric heating capacity is 9.7% greater than incumbent R-1223zdE, while only having an efficiency of 6% less than R-1233zdE, at the same cycle conditions.

### Example 7: R-1234zeZ/R-1336mzzE/R-245fa

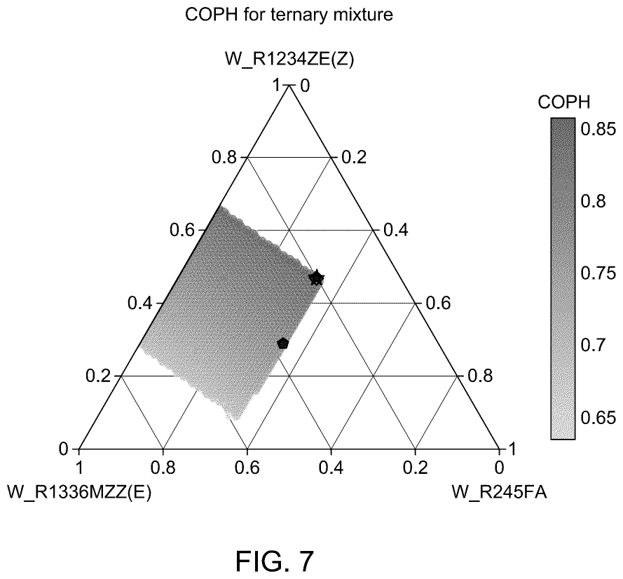

Compositions comprising R-1234zeZ, R-1336mzzE and R-245fa were explored in a flooded evaporator system for a HTHP, where the liquid level in the evaporator is volumetrically greater than 50%, within class 1 flammability classification, GWP <300 and <1K condenser glide constraints while maintaining 10% of the volumetric heating capacity (CAP_h) of that of R-1233zdE. The working cycle conditions are given in Table 7 and the performance metric and composition property ranges are summarized in Table 10. Table 11 tabulates several compositions about the compositions of optimal heating efficiency and capacity. FIG. 7 provides a graphical representation of the relative COP for heating relative to that of incumbent fluid R-1233zdE, given by COP_h/COP_h_ref, where COP_h_ref is the COP for heating of R-1233zdE.

TABLE 10
HTHP condition, cycle metric performance and fluid property
ranges for R-1234zeZ/R-1336mzzE/R-245fa in Example 7.

| | MAXIMA | MINIMA |
|---|---|---|
| w_R1234ze(Z) (nominal weight fraction) | 0.657 | 0.090 |
| w_R1336mzz(E) (nominal weight | 0.706 | 0.200 |
| fraction) | | |
| w_R245fa (nominal weight fraction) | 0.340 | 0.005 |
| R1234ze(Z)_YW_evap | 0.635 | 0.085 |
| R1336mzz(E)_YW_evap | 0.722 | 0.225 |
| R245fa_YW_evap | 0.318 | 0.005 |
| T_c (degC.) | 163 | 137 |
| P_c (MPa) | 4.74 | 3.01 |
| T_discharge (degC.) | 137 | 124 |
| P_evap (MPa) | 0.42 | 0.40 |
| P_cond (MPa) | 2.68 | 2.53 |
| compression ratio | 6.44 | 6.30 |
| condenser glide (K) | 0.09 | 0.05 |
| GWP AR5 | 300 | 10 |
| P_AAD_at_T_cond (%) | 0.18 | 0.09 |
| N_Pr (evap) | 4.21 | 3.45 |
| HTC_flooded (W/m\hat{ }2-K) | 1927 | 1648 |
| COP_h(% dev from R1233ZDE.FLD) | −14.2 | −36.6 |
| CAP_h(% dev from R1233ZDE.FLD) | 10.0 | −10.0 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 11
HTHP condition, cycle metric performance and fluid property
ranges for R-1234zeZ/R-1336mzzE/R-245fa in Example 7.

| | | | | | CAP_h(% | COP_h(% |
|---|---|---|---|---|---|---|
| wt-% | wt-% | wt-% | GWP | Glide | dev from | dev from |
| R1234ze(Z) | R1336mzz(E) | R245fa | AR5 | (K) | R-1233ZDE) | R-1233ZDE) |
| 41 | 43 | 16 | 145 | 0.07 | −2.4 | −16.6 |
| 16 | 59 | 25 | 224 | 0.06 | −8.5 | −29.2 |
| 18 | 64 | 18 | 165 | 0.05 | −9.9 | −30.4 |
| 24 | 48 | 28 | 248 | 0.07 | −2.6 | −24.5 |
| 23 | 65 | 12 | 114 | 0.05 | −9.0 | −29.6 |
| 26 | 61 | 13 | 122 | 0.05 | −6.6 | −27.9 |
| 30 | 44 | 26 | 230 | 0.08 | 0.1 | −22.5 |
| 31 | 52 | 17 | 154 | 0.07 | −2.3 | −24.4 |
| 33 | 43 | 24 | 213 | 0.08 | 1.0 | −21.7 |
| 34 | 54 | 12 | 112 | 0.06 | −2.2 | −24.5 |
| 36 | 45 | 19 | 171 | 0.07 | 1.1 | −21.8 |
| 38 | 38 | 24 | 212 | 0.08 | 3.6 | −19.7 |
| 39 | 56 | 5 | 52 | 0.06 | −1.8 | −24.1 |
| 41 | 54 | 5 | 52 | 0.07 | −0.7 | −23.2 |
| 43 | 50 | 7 | 68 | 0.07 | 1.0 | −21.9 |
| 45 | 44 | 11 | 102 | 0.07 | 3.3 | −20.2 |
| 46 | 27 | 27 | 236 | 0.07 | 8.1 | −16.0 |
| 47 | 21 | 32 | 278 | 0.07 | 9.7 | −14.5 |
| 50.745 | 48.755 | 0.5 | 13 | 0.07 | 3.0 | −20.3 |
| 53 | 44 | 3 | 33 | 0.07 | 4.9 | −18.9 |
| 55 | 32 | 13 | 117 | 0.07 | 8.5 | −16.0 |
| 59 | 35 | 6 | 58 | 0.07 | 8.4 | −16.1 |
| 9 | 60 | 31 | 276 | 0.06 | 3.6 | −36.6 |

R-1234zeZ, R-1336mzzE and R-245fa at 47, 20 and 33 wt. % represents the maximum COP for heating maintaining a low glide of <1 K. At this maximum efficiency, the volumetric heating capacity is 10% greater than incumbent R-1223zdE.

R-245fa contributes to elevating the mixture critical temperature, increasing the range of applicable heating temperatures. It also maintains the heating COP to remain comparable to incumbent fluid R-1233zdE. Addition of R-245fa also helps to keep the glide very low. The entire composition range of Example 7 is azeotrope-like.

### Example 8: R-1234zeZ/R-1336mzzE/Isobutane

Compositions comprising R-1234zeZ, R-1336mzzE and Isobutane were explored in a flooded evaporator system for a HTHP, where the liquid level in the evaporator is volumetrically greater than 50%, within 2 L flammability classification, GWP <12 and <1K condenser glide constraints while maintaining 10% of the volumetric heating capacity (CAP_h) of that of R-1233zdE. The working cycle conditions are given in Table 7 and the performance metric and composition property ranges are summarized in Table 12. Table 13 tabulates several compositions about the compositions of optimal heating efficiency and capacity.

TABLE 12
HTHP condition, cycle metric performance and fluid property
ranges for R-1234zeZ/R-1336mzzE/Isobutane in Example 8.

| | MAXIMA | MINIMA |
|---|---|---|
| w_Isobutane (nominal weight fraction) | 0.100 | 0.005 |
| w_R1234ze(Z) (nominal weight fraction) | 0.840 | 0.299 |
| w_R1336mzz(E) (nominal weight fraction) | 0.697 | 0.060 |
| Isobutane_YW_evap | 0.243 | 0.016 |
| R1234ze(Z)_YW_evap | 0.699 | 0.279 |
| R1336mzz(E)_YW_evap | 0.705 | 0.061 |
| T_c (degC.) | 142 | 130 |
| P_c (MPa) | 3.53 | 3.05 |
| T_discharge (degC.) | 140 | 134 |
| P_evap (MPa) | 0.58 | 0.42 |
| P_cond (MPa) | 3.48 | 2.64 |
| compression ratio | 6.40 | 5.98 |
| condenser glide (K) | 1.00 | 0.42 |
| GWP AR5 | 11 | 2 |
| P_AAD_at_T_cond (%) | 4.38 | 0.42 |
| N_Pr (evap) | 3.71 | 3.13 |
| HTC_flooded (W/m\hat{ }2-K) | 2360 | 1923 |
| COP_h(% dev from R1233ZDE.FLD) | −17.7 | −48.2 |
| CAP_h(% dev from R1233ZDE.FLD) | 9.7 | −9.9 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 13
HTHP condition, cycle metric performance and fluid property
ranges for R-1234zeZ/R-1336mzzE/Isobutane in Example 8.

| | | | | | CAP_h(% | COP_h(% |
|---|---|---|---|---|---|---|
| wt-% | wt-% | wt-% | GWP | Glide | dev from | dev from |
| Isobutane | R1234ze(Z) | R1336zz(E) | AR5 | (K) | R-1233ZDE) | R-1233ZDE) |
| 0.5 | 29.85 | 69.65 | 11 | 3.1 | −32.0 | −9.4 |
| 0.5 | 31.84 | 67.66 | 11 | 3.1 | −30.9 | −8.0 |
| 0.5 | 34.825 | 64.675 | 11 | 3.1 | −29.4 | −5.9 |
| 0.5 | 37.81 | 61.69 | 10 | 3.1 | −28.0 | −4.0 |
| 0.5 | 40.795 | 58.705 | 10 | 3.1 | −26.6 | −2.2 |
| 0.5 | 43.78 | 55.72 | 9 | 3.2 | −25.3 | −0.4 |
| 0.5 | 46.765 | 52.735 | 9 | 3.2 | −24.0 | 1.3 |
| 0.5 | 47.76 | 51.74 | 9 | 3.2 | −23.6 | 1.8 |
| 0.5 | 49.75 | 49.75 | 8 | 3.2 | −22.8 | 2.9 |
| 0.5 | 52.735 | 46.765 | 8 | 3.2 | −21.7 | 4.4 |
| 0.5 | 55.72 | 43.78 | 8 | 3.2 | −20.5 | 5.9 |
| 0.5 | 58.705 | 40.795 | 7 | 3.3 | −19.4 | 7.4 |
| 0.5 | 61.69 | 37.81 | 7 | 3.3 | −18.4 | 8.8 |
| 5 | 55 | 40 | 7 | 3.4 | −45.3 | −9.9 |
| 6 | 64 | 30 | 6 | 3.4 | −43.9 | −5.6 |
| 10 | 80 | 10 | 3 | 3.5 | −42.2 | 1.7 |
| 10 | 83 | 7 | 2 | 3.5 | −38.4 | 7.8 |
| 9 | 75 | 16 | 4 | 3.5 | −48.2 | −9.1 |
| 9 | 79 | 12 | 3 | 3.5 | −40.4 | 3.8 |
| 9 | 80 | 11 | 3 | 3.5 | −39.3 | 5.6 |
| 8 | 73 | 19 | 4 | 3.5 | −44.9 | −4.5 |
| 8 | 76 | 16 | 4 | 3.5 | −40.5 | 2.6 |

R-1234zeZ, R-1336mzzE and Isobutane at 63.7, 35.8 and 0.5 wt. % represents the maximum COP and CAP for heating maintaining a low glide of <1 K. At this maximum efficiency, the volumetric heating capacity is 10% greater than incumbent R-1223zdE, while the efficiency is only-18% of the efficiency of R-1233zdE.

Isobutane contributes to increasing the heat transfer coefficient for pool boiling in the evaporator.

### Example 9: R-1234zeZ/R-1336mzzE/R-227ea

Compositions comprising R-1234zeZ, R-1336mzzE and R-227ea were explored in a flooded evaporator system for a HTHP, where the liquid level in the evaporator is volumetrically greater than 50%, within 1 flammability classification, GWP <277 and <1K condenser glide constraints while maintaining 10% of the volumetric heating capacity (CAP_h) of that of R-1233zdE. The working cycle conditions are given in Table 7 and the performance metric and composition property ranges are summarized in Table 14. Table 15 tabulates several compositions about the compositions of optimal heating efficiency and capacity.

TABLE 14
HTHP condition, cycle metric performance and fluid property
ranges for R-1234zeZ/R-1336mzzE/R-227ea in Example 9.

| | MAXIMA | MINIMA |
|---|---|---|
| w_R1234ze(Z) (nominal weight fraction) | 0.680 | 0.299 |
| w_R1336mzz(E) (nominal weight fraction) | 0.697 | 0.240 |
| w_R245fa (nominal weight fraction) | 0.080 | 0.005 |
| R1234ze(Z)_YW_evap | 0.633 | 0.281 |
| R1336mzz(E)_YW_evap | 0.709 | 0.240 |
| R245fa_YW_evap | 0.144 | 0.010 |
| T_c (degC.) | 149 | 135 |
| P_c (MPa) | 3.88 | 3.02 |
| T_discharge (degC.) | 135 | 132 |
| P_evap (MPa) | 0.45 | 0.40 |
| P_cond (MPa) | 2.83 | 2.55 |
| compression ratio | 6.35 | 6.30 |
| condenser glide (K) | 0.81 | 0.10 |
| GWP AR5 | 277 | 23 |
| P_AAD_at_T_cond (%) | 0.96 | 0.15 |
| N_Pr (evap) | 3.73 | 3.41 |
| HTC_flooded (W/m\hat{ }2-K) | 1963 | 1897 |
| COP_h(% dev from R1233ZDE.FLD) | −15.4 | −34.2 |
| CAP_h(% dev from R1233ZDE.FLD) | 9.9 | −10.0 |

YW_evap: weight fraction-based composition of the vapor leaving the flooded evaporator.
N_Pr (evap): Prandtl Number in the conditions of the evaporator
HTC_flooded (W/m\hat{ }2-K): Cooper (1984) correlation for heat transfer coefficient for pool boiling in the evaporator

TABLE 15
HTHP condition, cycle metric performance and fluid property
ranges for R-1234zeZ/R-1336mzzE/R-227ea in Example 9.

| | | | | | CAP_h(% | COP_h(% |
|---|---|---|---|---|---|---|
| wt-% | wt-% | wt-% | GWP | Glide | dev from | dev from |
| R1234ze(Z) | R1336zz(E) | R245fa | AR5 | (K) | R-1233ZDE) | R-1233ZDE) |
| 43 | 54 | 3 | 110 | 0.4 | −21.6 | −8.5 |
| 32 | 66 | 2 | 78 | 0.2 | −30.3 | −8.7 |
| 34 | 63 | 3 | 111 | 0.3 | −30.2 | −7.8 |
| 33 | 62 | 5 | 178 | 0.5 | −32.6 | −9.6 |
| 38 | 58 | 4 | 144 | 0.4 | −29.0 | −5.6 |
| 37 | 57 | 6 | 210 | 0.5 | −31.3 | −7.2 |
| 44 | 54 | 2 | 76 | 0.3 | −24.6 | −1.2 |
| 40 | 53 | 7 | 243 | 0.6 | −30.5 | −5.6 |
| 42 | 51 | 7 | 243 | 0.6 | −29.5 | −4.3 |
| 47 | 49 | 4 | 142 | 0.5 | −24.8 | −0.1 |
| 48 | 47 | 5 | 176 | 0.5 | −25.1 | 0.2 |
| 51 | 46 | 3 | 108 | 0.4 | −22.4 | 2.4 |
| 53.73 | 45.77 | 0.5 | 25 | 0.1 | −19.6 | 4.3 |
| 55 | 44 | 1 | 41 | 0.2 | −19.5 | 4.8 |
| 56.715 | 42.785 | 0.5 | 24 | 0.1 | −18.5 | 5.7 |
| 58 | 41 | 1 | 41 | 0.2 | −18.4 | 6.3 |
| 58 | 35 | 7 | 241 | 0.7 | −22.3 | 5.2 |
| 61 | 38 | 1 | 40 | 0.2 | −17.3 | 7.6 |
| 62 | 33 | 5 | 173 | 0.6 | −19.5 | 7.6 |
| 64 | 34 | 2 | 73 | 0.3 | −16.9 | 8.9 |
| 65 | 28 | 7 | 240 | 0.7 | −19.6 | 8.7 |
| 68 | 24 | 8 | 273 | 0.8 | −19.1 | 9.9 |

R-1234zeZ, R-1336mzzE and R-227ea at 65.7, 33.8 and 0.5 wt. % represents the maximum COP and CAP for heating maintaining a low glide of <1 K. At this maximum efficiency, the volumetric heating capacity is 10% greater than incumbent R-1223zdE, while the efficiency is only-15% of the efficiency of R-1233zdE.

R-227ea contributes to increasing the heat transfer coefficient for pool boiling in the evaporator.

### Example 10: R-1234zeZ/R-1336mzzE/R-1336mzzZ/R-134

Compositions comprising R-1234zeZ, R-1336mzzE, R-1336mzzZ and R-134 were explored in a flooded evaporator system for a HTHP, where the liquid level in the evaporator is volumetrically greater than 50%, within 1 flammability classification, GWP <295 and <1K condenser glide constraints while maintaining 10% of the volumetric heating capacity (CAP_h) of that of R-1233zdE. The working cycle conditions are given in Table 7 and the performance metric and composition property ranges are summarized in Table 16. Table 17 tabulates several compositions about the compositions of optimal heating efficiency and capacity.

TABLE 16
HTHP condition, cycle metric performance and fluid property ranges
for R-1234zeZ/R-1336mzzE/R-1336mzzZ/R-134 in Example 10.

| | MAXIMA | MINIMA |
|---|---|---|
| w_R1234ze(Z) (nominal weight fraction) | 0.637 | 0.005 |
| w_R1336mzz(E) (nominal weight fraction) | 0.736 | 0.139 |
| w_R1336mzz(Z) (nominal weight fraction) | 0.597 | 0.003 |
| w_R134 (nominal weight fraction) | 0.260 | 0.003 |
| R1234ze(Z)_YW_cond | 0.641 | 0.004 |
| R1336mzz(E)_YW_cond | 0.734 | 0.125 |
| R1336mzz(Z)_YW_cond | 0.257 | 0.001 |
| R134_YW_cond | 0.608 | 0.009 |
| T_c (degC.) | 146 | 130 |
| P_c (MPa) | 4.24 | 3.02 |
| T_discharge (degC.) | 145 | 133 |
| P_evap (MPa) | 0.65 | 0.39 |
| P_cond (MPa) | 4.22 | 2.50 |
| compression ratio | 7.20 | 6.32 |
| condenser glide (K) | 1.00 | 0.00 |
| GWP AR5 | 295 | 9 |
| P_AAD_at_T_cond (%) | 25.59 | 0.21 |
| N_Pr (evap) | 4.06 | 3.21 |
| HTC_flooded (W/m\hat{ }2-K) | 2281 | 1701 |
| COP_h(% dev from R1233ZDE.FLD) | −13.1 | −53.4 |
| CAP_h(% dev from R1233ZDE.FLD) | 10.0 | −10.0 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 17
HTHP condition, cycle metric performance and fluid property ranges
for R-1234zeZ/R-1336mzzE/R-1336mzzZ/R-134 in Example 10.

| | | | | | | CAP_h(% | COP_h(% |
|---|---|---|---|---|---|---|---|
| wt-% | wt-% | wt-% | wt-% | GWP | | dev from | dev from |
| R1234ze(Z) | R1336zz(E) | R1336zz(Z) | R134 | AR5 | Glide (K) | R-1233ZDE) | R-1233ZDE) |
| 27.86 | 57.71 | 13.93 | 0.5 | 15 | 0.828199 | −26.2 | −8.9 |
| 29.85 | 59.7 | 9.95 | 0.5 | 16 | 0.67234 | −26.9 | −7.1 |
| 31.68 | 57.42 | 9.9 | 1 | 21 | 0.973535 | −25.6 | −5.8 |
| 33.83 | 63.68 | 1.99 | 0.5 | 16 | 0.391034 | −27.3 | −5.2 |
| 39.8 | 45.77 | 13.93 | 0.5 | 14 | 0.927424 | −21.3 | −1.3 |
| 39.8 | 57.71 | 1.99 | 0.5 | 15 | 0.424909 | −24.6 | −1.6 |
| 35.64 | 57.42 | 5.94 | 1 | 21 | 0.831396 | −25.7 | −2.6 |
| 45.77 | 51.74 | 1.99 | 0.5 | 14 | 0.455648 | −22.1 | 1.7 |
| 41.58 | 51.48 | 5.94 | 1 | 20 | 0.892349 | −23.1 | 0.9 |
| 51.74 | 39.8 | 7.96 | 0.5 | 13 | 0.729679 | −18.3 | 4.7 |
| 53.73 | 35.82 | 9.95 | 0.5 | 12 | 0.827672 | −17.0 | 5.7 |
| 57.71 | 39.8 | 1.99 | 0.5 | 13 | 0.507702 | −17.6 | 7.6 |
| 61.38 | 37.62 | 0.5 | 0.5 | 12 | 0.462186 | −16.7 | 9.3 |
| 24 | 58 | 6 | 12 | 144 | 0.799015 | −46.4 | −7.2 |
| 22 | 46 | 16 | 16 | 187 | 0.853566 | −46.2 | −1.7 |
| 30 | 40 | 12 | 18 | 209 | 0.896061 | −44.3 | 5.4 |
| 34 | 38 | 10 | 18 | 208 | 0.947052 | −43.5 | 7.2 |
| 16 | 30 | 34 | 20 | 230 | 0.944858 | −46.9 | 1.6 |
| 47.76 | 29.85 | 0.5 | 21.89 | 250 | 0.191851 | −47.7 | 4.9 |
| 12 | 20 | 44 | 24 | 273 | 0.465277 | −49.9 | 0.1 |
| 4 | 14 | 56 | 26 | 295 | 0.788319 | −48.5 | 4.4 |
| 12 | 14 | 48 | 26 | 295 | 0.993132 | −46.1 | 9.8 |

R-1234zeZ, R-1336mzzE, R-1336mzzZ and R-134 at 63.7, 23.9, 11.9 and 0.5 wt. % represents the maximum COP and CAP for heating maintaining a low glide of <1 K. At this maximum efficiency, the volumetric heating capacity is 10% greater than incumbent R-1223zdE, while the efficiency is only-13.1% of the efficiency of R-1233zdE.

Adding R-134 to R-1234zeZ/R-1336mzzE/R-1336mzzZ increases the heat transfer coefficient for pool boiling in the evaporator.

### Example 11: R-1234zeZ/R-1336mzzZ/R-1130(E)

Compositions comprising R-1234zeZ, R-1336mzzZ and R-1130E(E) (t-DCE) were explored in a flooded evaporator system for a chiller, where the liquid level in the evaporator is volumetrically greater than 50%, within class 1 flammability classification, GWP <2 and <1K condenser glide constraints while maintaining 10% of the volumetric cooling capacity (CAP_c) of that of incumbent working fluid R-514A. The working cycle conditions are given in Table 18 and the performance metric and composition property ranges are summarized in Table 19. Table 20 tabulates several compositions about the compositions of optimal heating efficiency and capacity.

TABLE 18
Centrifugal chiller conditions

| | |
|---|---|
| T_condensor (° C.) | 37.78 |
| T_evaporator (° C.) | 4.44 |
| superheat (K) | 0 |
| subcooling (K) | 0 |

TABLE 19
Chiller conditions, cycle metric performance and fluid property
ranges for R-1234zeZ/R-1336mzzZ/R-1130(E) in Example 11.

| | MAXIMA | MINIMA |
|---|---|---|
| w_R1234ze(Z) (nominal weight fraction) | 0.030 | 0.005 |
| w_R1336mzz(Z) (nominal weight fraction) | 0.925 | 0.289 |
| w_R1130(E) (nominal weight fraction) | 0.706 | 0.070 |
| R1234ze(Z)_YW_evap | 0.065 | 0.011 |
| R1336mzz(Z)_YW_evap | 0.849 | 0.674 |
| R1130(E)_YW_evap | 0.302 | 0.139 |
| T_c (degC.) | 181 | 172 |
| P_c (MPa) | 3.66 | 3.16 |
| T_discharge (degC.) | 49 | 44 |
| P_evap (MPa) | 0.04 | 0.04 |
| P_cond (MPa) | 0.15 | 0.14 |
| compression ratio | 3.86 | 3.69 |
| condenser glide (K) | 1.00 | 0.22 |
| GWP AR5 | 2 | 1 |
| P_AAD_at_T_cond (%) | 52.13 | 0.33 |
| N_Pr (evap) | 6.40 | 3.71 |
| HTC_flooded (W/m\hat{ }2-K) | 922 | 872 |
| COP_c(% dev from R-514A) | −0.3 | −3.3 |
| CAP_c(% dev from R514A) | 4.1 | −6.4 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 20
Chiller conditions, cycle metric performance and fluid property
ranges for R-1234zeZ/R-1336mzzZ/R-1130E in Example 11.

| | | | | | CAP_c(% | COP_c(% |
|---|---|---|---|---|---|---|
| wt-% | wt-% | wt-% | GWP | Glide | dev from | dev from |
| R1234ze(Z) | R1336mzz(Z) | R1130(E) | AR5 | (K) | R-514A) | R-514A) |
| 0.5 | 92.535 | 6.965 | 2 | 1.0 | −3.3 | −6.4 |
| 0.5 | 30.845 | 68.655 | 1 | 0.8 | −2.6 | −2.2 |
| 0.5 | 32.835 | 66.665 | 1 | 0.7 | −2.2 | −1.6 |
| 0.5 | 87.56 | 11.94 | 2 | 0.5 | −1.9 | −2.4 |
| 1 | 85 | 14 | 2 | 0.6 | −1.7 | −0.6 |
| 0.5 | 37.81 | 61.69 | 1 | 0.6 | −1.5 | −0.6 |
| 2 | 79 | 19 | 2 | 0.7 | −1.4 | 2.4 |
| 0.5 | 84.575 | 14.925 | 2 | 0.3 | −1.3 | −1.0 |
| 0.5 | 40.795 | 58.705 | 1 | 0.5 | −1.2 | −0.2 |
| 0.5 | 41.79 | 57.71 | 1 | 0.5 | −1.1 | −0.1 |
| 1 | 49 | 50 | 1 | 0.6 | −1.0 | 1.3 |
| 2 | 70 | 28 | 1 | 0.8 | −1.0 | 3.2 |
| 1 | 53 | 46 | 1 | 0.6 | −0.9 | 1.4 |
| 1 | 57 | 42 | 1 | 0.6 | −0.8 | 1.5 |
| 1 | 60 | 39 | 1 | 0.5 | −0.7 | 1.5 |
| 1 | 75 | 24 | 2 | 0.4 | −0.6 | 1.5 |
| 1 | 73 | 26 | 1 | 0.4 | −0.6 | 1.6 |
| 0.5 | 78.605 | 20.895 | 2 | 0.2 | −0.5 | 0.4 |
| 0.5 | 60.695 | 38.805 | 1 | 0.3 | −0.4 | 0.7 |
| 0.5 | 73.63 | 25.87 | 1 | 0.2 | −0.3 | 0.8 |

R-1234zeZ/R-1336mzzZ/R-1130E at 0.5/69.7/29.8 wt. % represents the maximum COP for cooling maintaining a low glide of <1 K. At this maximum efficiency, the volumetric cooling capacity is about the same as that for incumbent R-514A, while the efficiency is 1% greater than for R-514A (incumbent fluid), at the same cycle conditions. Additional amounts of R-1234zeZ would increase the cooling capacity further.

### Example 12: R-1234zeZ/R-1336mzzZ/R-1224ydZ

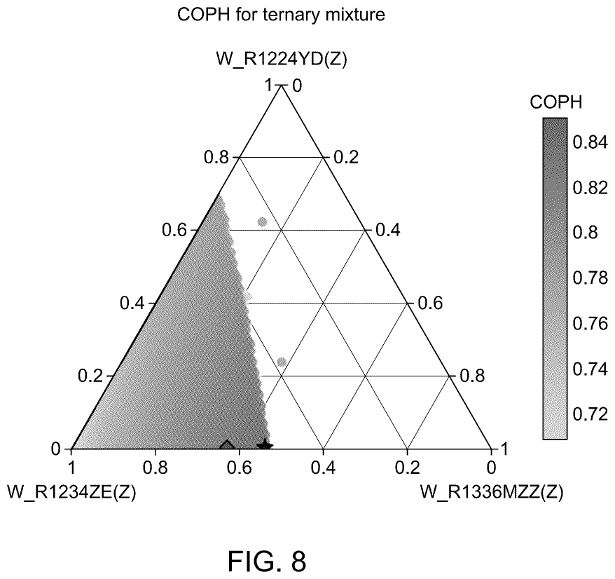

Compositions comprising R-1234zeZ, R-1336mzzZ and R-1224ydZ were explored in a flooded evaporator system for a HTHP delivering heat up to 150° C., where the liquid level in the evaporator is volumetrically greater than 50%, within class 1 flammability classification, GWP <2 and <1K condenser glide constraints while maintaining 10% of the volumetric heating capacity (CAP_h) of that of incumbent working fluid R-1233zdE. The working cycle conditions are given in Table 21 and the performance metric and composition property ranges are summarized in Table 22. Table 23 tabulates several compositions about the compositions of optimal heating efficiency and capacity. FIG. 8 provides a graphical representation of the relative COP for heating relative to that of incumbent fluid R-1233zdE, given by COP_h/COP_h_ref, where COP_h_ref is the COP for heating of R-1233zdE.

TABLE 21
HTHP conditions

| | |
|---|---|
| T_condensor (° C.) | 150 |
| T_evaporator (° C.) | 70 |
| superheat (K) | 0 |
| subcooling (K) | 0 |

TABLE 22
HTHP condition, cycle metric performance and fluid property
ranges for R-1234zeZ/R-1336mzzZ/R-1224ydZ in Example 12.

| | MAXIMA | MINIMA |
|---|---|---|
| w_R1234ze(Z) (nominal weight fraction) | 0.687 | 0.005 |
| w_R1336mzz(Z) (nominal weight | 0.985 | 0.230 |
| fraction) | | |
| w_R1224yd(Z) (nominal weight fraction) | 0.458 | 0.005 |
| R1234ze(Z)_YW_evap | 0.659 | 0.005 |
| R1336mzz(Z)_YW_evap | 0.990 | 0.266 |
| R1224yd(Z)_YW_evap | 0.319 | 0.003 |
| T_c (degC.) | 172 | 150 |
| P_c (MPa) | 4.83 | 3.38 |
| T_discharge (degC.) | 162 | 155 |
| P_evap (MPa) | 0.67 | 0.54 |
| P_cond (MPa) | 3.52 | 3.01 |
| compression ratio | 5.65 | 5.25 |
| condenser glide (K) | 1.00 | 0.00 |
| GWP AR5 | 1 | 1 |
| P_AAD_at_T_cond (%) | 2.57 | 0.01 |
| N_Pr (evap) | 3.50 | 2.96 |
| HTC_flooded (W/m\hat{ }2-K) | 2440 | 2077 |
| COP_h(% dev from R1233ZDE) | −14.9 | −29.2 |
| CAP_h(% dev from R1233ZDE) | 9.2 | −10.0 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 23
HTHP conditions, cycle metric performance and fluid property
ranges for R-1234zeZ/R-1336mzzE/R-1224ydZ in Example 12.

| | | | | | CAP_c(% | COP_c(% |
|---|---|---|---|---|---|---|
| wt-% | wt-% | wt-% | GWP | Glide | dev from | dev from |
| R1224yd(Z) | R1234ze(Z) | R1336mzz(Z) | AR5 | (K) | R-1233zdE) | R-1233zdE) |
| 67 | 31 | 2 | 1 | 0.1 | −21.0 | −10.0 |
| 29 | 44 | 27 | 1 | 0.6 | −17.5 | −10.0 |
| 34 | 44 | 22 | 1 | 0.5 | −18.4 | −9.3 |
| 32 | 46 | 22 | 1 | 0.5 | −18.4 | −8.9 |
| 25 | 49 | 26 | 1 | 0.5 | −17.9 | −8.7 |
| 42 | 47 | 11 | 1 | 0.2 | −20.1 | −8.2 |
| 5 | 56 | 39 | 1 | 0.8 | −16.0 | −8.6 |
| 50.745 | 48.755 | 0.5 | 1 | 0.1 | −22.1 | −7.9 |
| 10 | 58 | 32 | 1 | 0.6 | −17.0 | −7.6 |
| 31 | 56 | 13 | 1 | 0.3 | −20.1 | −7.1 |
| 18 | 60 | 22 | 1 | 0.4 | −18.7 | −6.8 |
| 5 | 64 | 31 | 1 | 0.6 | −17.3 | −6.6 |
| 18 | 64 | 18 | 1 | 0.3 | −19.5 | −6.1 |
| 27 | 65 | 8 | 1 | 0.2 | −21.5 | −6.2 |
| 23 | 68 | 9 | 1 | 0.2 | −21.5 | −5.9 |
| 3 | 73 | 24 | 1 | 0.4 | −18.7 | −5.1 |
| 11 | 75 | 14 | 1 | 0.2 | −20.8 | −5.1 |
| 16 | 78 | 6 | 1 | 0.1 | −22.9 | −5.7 |
| 17 | 82 | 1 | 1 | 0.0 | −25.0 | −6.7 |
| 5 | 88 | 7 | 1 | 0.1 | −23.6 | −5.4 |

R-1234zeZ/R-1336mzzZ/R-1224ydZ at 53.7/45.8/60.5 wt. % represents the maximum COP for heating maintaining a low glide of <1 K. At this maximum efficiency, the volumetric heat capacity is-10% of that of R-1233zdE. A composition of R-1234zeZ, R-1336mzzZ and R-1224ydZ at 37, 21 and 42 wt-% respectively achieves a heating capacity and efficiency of 9.2% and −28% compared to those of R-1233zdE.

### Example 13: R-1234zeZ/R-1234zeE/R-1336mzzE

Compositions comprising R-1234zeZ, R-1234zeE and R-1336mzzE were explored in a flooded evaporator system for a HTHP, where the liquid level in the evaporator is volumetrically greater than 50%, within class 2 L flammability classification, GWP <300 and <1K condenser glide constraints while maintaining 10% of the volumetric heating capacity (CAP_h) of that of R-1233zdE. The working cycle conditions are given in Table 7 and the performance metric and composition property ranges are summarized in Table 24. Table 25 tabulates several compositions about the compositions of optimal heating efficiency and capacity.

TABLE 24
HTHP condition, cycle metric performance and fluid property
ranges for R-1234zeZ/R-1234zeE/R-1336mzzE in Example 13.

| | MAXIMA | MINIMA |
|---|---|---|
| w_R1234ze(E) (nominal weight fraction) | 20 | 4 |
| w_R1234ze(Z) (nominal weight fraction) | 88 | 36 |
| w_R1336mzz(E) (nominal weight fraction) | 60 | 4 |
| R1234ze(E)_YW_evap | 42 | 8 |
| R1234ze(Z)_YW_evap | 83 | 33 |
| R1336mzz(E)_YW_evap | 59 | 3 |
| T_c (degC.) | 146 | 134 |
| P_c (MPa) | 3.7 | 3.2 |
| T_discharge (degC.) | 141 | 135 |
| P_evap (MPa) | 0.55 | 0.42 |
| P_cond (MPa) | 3.3 | 2.7 |
| compression ratio | 6.39 | 6.35 |
| condenser glide (K) | 2.4 | 0.8 |
| GWP AR5 | 10 | 1.6 |
| P_AAD_at_T_cond (%) | 15 | 3.8 |
| N_Pr (evap) | 3.6 | 3.2 |
| HTC_flooded (W/m\hat{ }2-K) | 2157 | 1951 |
| COP_h(% dev from R1233ZDE.FLD) | −30 | −10 |
| CAP_h(% dev from R1233ZDE.FLD) | 28 | −2 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 25
HTHP condition, cycle metric performance and fluid property
ranges for R-1234zeZ/R-1234zeE/R-1336mzzE in Example 13

| | | | | | CAP_h(% | COP_h(% |
|---|---|---|---|---|---|---|
| wt-% | wt-% | wt-% | GWP | Glide | dev from | dev from |
| R1234ze(E) | R1234ze(Z) | R1336mzz(E) | AR5 | (K) | R-1233ZDE) | R-1233ZDE) |
| 16 | 80 | 4 | 1.6 | 2.4 | 28 | −20 |
| 12 | 84 | 4 | 1.6 | 2.3 | 28 | −17 |
| 20 | 76 | 4 | 1.6 | 2.3 | 28 | −24 |
| 16 | 76 | 8 | 2.2 | 2.3 | 27 | −22 |
| 8 | 88 | 4 | 1.6 | 2.0 | 27 | −13 |
| 12 | 80 | 8 | 2.2 | 2.2 | 27 | −18 |
| 8 | 84 | 8 | 2.2 | 1.9 | 25 | −14 |
| 24 | 72 | 4 | 1.6 | 2.0 | 25 | −29 |
| 12 | 76 | 12 | 2.8 | 2.2 | 25 | −19 |
| 16 | 72 | 12 | 2.8 | 2.2 | 25 | −23 |
| 4 | 92 | 4 | 1.6 | 1.3 | 24 | −10 |
| 8 | 80 | 12 | 2.8 | 1.9 | 24 | −15 |
| 4 | 88 | 8 | 2.2 | 1.2 | 23 | −11 |
| 12 | 72 | 16 | 3.4 | 2.1 | 23 | −20 |
| 20 | 68 | 12 | 2.8 | 2.0 | 23 | −27 |
| 16 | 68 | 16 | 3.4 | 2.1 | 23 | −24 |
| 8 | 76 | 16 | 3.4 | 1.8 | 22 | −17 |
| 4 | 84 | 12 | 2.8 | 1.2 | 21 | −12 |
| 12 | 68 | 20 | 4 | 2.0 | 21 | −22 |
| 16 | 64 | 20 | 4 | 2.0 | 20 | −26 |
| 8 | 72 | 20 | 4 | 1.8 | 20 | −18 |
| 20 | 64 | 16 | 3.4 | 1.9 | 20 | −29 |
| 4 | 80 | 16 | 3.4 | 1.2 | 20 | −13 |
| 12 | 64 | 24 | 4.6 | 1.9 | 19 | −23 |
| 4 | 76 | 20 | 4 | 1.2 | 18 | −14 |
| 8 | 68 | 24 | 4.6 | 1.7 | 18 | −19 |
| 16 | 60 | 24 | 4.6 | 1.9 | 18 | −28 |
| 4 | 72 | 24 | 4.6 | 1.1 | 17 | −15 |
| 12 | 60 | 28 | 5.2 | 1.8 | 16 | −25 |
| 8 | 64 | 28 | 5.2 | 1.6 | 16 | −21 |
| 4 | 68 | 28 | 5.2 | 1.1 | 15 | −17 |
| 16 | 56 | 28 | 5.2 | 1.7 | 15 | −29 |
| 8 | 60 | 32 | 5.8 | 1.6 | 14 | −22 |
| 12 | 56 | 32 | 5.8 | 1.7 | 14 | −26 |
| 4 | 64 | 32 | 5.8 | 1.1 | 13 | −18 |
| 8 | 56 | 36 | 6.4 | 1.5 | 12 | −23 |
| 12 | 52 | 36 | 6.4 | 1.6 | 12 | −28 |
| 4 | 60 | 36 | 6.4 | 1.0 | 11 | −19 |
| 8 | 52 | 40 | 7 | 1.4 | 10 | −25 |
| 4 | 56 | 40 | 7 | 1.0 | 9 | −21 |
| 12 | 48 | 40 | 7 | 1.5 | 9 | −30 |
| 8 | 48 | 44 | 7.6 | 1.4 | 8 | −27 |
| 4 | 52 | 44 | 7.6 | 0.9 | 7 | −22 |
| 8 | 44 | 48 | 8.2 | 1.3 | 5 | −28 |
| 4 | 48 | 48 | 8.2 | 0.9 | 5 | −24 |
| 4 | 44 | 52 | 8.8 | 0.9 | 3 | −26 |
| 4 | 40 | 56 | 9.4 | 0.8 | 0 | −27 |
| 4 | 36 | 60 | 10 | 0.8 | −2 | −29 |

R-1234zeZ, R-1234zeE and R-1336mzzE at 92 wt. %, 4 wt. % and 4 wt. %, respectively, represents the maximum COP for heating maintaining a low glide of <2 K. At this maximum efficiency, the volumetric heating capacity is 28% greater than incumbent R-1223zdE, while the efficiency is-17% variation of the efficiency of R-1233zdE.

### Example 14: R-1234zeZ/R-1234zeE/R-134

Compositions comprising R-1234zeZ, R-1234zeE and R-134 were explored in a flooded evaporator system for a HTHP, where the liquid level in the evaporator is volumetrically greater than 50%, within class 1 flammability classification, GWP <300 and <1K condenser glide constraints while maintaining 10% of the volumetric heating capacity (CAP_h) of that of R-1233zdE. The working cycle conditions are given in Table 7 and the performance metric and composition property ranges are summarized in Table 26. Table 27 tabulates several compositions about the compositions of optimal heating efficiency and capacity.

TABLE 26
HTHP condition, cycle metric performance and fluid property
ranges for R-1234zeZ/R-1234zeE/R-134 in Example 14

| | MAXIMA | MINIMA |
|---|---|---|
| w_R1234ze(E) (nominal weight fraction) | 18 | 2 |
| w_R1234ze(Z) (nominal weight fraction) | 94 | 74 |
| w_R134 (nominal weight fraction) | 12 | 2 |
| R1234ze(E)_YW_evap | 41 | 4 |
| R1234ze(Z)_YW_evap | 96 | 74 |
| R134_YW_evap | 12 | 2 |
| T_c (degC.) | 147 | 133 |
| P_c (MPa) | 4 | 3.7 |
| T_discharge (degC.) | 145 | 141 |
| P_evap (MPa) | 0.6 | 0.4 |
| P_cond (MPa) | 3.7 | 2.7 |
| compression ratio | 6.5 | 6.3 |
| condenser glide (K) | 3.6 | 2 |
| GWP AR5 | 23 | 135 |
| P_AAD_at_T_cond (%) | 6 | 1.7 |
| N_Pr (evap) | 3.23 | 3.15 |
| HTC_flooded (W/m\hat{ }2-K) | 2243 | 2016 |
| COP_h(% dev from R1233ZDE.FLD) | −30 | −9 |
| CAP_h(% dev from R1233ZDE.FLD) | 43 | 28 |

YW_evap: weight fraction-based composition of the vapor leaving the flooded evaporator.
N_Pr (evap): Prandtl Number in the conditions of the evaporator.
HTC_flooded (W/m\hat{ }2-K): Cooper (1984) correlation for heat transfer coefficient for pool boiling in the evaporator.

TABLE 27
HTHP condition, cycle metric performance and fluid property
ranges for R-1234zeZ/R-1234zeE/R-134 in Example 14

| | | | | | CAP_h(% | COP_h(% |
|---|---|---|---|---|---|---|
| wt-% | wt-% | wt-% | GWP | Glide | dev from | dev from |
| R1234ze(E) | R1234ze(Z) | R134 | AR5 | (K) | R-1233ZDE) | R-1233ZDE) |
| 2 | 86 | 12 | 135.28 | 3.5 | 43 | −18 |
| 4 | 84 | 12 | 135.28 | 3.3 | 43 | −20 |
| 6 | 82 | 12 | 135.28 | 3.1 | 42 | −22 |
| 2 | 88 | 10 | 112.9 | 3.6 | 42 | −16 |
| 4 | 86 | 10 | 112.9 | 3.5 | 41 | −18 |
| 8 | 80 | 12 | 135.28 | 2.9 | 41 | −24 |
| 6 | 84 | 10 | 112.9 | 3.3 | 41 | −20 |
| 8 | 82 | 10 | 112.9 | 3.1 | 40 | −22 |
| 10 | 78 | 12 | 135.28 | 2.7 | 40 | −26 |
| 10 | 80 | 10 | 112.9 | 2.9 | 39 | −24 |
| 2 | 90 | 8 | 90.52 | 3.6 | 39 | −15 |
| 4 | 88 | 8 | 90.52 | 3.5 | 39 | −16 |
| 6 | 86 | 8 | 90.52 | 3.4 | 39 | −18 |
| 8 | 84 | 8 | 90.52 | 3.2 | 39 | −20 |
| 10 | 82 | 8 | 90.52 | 3.1 | 39 | −21 |
| 12 | 78 | 10 | 112.9 | 2.7 | 38 | −26 |
| 12 | 76 | 12 | 135.28 | 2.4 | 38 | −28 |
| 12 | 80 | 8 | 90.52 | 2.8 | 37 | −24 |
| 8 | 86 | 6 | 68.14 | 3.2 | 37 | −18 |
| 10 | 84 | 6 | 68.14 | 3.1 | 37 | −20 |
| 6 | 88 | 6 | 68.14 | 3.3 | 37 | −16 |
| 14 | 76 | 10 | 112.9 | 2.4 | 37 | −27 |
| 4 | 90 | 6 | 68.14 | 3.4 | 37 | −15 |
| 12 | 82 | 6 | 68.14 | 3.0 | 37 | −21 |
| 2 | 92 | 6 | 68.14 | 3.4 | 36 | −13 |
| 14 | 78 | 8 | 90.52 | 2.6 | 36 | −26 |
| 14 | 80 | 6 | 68.14 | 2.8 | 36 | −24 |
| 10 | 86 | 4 | 45.76 | 3.0 | 35 | −18 |
| 12 | 84 | 4 | 45.76 | 3.0 | 35 | −20 |
| 8 | 88 | 4 | 45.76 | 3.1 | 35 | −16 |
| 16 | 76 | 8 | 90.52 | 2.4 | 34 | −28 |
| 16 | 78 | 6 | 68.14 | 2.6 | 34 | −26 |
| 14 | 82 | 4 | 45.76 | 2.8 | 34 | −21 |
| 6 | 90 | 4 | 45.76 | 3.1 | 34 | −14 |
| 4 | 92 | 4 | 45.76 | 3.0 | 34 | −13 |
| 16 | 80 | 4 | 45.76 | 2.7 | 34 | −23 |
| 2 | 94 | 4 | 45.76 | 2.9 | 33 | −11 |
| 18 | 76 | 6 | 68.14 | 2.3 | 33 | −28 |
| 18 | 78 | 4 | 45.76 | 2.5 | 33 | −25 |
| 14 | 84 | 2 | 23.38 | 2.7 | 33 | −19 |
| 12 | 86 | 2 | 23.38 | 2.8 | 33 | −18 |
| 10 | 88 | 2 | 23.38 | 2.8 | 32 | −16 |
| 16 | 82 | 2 | 23.38 | 2.7 | 32 | −21 |
| 8 | 90 | 2 | 23.38 | 2.7 | 32 | −14 |
| 18 | 80 | 2 | 23.38 | 2.5 | 32 | −23 |
| 20 | 78 | 2 | 23.38 | 2.4 | 31 | −25 |
| 6 | 92 | 2 | 23.38 | 2.6 | 31 | −12 |
| 20 | 76 | 4 | 45.76 | 2.3 | 31 | −28 |
| 4 | 94 | 2 | 23.38 | 2.4 | 30 | −11 |
| 22 | 74 | 4 | 45.76 | 2.0 | 29 | −30 |
| 22 | 76 | 2 | 23.38 | 2.2 | 29 | −27 |
| 2 | 96 | 2 | 23.38 | 2.1 | 29 | −9 |
| 24 | 74 | 2 | 23.38 | 2.0 | 28 | −29 |

R-1234zeZ, R-1234zeE and R-134 at 94 wt. %, 4 wt. % and 2 wt. %, respectively, represents the maximum COP for heating maintaining a low glide of <3 K. At this maximum efficiency, the volumetric heating capacity is 39% greater than incumbent R-1223zdE, while the efficiency is-16% variation of the efficiency of R-1233zdE.

### OTHER EMBODIMENTS

Embodiment 1: A composition comprising Z-1,3,3,3-tetrafluoropropene and at least one additional compound selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

Embodiment 2: The composition of Embodiment 1, wherein the total amount of the additional compounds is greater than 0 and less than 1%, preferably greater than 0 and less than 0.4%, and more preferably greater than 0 and less than 0.1%, based on the total composition.

Embodiment 3: The composition of any of Embodiments 1-2, the composition further comprising at least one compound selected from HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224 yd(E), CFO-1112(E), CFO-1112 (Z), HFC-245fa, HFC-236fa, HFC-227ea, trans-1,2-dichloroethylene, HFO-1132(E), HFO-1132 (Z), HFC-152a, HFC-134a, HFC-134, HFC-32, HFC-125, carbon dioxide, isobutene, propane, butane, isobutane, pentane and isopentane.

Embodiment 4: A composition comprising Z-1,3,3,3-tetrafluoropropene and at least one compound selected from HFO-1336mzz(E), HFO-1336mzz(Z), HFC-245fa, HFC-227ea, trans-1,2-dichloroethylene, HFC-134, and isobutane.

Embodiment 5: A composition comprising one of:
Z-1,3,3,3-tetrafluoropropene, HFO-1336mzz(E) and HFO-1336mzz(Z);
Z-1,3,3,3-tetrafluoropropene and HFO-1336mzz(E);
Z-1,3,3,3-tetrafluoropropene and HFO-1336mzz(Z);
Z-1,3,3,3-tetrafluoropropene and at least one compound selected from the group consisting of HFO-1336mzz(Z), HFO-1336mzz(E), HCFO-1233zd(E), HCFO-1233zd(Z), HFO-1234ze(E), HFC-236fa, HFC-134, HFC-152a, HFC-245fa, HFO-1132(E), and HFO-1132 (Z);
Z-1,3,3,3-tetrafluoropropene, HFO-1336mzz(E) and HFC-245fa;
Z-1,3,3,3-tetrafluoropropene, HFO-1336mzz(E) and isobutane;
Z-1,3,3,3-tetrafluoropropene, HFO-1336mzz(E) and HFC-227ea;
Z-1,3,3,3-tetrafluoropropene, HFO-1336mzz(E), HFO-1336mzz(Z) and HFC-134;
Z-1,3,3,3-tetrafluoropropene, HFO-1336mzz(Z) and HCO-1130(E); and
Z-1,3,3,3-tetrafluoropropene, HFO-1336mzz(Z) and HCFO-1224 yd(Z).

In one aspect of Embodiment 5, all of the compositions of Embodiment 5 are free of or substantially free of Group A Fluorinated Substances. In another aspect of Embodiment 5, degradation products of some of the compositions of Embodiment 5 are free of or substantially free of Group A Fluorinated Substances.

Embodiment 6: The composition of any of Embodiments 1-5, wherein the composition comprises a refrigerant.

Embodiment 7: The composition of Embodiment 5, wherein the composition is one of (i), (ii) or (iii), and further comprises: one or more additional compounds selected from HCFO-1233xf, HFO-1336ft, HCFC-133a, CO-1140, HCFO-1233zd(E), HFC-245fa, HFO-1327mz, HFC-347mef, HFO-1243zf; one or more additional compounds selected from HFO-1336mzz(E), HFO-1327mz, HFO-1326mxz(Z), HFO-1326mxz(E), HFC-356mff, CHFC-346mdf, and HFC-263fb; and/or one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

Embodiment 8: The composition of Embodiment 5, wherein the composition is (i), and further comprises Z-1,3,3,3-tetrafluoropropene in an amount of 27 wt % to 72 wt %, HFO-1336mzz(E) in an amount of 0.5 wt % to 70.5 wt %, and %, HFO-1336mzz(Z) in an amount of 0.5 wt % to 27.5 wt %, based on the total weight of the composition.

Embodiment 9: The composition of Embodiment 5, wherein the composition is (iii), and further comprises Z-1,3,3,3-tetrafluoropropene in an amount of about 72 wt % to about 99.5 wt %, and HFO-1336mzz(Z) in an amount of about 0.5 wt % to about 28 wt %, based on the total weight of the composition.

Embodiment 10: The composition of Embodiment 5, wherein the composition is (ii), and further comprises Z-1,3,3,3-tetrafluoropropene in an amount of about 0.5 wt % to about 99.5 wt %, and HFO-1336mzz(E) in an amount of about 0.5 wt % to about 99.5 wt %, based on the total weight of the composition.

Embodiment 11: The composition of any of the preceding Embodiments, wherein the composition is azeotropic or azeotropic-like.

Embodiment 12: The composition of any of Embodiments 1-11, wherein the composition has a GWP of 300 or less, preferably 150 or less.

Embodiment 13: The composition of any of Embodiments 1-12, wherein the composition has a flammability classification of 1 or 2 L as determined by ASHRAE Standard 34 and ASTM E681-09.

Embodiment 14: The composition according to any of Embodiments 1-13, the composition further comprising at least one lubricant.

Embodiment 15: The composition according to any of Embodiments 1-14, the composition further comprising at least one lubricant selected from the group consisting of polyalkylene glycols, polyol esters, polyvinyl ethers, poly-alpha-olefins, and combinations thereof.

Embodiment 16: The composition according to any of Embodiments 1-15, the composition comprising HFO-1234yf and further comprising at least one olefin polymerization inhibitor.

Embodiment 17: The composition according to any of Embodiments 1-16, the composition comprising HFO-1234yf and further comprising at least one olefin polymerization inhibitor selected from the group consisting of d-limonene, l-limonene, β-pinene, a-pinene, a-terpinene, β-terpinene, y-terpinene, and δ-terpinene, ethane, propane, cyclopropane, propylene, butane, butene, isobutane, isobutene, 2-methylbutane, meta-, ortho- or para-xylene, alpha, methyl styrene; alpha, 2-dimethyl styrene; alpha, 3-dimethyl styrene, alpha, 4-dimethyl styrene, and combinations thereof.

Embodiment 18: The composition according to any of Embodiments 1-17, the composition further comprising at least one acid scavenger.

Embodiment 19: The composition according to any of Embodiments 1-18, the composition further comprising at least one acid scavenger selected from the group consisting of epoxides, amines and hindered amines.

Embodiment 20: A method of producing Z-1,3,3,3-tetrafluoropropene, the method comprising contacting HFC-245fa in the gas phase with a catalyst, in the presence of an oxygen containing gas, to form a reaction mixture comprising Z-1,3,3,3-tetrafluoropropene, E-1,3,3,3-tetrafluoropropene, and optionally additional constituents.

Embodiment 21: The method of Embodiment 20, wherein the catalyst comprises chromium.

Embodiment 22: A method of producing Z-1,3,3,3-tetrafluoropropene, the method comprising reacting HCC-240fa with a fluorinating agent, in the presence of a fluorinated catalyst, to form a reaction mixture comprising Z-1,3,3,3-tetrafluoropropene, E-1,3,3,3-tetrafluoropropene, and optionally additional constituents.

Embodiment 23: A method of producing Z-1,3,3,3-tetrafluoropropene, the method comprising reacting HCFO-1233zd with a fluorinating agent, in the presence of a fluorinated catalyst, to form a reaction mixture comprising Z-1,3,3,3-tetrafluoropropene, E-1,3,3,3-tetrafluoropropene, and optionally additional constituents.

Embodiment 24: The method of any of Embodiments 22 and 23, wherein the fluorinating agent is hydrogen fluoride.

Embodiment 25: The method of Embodiment 24, wherein the hydrogen fluoride is anhydrous or substantially anhydrous.

Embodiment 26: The method of any of Embodiments 20, 22 and 23, the method further comprising: separating the Z-1,3,3,3-tetrafluoropropene from other constituents of the reaction mixture; and recovering the Z-1,3,3,3-tetrafluoropropene.

Embodiment 27: The method of any of Embodiments 22 and 23, wherein the fluorinated catalyst is fluorinated Cr₂O₃.

Embodiment 28: The method of any of Embodiments 20-27, the method further comprising recovering the E-1,3,3,3-tetrafluoropropene and converting the recovered E-1,3,3,3-tetrafluoropropene to the Z-isomer.

Embodiment 29: The method of Embodiment 28, wherein the converting comprises contacting the E-1,3,3,3-tetrafluoropropene in the gas phase with at least one fluorinated catalyst, optionally in the presence of an oxygen containing gas, to form Z-1,3,3,3-tetrafluoropropene.

Embodiment 30: The method of any of Embodiments 28-29, wherein 2% to 50% of the E-1,3,3,3-tetrafluoropropene is converted to the Z-isomer.

Embodiment 31: A system for cooling or heating comprising an evaporator, compressor, condenser, and expansion device, said system containing the composition of any of Embodiments 1 to 19, particularly the composition of any of Embodiments 1, 3, 4, 5, 7, 8, 9 and 10.

Embodiment 32: A method for producing heating in a high temperature heat pump, the method comprising condensing the composition of any of Embodiments 1 to 19, particularly the composition of any of Embodiments 1, 3, 4, 5, 7, 8, 9 and 10, in a condenser, wherein the high temperature heat pump uses condenser operating temperatures greater than about 55° C., preferably from about 55° C. to about 150° C.

Embodiment 33: A high temperature heat pump comprising a condenser and the composition of any of Embodiments 1 to 19, particularly the composition of any of Embodiments 1, 3, 4, 5, 7, 8, 9 and 10, wherein an operating temperature of the condenser is greater than about 55° C., greater than 65° C., greater than 75° C., greater than 85° C., greater than 95° C., greater than 100° C. to about 150° C., greater than about 150° C., greater than about 160° C., or greater than about 170° C.

Embodiment 34: A refrigerant charging kit comprising: the composition of any of Embodiments 1 to 19, particularly the composition of any of Embodiments 1, 3, 4, 5, 7, 8, 9 and 10, in a sealed canister, the composition optionally further comprising carbon dioxide, and a tube for connecting a discharge end of the sealed canister to a valve of a refrigerant circuit.

Embodiment 35: A method for replacing a first refrigerant composition with a second refrigerant composition in a cooling or heating system, the method comprising removing the first refrigerant composition from the cooling or heating system and charging second refrigerant composition to the cooling or heating system, wherein the first refrigerant is selected from the group consisting of HCFO-1233zdE, HFO-1336mzzE and HFO-1336mzzZ, and wherein the second refrigerant composition is the composition of any of Embodiments 1 to 19, particularly the composition of any of Embodiments 1, 3, 4, 5, 7, 8, 9 and 10.

Embodiment 36: A method for reclaiming the composition of any one of Embodiments 1 to 19, particularly the composition of any of Embodiments 1, 3, 4, 5, 7, 8, 9 and 10, the method comprising: a) providing or obtaining the composition of any one of claims 1 to 25 in a used state; b) testing the used composition to determine purity of the composition and if the composition comprises contaminants and/or non-condensable gases; c) checking and comparing the purity of the used refrigerant composition relative to AHRI 700 standards; and d) if the used refrigerant composition of does not meet AHRI 700 standards, treating and purifying the used refrigerant composition and providing at least one first treated product; and optionally repeating the procedure on the first treated product if needed to meet AHRI 700 standards; and optionally adding additional refrigerant components to the first treated product to form a first target refrigerant or refrigerant blend if the first treated product meets or exceeds AHRI 700 standards, or further purifying the first treated product that does not meet AHRI 700 standards to produce a second treated product and repeating the procedure as needed to obtain a second treated product which meets or exceeds AHRI 700 standards.

Embodiment 37: A high temperature heat pump comprising a condenser, a flooded evaporator, and the composition of any of Embodiments 1 to 19, particularly the composition of any of Embodiments 1, 3, 4, 5, 7, 8, 9 and 10, wherein the composition has a critical temperature which is at least 5° C. greater than an operating temperature of the condenser.

Embodiment 38: The high temperature heat pump of Embodiment 37, wherein the composition has a critical temperature of at least about 60° C., preferably at least about 135° C., and most preferably at least about 155° C.

Embodiment 39: A method of raising an operating temperature of a condenser of a high temperature heat pump apparatus, the method comprising charging the high temperature heat pump with a working fluid comprising the composition of any of Embodiments 1 to 19 particularly the composition of any of Embodiments 1, 3, 4, 5, 7, 8, 9 and 10.

Embodiment 40: The method of Embodiment 39, wherein the condenser operating temperature is raised to a temperature greater than about 150° C.

Embodiment 41: A high temperature heat pump circuit containing the composition of any of Embodiments 1 to 19 particularly the composition of any of Embodiments 1, 3, 4, 5, 7, 8, 9 and 10.

Embodiment 42: A high temperature heat pump containing the composition of any of Embodiments 1 to 19 particularly the composition of any of Embodiments 1, 3, 4, 5, 7, 8, 9 and 10.

Embodiment 43: A flooded evaporator chiller for cooling or heating, the chiller containing the composition of any of Embodiments 1 to 19 particularly the composition of any of Embodiments 1, 3, 4, 5, 7, 8, 9 and 10.

Embodiment 44: A method for producing cooling in a flooded evaporator chiller, the method comprising evaporating the composition of any of Embodiments 1 to 19 particularly the composition of any of Embodiments 1, 3, 4, 5, 7, 8, 9 and 10, in a flooded evaporator.

Embodiment 45: The method of Embodiment 44, wherein the cooling or heating system is one of a flooded evaporator heat pump or a flooded evaporator chiller.

Embodiment 46: A composition comprising Z-1,3,3,3-tetrafluoropropene and one or more additional compounds selected from HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb, the composition being are free of or substantially free of Group A Fluorinated Substances.

Embodiment 47: The composition of Embodiment 45, wherein degradation products of the composition are free of or substantially free of Group A Fluorinated Substances.

Embodiment 48: A composition comprising Z-1,3,3,3-tetrafluoropropene and one or more compounds selected from HFO-1336mzz(Z), HFO-1336mzz(E), HCFO-1233zd(E), HCFO-1233zd(Z), HFO-1234ze(E), HFC-236fa, HFC-134, HFC-152a, HFC-245fa, HFO-1132(E), and HFO-1132 (Z); wherein the composition is free of or substantially free of Group A Fluorinated Substances.

Embodiment 49: The composition of Embodiment 48, wherein degradation products of the composition are free of or substantially free of Group A Fluorinated Substances, such as TFA.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A composition comprising Z-1,3,3,3-tetrafluoropropene (HFO-1234ze(Z)) and at least one additional compound selected from the group consisting of HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb.

2. The composition according to claim 1, wherein the total amount of the additional compounds is greater than 0 and less than 1%, preferably greater than 0 and less than 0.4%, and more preferably greater than 0 and less than 0.1%, based on the total composition.

3. The composition according to claim 1, the composition further comprising at least one compound selected from the group consisting of HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224yd(E), CFO-1112(E), CFO-1112(Z), HFC-245fa, HFC-236fa, HFC-227ea, trans-1,2-dichloroethylene, HFO-1132(E), HFO-1132(Z), HFC-152a, HFC-134a, HFC-134, HFC-32, HFC-125, carbon dioxide, isobutene, propane, butane, isobutane, pentane and isopentane.

4. A composition comprising Z-1,3,3,3-tetrafluoropropene (HFO-1234ze(Z)) and at least one compound selected from the group consisting of HFO-1234yf, HFO-1234ze(E), HFO-1225ye, HFO-1336mzz(E), HFO-1336mzz(Z), HFO-1336yf, HFO-1336ze(E), HFO-1336ze(Z), HCFO-1233zd(E), HCFO-1233zd(Z), HCFO-1224 yd(Z), HCFO-1224yd(E), CFO-1112(E), CFO-1112(Z), HFC-245fa, HFC-236fa, HFC-227ea, trans-1,2-dichloroethylene, HFO-1132(E), HFO-1132(Z), HFC-152a, HFC-134a, HFC-134, HFC-32, HFC-125, carbon dioxide, isobutene, propane, butane, isobutane, pentane and isopentane.

5. The composition according to claim 4, wherein the composition comprises (i) HFO-1234ze(Z), HFO-1336mzz(E) and HFO-1336mzz(Z); or (ii) HFO-1234ze(Z) and HFO-1336mzz(E); or (iii) HFO-1234ze(Z) and HFO-1336mzz(Z).

6. The composition according to claim 5, wherein:
composition (i) comprises (a) one or more additional compounds selected from the group consisting of HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; (b) one or more additional compounds selected from the group consisting of HCFO-1233xf, HFO-1336ft, HCFC-133a, CO-1140, HCFO-1233zd(E), HFC-245fa, HFO-1327mz, HFC-347mef, HFO-1243zf; and (c) one or more additional compounds selected from the group consisting of HFO-1336mzz(E), HFO-1327mz, HFO-1326mxz(Z), HFO-1326mxz(E), HFC-356mff, CHFC-346mdf, and HFC-263fb; or
composition (ii) comprises (a) one or more additional compounds selected from the group consisting of HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (b) one or more additional compounds selected from the group consisting of HCFO-1233xf, HFO-1336ft, HCFC-133a, CO-1140, HCFO-1233zd(E), HFC-245fa, HFO-1327mz, HFC-347mef, HFO-1243zf; or
composition (iii) comprises (a) one or more additional compounds selected from the group consisting of HFO-1234ze(E), HFC-263fb, HFO-1234zc, HFC-245fa, HCFO-1233zd(E), HCFO-1233zd(Z), HCFO-1233xf, HCFC-124, HCC-40, CFC-114, HCFO-1131(E), CFC-114a, HCFC-124a, HFC-227ca, HFO-1234yf, HFC-152a, HFO-1243zf, HCC-30, HFC-134a, HFC-236fa, HFO-1327 isomer, HFO-1336mzz(E), CFO-1112a, HFC-227ea and HFC-245cb; and (b) one or more additional compounds selected from the group consisting of HFO-1336mzz(E), HFO-1327mz, HFO-1326mxz(Z), HFO-1326mxz(E), HFC-356mff, CHFC-346mdf, and HFC-263fb.

7. The composition according to claim 5, wherein:
the composition (i) comprises HFO-1234ze(Z) in an amount of 27 wt % to 72 wt %, HFO-1336mzz(E) in an amount of 0.5 wt % to 70.5 wt %, and HFO-1336mzz(Z) in an amount of 0.5 wt % to 27.5 wt %, based on the total weight of the composition; or
the composition (ii) comprises HFO-1234ze(Z) in an amount of about 0.5 wt % to about 99.5 wt %, and HFO-1336mzz(E) in an amount of about 0.5 wt % to about 99.5 wt %, based on the total weight of the composition; or
the composition (iii) comprises HFO-1234ze(Z) in an amount of about 72 wt % to about 99.5 wt %, and HFO-1336mzz(Z) in an amount of about 0.5 wt % to about 28 wt %, based on the total weight of the composition.

8. The composition according to claim 4, wherein the composition is azeotropic or azeotropic-like.

9. The composition according to any one of the preceding claims, the composition further comprising at least one lubricant selected from the group consisting of polyalkylene glycols, polyol esters, polyvinyl ethers, poly-alpha-olefins, and combinations thereof.

10. A method of producing Z-1,3,3,3-tetrafluoropropene (HFO-1234ze(Z)), the method comprising contacting an organic compound in the gas phase with a catalyst, in the presence of one of an oxygen containing gas and a fluorinating agent, to form a reaction mixture comprising HFO-1234ze(Z).

11. The method according to claim 10, wherein the reaction mixture further comprises E-1,3,3,3-tetrafluoropropene (HFO-1234ze(E) and optionally additional constituents.

12. The method according to claim 11, the method further comprising:
separating the HFO-1234ze(Z) from other constituents of the reaction mixture; and
recovering the HFO-1234ze(Z).

13. The method according to claim 12, the method further comprising recovering the E HFO-1234ze(E) and converting the recovered HFO-1234ze(E) to the Z-isomer.

14. The method according to claim 10, wherein the organic compound is 1,1,1,3,3-pentafluoropropane (HFC-245fa), and wherein the method comprises dehydrofluorination of the HFC-245a by contacting the HFC-245fa in the gas phase with the catalyst in the presence of the oxygen containing gas.

15. The method according to claim 14, wherein the dehydrofluorination reaction is carried out at a temperature of between about 200° C. to about 400° C.

16. The method according to claim 14, wherein the catalyst comprises chromium.

17. The method according to claim 10, wherein the organic compound is 1,1,1,3,3-pentachloropropane (HCC-240fa) or 1-chloro-3,3,3-trifluoropropene (HCFO-1233zd), and wherein the method comprises fluorination of the HCC-240fa or HCFO-1233zd by contacting the HCC-240fa or HCFO-1233zd in the gas phase with the catalyst and a fluorinating agent.

18. The method according to claim 17, wherein the fluorinating agent is hydrogen fluoride.

19. The method according to claim 17, wherein the fluorinated catalyst is fluorinated Cr₂O₃.

20. The method according to claim 17, wherein the fluorination reaction is conducted at a temperature ranging from about 80° C. to about 400° C.
